SEQUENCE LISTING (152 sequences)

SEQ ID NO: 1 - RNA, 2216 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaugauaauaggcuggagccucgguggccuagcuucuugccccuugggccuccccccagccccuccuccccuuccugcacccguacccccguggucuuugaauaaagucugagugggcggc

SEQ ID NO: 2 - RNA, 57 nt, Artificial Sequence
  Synthetic
gggaaauaagagagaaaagaagaguaagaagaaauauaagaccccggcgccgccacc

SEQ ID NO: 3 - RNA, 2040 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauuca

SEQ ID NO: 4 - RNA, 119 nt, Artificial Sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 5 - protein, 680 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNS

SEQ ID NO: 6 - RNA, 2729 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaggaggaggcagcggcggcgauaucaucaagcuucugaacgagcaaguuaacaaggaaaugcagagcaguaaucucuacaugagcaugagcagcuggugcuacacccacucccuggacggagcaggccucuuccuguucgaccacgcagccgaggaguacgagcacgcuaagaaguugaucauuuucuugaacgagaacaacgugcccgugcagcuaacgucaaucagcgcaccugagcacaaguucgagggccugacccagaucuuccagaaggccuacgaacacgaacagcacaucuccgagagcaucaacaauauuguggaucacgcuaucaaguccaaggaccacgcuaccuucaacuuccugcagugguacguggccgagcaacaugaggaggaggugcuguucaaggacauccuggacaagaucgagcugaucgguaaugagaaucacggccuguaccuggccgaccaguacgugaagggcaucgccaagagccggaagucaggcucaugauaauaggcuggagccucgguggccuagcuucuugccccuugggccuccccccagccccuccuccccuuccugcacccguacccccguggucuuugaauaaagucugagugggcggc

SEQ ID NO: 7 - RNA, 2553 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaggaggaggcagcggcggcgauaucaucaagcuucugaacgagcaaguuaacaaggaaaugcagagcaguaaucucuacaugagcaugagcagcuggugcuacacccacucccuggacggagcaggccucuuccuguucgaccacgcagccgaggaguacgagcacgcuaagaaguugaucauuuucuugaacgagaacaacgugcccgugcagcuaacgucaaucagcgcaccugagcacaaguucgagggccugacccagaucuuccagaaggccuacgaacacgaacagcacaucuccgagagcaucaacaauauuguggaucacgcuaucaaguccaaggaccacgcuaccuucaacuuccugcagugguacguggccgagcaacaugaggaggaggugcuguucaaggacauccuggacaagaucgagcugaucgguaaugagaaucacggccuguaccuggccgaccaguacgugaagggcaucgccaagagccggaagucaggcuca

SEQ ID NO: 8 - protein, 851 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSGGGSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 9 - RNA, 2762 nt, Artificial Sequence
  Synthetic
gggaaauaagagagaaaagaagaguaagaagaaauauaagaccccggcgccgccaccaugggcauccugcccagcccuggcaugcccgcucugcugagccuggugagccugcugagcgugcugcugaugggcugcguggcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggggcggaaguggaggcucugguggcagcggaggaucuggcggcggcaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaugauaauaggcuggagccucgguggccuagcuucuugccccuugggccuccccccagccccuccuccccuuccugcacccguacccccguggucuuugaauaaagucugagugggcggc

SEQ ID NO: 10 - RNA, 2586 nt, Artificial Sequence
  Synthetic
augggcauccugcccagcccuggcaugcccgcucugcugagccuggugagccugcugagcgugcugcugaugggcugcguggcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggggcggaaguggaggcucugguggcagcggaggaucuggcggcggcaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauuca

SEQ ID NO: 11 - protein, 862 aa, Artificial Sequence
  Synthetic
MGILPSPGMPALLSLVSLLSVLLMGCVAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNS

SEQ ID NO: 12 - RNA, 2714 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaggaggaggcuccggaggcgguagcgcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggugauaauaggcuggagccucgguggccuagcuucuugccccuugggccuccccccagccccuccuccccuuccugcacccguacccccguggucuuugaauaaagucugagugggcggc

SEQ ID NO: 13 - RNA, 2538 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaggaggaggcuccggaggcgguagcgcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcgg

SEQ ID NO: 14 - protein, 846 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSGGGSGGGSAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 15 - RNA, 2300 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 16 - RNA, 2124 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaucuggcggaggcagcauccuggccaucuacagcaccguggccagcagccuggugcugcuggugagccugggcgccaucagcuuc

SEQ ID NO: 17 - protein, 707 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSGGGSILAIYSTVASSLVLLVSLGAISF

SEQ ID NO: 18 - RNA, 1853 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 19 - RNA, 1677 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccucuggcggaggcagcauccuggccaucuacagcaccguggccagcagccuggugcugcuggugagccugggcgccaucagcuuc

SEQ ID NO: 20 - protein, 559 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTSGGGSILAIYSTVASSLVLLVSLGAISF

SEQ ID NO: 21 - RNA, 2042 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 22 - RNA, 1866 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuuc

SEQ ID NO: 23 - protein, 622 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGSGGGSILAIYSTVASSLVLLVSLGAISF

SEQ ID NO: 24 - RNA, 2066 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccgugggcagcggcggcggcagcggcggaggcagcggaggaggcagcggcggaggcaguggaggccagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 25 - RNA, 1890 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccgugggcagcggcggcggcagcggcggaggcagcggaggaggcagcggcggaggcaguggaggccagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuuc

SEQ ID NO: 26 - protein, 630 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVGSGGGSGGGSGGGSGGGSGGQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGSGGGSILAIYSTVASSLVLLVSLGAISF

SEQ ID NO: 27 - RNA, 2066 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccgugggcagcggcggcggcagcggcggaggcagcggaggaggcagcggcggaggcaguggaggccagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuuugccaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 28 - RNA, 1890 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccgugggcagcggcggcggcagcggcggaggcagcggaggaggcagcggcggaggcaguggaggccagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuuugccaguucggccgggacaucgccgacaccaccgacgcugugcgggauccccagacccuggagauccuggacaucaccccuugcagcuucggcggcucuggcggaggcagcauccuggccaucuacagcaccguggccagcagccuggugcugcuggugagccugggcgccaucagcuuc

SEQ ID NO: 29 - protein, 630 aa, Artificial Sequence
  Synthetic
MFVFLVLLPLVSSQCVNLTTRTQLPPAYTNSFTRGVYYPDKVCRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVGSGGGSGGGSGGGSGGGSGGQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFCQFGRDIADTTDAVRDPQTLEILDITPCSFGGSGGGSILAIYSTVASSLVLLVSLGAISF

SEQ ID NO: 30 - RNA, 3182 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuucggcggcagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccaacgccaccgugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 31 - RNA, 3006 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuucggcggcagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccaacgccaccgugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 32 - protein, 1002 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFGGSGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPNATVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 33 - RNA, 2378 nt, Artificial Sequence
  Synthetic
gggaaauaagagagaaaagaagaguaagaagaaauauaagaccccggcgccgccaccauguucguguuccuggugcugcugccccuggugagcagccagggcaccaucaccgacgccguggacugcgcccuggacccucugagcgagaccaagugcacccugaagagcuucaccguggagaagggcaucuaccagaccagcaacuucggcggcagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugaacuacaccagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccaacgccaccgugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 34 - RNA, 2202 nt, Artificial Sequence
  Synthetic
auguucguguuccuggugcugcugccccuggugagcagccagggcaccaucaccgacgccguggacugcgcccuggacccucugagcgagaccaagugcacccugaagagcuucaccguggagaagggcaucuaccagaccagcaacuucggcggcagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugaacuacaccagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccaacgccaccgugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 35 - protein, 734 aa, Artificial Sequence
  Synthetic
MFVFLVLLPLVSSQGTITDAVDCALDPLSETKCTLKSFTVEKGIYQTSNFGGSGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLNYTSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPNATVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 36 - RNA, 4241 nt, Artificial Sequence
  Synthetic
gggaaauaagagagaaaagaagaguaagaagaaauauaagaccccggcgccgccaccauguuccugaucaucuucauccugcccaccacccuggccgugaucggcgacuucaacugcaccaacagcuucaucaacgacuacaacaagaccaucccucggaucagcgaagacgugguggacgugucccugggccugggcaccuacuacgugcugaaccggguguaccugaacaccacacugcuguucaccggcuacuuccccaagagcggcgccaacuuccgggaccuggcccugaagggcagcaucuaccugagcaccuugugguacaagccucccuuccugagcgacuucaauaacggcaucuucucuaaggugaagaacaccaagcuguacguaaacaacacccuguacagcgaguucagcaccaucgugaucggcagcguguucgucaacaccagcuacaccaucguggugcagccccacaacggcauccuggagaucaccgccugccaguacaccaugugcgaguacccucacaccgugugcaagagcaagggcuccauccggaacgagagcuggcacaucgacagcagcgagccgcugugccuguucaagaagaacuucaccuacaacgugagcgccgacuggcuguacuuccacuucuaccaggagcggggcguguucuacgccuacuacgccgacgugggcaugccaaccaccuuccuguucagccuguaccugggcaccauccugagccacuacuacgugaugccccugaccugcaacgccaucagcucaaacaccgacaacgagacccuggaguacugggugacuccacugagccggcggcaguaccugcugaacuucgacgagcacggcgugaucaccaacgccguggacugcgcccuggacccucugagcgagaccaagugcacccugaagagcuucaccguggagaagggcaucuaccagaccagcggcuucaccgugaagcccguagccaccguguaccggcggauccccaaccugcccgacugcgacaucgacaacuggcugaacaacgucagcgugcccagcccacugaacugggagcggcggaucuucagcaacugcaacuucaaucugagcacccugcugcggcuggugcacguggacagcuucagcugcaacaaccuggacaagagcaagaucuucgguagcugcuucaacagcaucaccguggacaaguucgccaucccuaaccggcggcgggacgaucugcagcugggcagcagcggcuuccugcagagcagcaacuacaagaucgacaucagcagcucaagcugccagcuguacuacagccugccccuggugaacgugaccaucaacaacuucaaccccagcagcuggaaccggcgguacggcuucggcagcuucaaccugagcagcuacgacgugguguacagcgaccacugcuucagcgugaacagcgacuucugccccugugccgacccuagcguggugaacagcugcgccaagagcaagccucccagcgccauuugccccgccggcaccaaguaccggcacugcgaccuggacaccacccuguacgugaagaacuggugccggugcagcugccugcccgaccccaucagcaccuacagccccaacaccuguccccagaagaagguggugguggguaucggcgagcacugucccggccugggcaucaacgaggagaagugcggcacccagcugaaccacagcagcugcuucuguagccccgacgccuuccugggcuggagcuucgacagcugcaucagcaacaaccggugcaacaucuuuagcaacuucaucuucaacggaaucaacagcggcaccaccugcagcaacgaccugcuguauagcaacaccgagaucagcaccggcgugugcgugaacuacgaccuguacggcaucaccggccagggcaucuucaaggaggugagcgccgccuacuacaacaacuggcagaaccugcuguacgacagcaacggcaacaucaucggcuucaaggacuuucugaccaacaagaccuacaccauccugcccugcuacagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 37 - RNA, 4065 nt, Artificial Sequence
  Synthetic
auguuccugaucaucuucauccugcccaccacccuggccgugaucggcgacuucaacugcaccaacagcuucaucaacgacuacaacaagaccaucccucggaucagcgaagacgugguggacgugucccugggccugggcaccuacuacgugcugaaccggguguaccugaacaccacacugcuguucaccggcuacuuccccaagagcggcgccaacuuccgggaccuggcccugaagggcagcaucuaccugagcaccuugugguacaagccucccuuccugagcgacuucaauaacggcaucuucucuaaggugaagaacaccaagcuguacguaaacaacacccuguacagcgaguucagcaccaucgugaucggcagcguguucgucaacaccagcuacaccaucguggugcagccccacaacggcauccuggagaucaccgccugccaguacaccaugugcgaguacccucacaccgugugcaagagcaagggcuccauccggaacgagagcuggcacaucgacagcagcgagccgcugugccuguucaagaagaacuucaccuacaacgugagcgccgacuggcuguacuuccacuucuaccaggagcggggcguguucuacgccuacuacgccgacgugggcaugccaaccaccuuccuguucagccuguaccugggcaccauccugagccacuacuacgugaugccccugaccugcaacgccaucagcucaaacaccgacaacgagacccuggaguacugggugacuccacugagccggcggcaguaccugcugaacuucgacgagcacggcgugaucaccaacgccguggacugcgcccuggacccucugagcgagaccaagugcacccugaagagcuucaccguggagaagggcaucuaccagaccagcggcuucaccgugaagcccguagccaccguguaccggcggauccccaaccugcccgacugcgacaucgacaacuggcugaacaacgucagcgugcccagcccacugaacugggagcggcggaucuucagcaacugcaacuucaaucugagcacccugcugcggcuggugcacguggacagcuucagcugcaacaaccuggacaagagcaagaucuucgguagcugcuucaacagcaucaccguggacaaguucgccaucccuaaccggcggcgggacgaucugcagcugggcagcagcggcuuccugcagagcagcaacuacaagaucgacaucagcagcucaagcugccagcuguacuacagccugccccuggugaacgugaccaucaacaacuucaaccccagcagcuggaaccggcgguacggcuucggcagcuucaaccugagcagcuacgacgugguguacagcgaccacugcuucagcgugaacagcgacuucugccccugugccgacccuagcguggugaacagcugcgccaagagcaagccucccagcgccauuugccccgccggcaccaaguaccggcacugcgaccuggacaccacccuguacgugaagaacuggugccggugcagcugccugcccgaccccaucagcaccuacagccccaacaccuguccccagaagaagguggugguggguaucggcgagcacugucccggccugggcaucaacgaggagaagugcggcacccagcugaaccacagcagcugcuucuguagccccgacgccuuccugggcuggagcuucgacagcugcaucagcaacaaccggugcaacaucuuuagcaacuucaucuucaacggaaucaacagcggcaccaccugcagcaacgaccugcuguauagcaacaccgagaucagcaccggcgugugcgugaacuacgaccuguacggcaucaccggccagggcaucuucaaggaggugagcgccgccuacuacaacaacuggcagaaccugcuguacgacagcaacggcaacaucaucggcuucaaggacuuucugaccaacaagaccuacaccauccugcccugcuacagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 38 - protein, 1355 aa, Artificial Sequence
  Synthetic
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCALDPLSETKCTLKSFTVEKGIYQTSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - RNA, 4247 nt, Artificial Sequence
  Synthetic
gggaaauaagagagaaaagaagaguaagaagaaauauaagaccccggcgccgccaccauguuccugauccugcugaucagccugcccaccgccuucgccgugaucggcgaccugaagugcaccagcgacaacaucaacgacaaggacaccggcccaccacccaucagcaccgacaccguggacgugaccaacggccugggcaccuacuacgugcuggaccggguguaccugaacaccacccuguuccugaacggcuacuaccccaccagcggcagcaccuaccggaauauggcccugaagggcagcgugcugcugagccggcugugguucaagccaccauuccugagcgacuucaucaacggaaucuucgccaaggugaagaacaccaaggugaucaaggaccgggugauguacagcgaguuccccgccaucaccauuggcaguaccuucgugaacaccagcuacagcgugguggugcagccccggaccaucaacagcacccaggacggcgacaacaagcugcagggccugcuggaggugagcgugugccaguacaacaugugcgaguacccucagaccaucugccaccccaaccugggcaaccaccggaaggagcuguggcaccuggacaccggcguggugagcugccuguacaagcggaacuucaccuacgacguaaacgccgacuaccuguacuuccacuucuaccaggagggcggcaccuucuacgccuacuucaccgacacgggcguggugaccaaguuccuguucaacguguaccugggcauggcccugagccacuacuacgugaugccccugaccuguaacagcaagcugacccuggaguacugggugaccccucugaccagccggcaguaccugcuggccuucaaccaggacggcaucaucuucaacgccguggacugcgcccuggacccucugagcgagaccaagugcacccugaagagcuucaccguggagaagggcaucuaccagaccaacggcuacaccgugcagcccaucgccgacguguaccggcggaagcccaaccugcccaacugcaacaucgaggccuggcugaacgacaagagcgugcccucgccccugaacugggagcggaagaccuucagcaacugcaacuucaacaugagcagucugaugagcuucauccaggccgacagcuucaccugcaacaacaucgacgccgccaagaucuacggcaugugcuucagcagcaucaccaucgacaaguuugccauccccaacggccggaagguggaccugcagcugggcaaccugggcuaccugcagagcuucaacuaccggaucgacaccaccgccaccucuugccagcuguacuacaaccugcccgccgccaacgugagcgugagccgguucaaccccagcaccuggaacaagcgguucggcuucauugaggacagcguguucaaaccccggcccgcaggaguacugaccaaccacgacgugguguacgcccagcacugcuucaaggcacccaagaacuucugccccugcaagcugaacggcagcugugugggcucuggccccgguaagaacaacggcauagggacuugcccggcagggaccaacuaccugaccugcgacaaccugugcacacccgaccccaucaccuucaccggcaccuacaaguguccccagaccaagagccuggugggcaucggcgagcacugcagcggccuggccgugaagagcgacuacugcggcggcaacagcugcaccugucggccccaggccuuccugggcuggagcgccgacagcugccugcagggcgacaagugcaacaucuuugccaacuucauccugcacgacgugaacagcggccugaccugcagcaccgaccugcagaaggccaacaccgacaucauccugggcgugugcgugaacuacgacuuguacggcauccugggccagggcaucuucguggaggugaacgccaccuacuacaacagcuggcagaaccugcuguacgacagcaacggcaaccuguacggcuuccgggacuacaucaucaaccggaccuucaugauccggagcugcuacagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 40 - RNA, 4071 nt, Artificial Sequence
  Synthetic
auguuccugauccugcugaucagccugcccaccgccuucgccgugaucggcgaccugaagugcaccagcgacaacaucaacgacaaggacaccggcccaccacccaucagcaccgacaccguggacgugaccaacggccugggcaccuacuacgugcuggaccggguguaccugaacaccacccuguuccugaacggcuacuaccccaccagcggcagcaccuaccggaauauggcccugaagggcagcgugcugcugagccggcugugguucaagccaccauuccugagcgacuucaucaacggaaucuucgccaaggugaagaacaccaaggugaucaaggaccgggugauguacagcgaguuccccgccaucaccauuggcaguaccuucgugaacaccagcuacagcgugguggugcagccccggaccaucaacagcacccaggacggcgacaacaagcugcagggccugcuggaggugagcgugugccaguacaacaugugcgaguacccucagaccaucugccaccccaaccugggcaaccaccggaaggagcuguggcaccuggacaccggcguggugagcugccuguacaagcggaacuucaccuacgacguaaacgccgacuaccuguacuuccacuucuaccaggagggcggcaccuucuacgccuacuucaccgacacgggcguggugaccaaguuccuguucaacguguaccugggcauggcccugagccacuacuacgugaugccccugaccuguaacagcaagcugacccuggaguacugggugaccccucugaccagccggcaguaccugcuggccuucaaccaggacggcaucaucuucaacgccguggacugcgcccuggacccucugagcgagaccaagugcacccugaagagcuucaccguggagaagggcaucuaccagaccaacggcuacaccgugcagcccaucgccgacguguaccggcggaagcccaaccugcccaacugcaacaucgaggccuggcugaacgacaagagcgugcccucgccccugaacugggagcggaagaccuucagcaacugcaacuucaacaugagcagucugaugagcuucauccaggccgacagcuucaccugcaacaacaucgacgccgccaagaucuacggcaugugcuucagcagcaucaccaucgacaaguuugccauccccaacggccggaagguggaccugcagcugggcaaccugggcuaccugcagagcuucaacuaccggaucgacaccaccgccaccucuugccagcuguacuacaaccugcccgccgccaacgugagcgugagccgguucaaccccagcaccuggaacaagcgguucggcuucauugaggacagcguguucaaaccccggcccgcaggaguacugaccaaccacgacgugguguacgcccagcacugcuucaaggcacccaagaacuucugccccugcaagcugaacggcagcugugugggcucuggccccgguaagaacaacggcauagggacuugcccggcagggaccaacuaccugaccugcgacaaccugugcacacccgaccccaucaccuucaccggcaccuacaaguguccccagaccaagagccuggugggcaucggcgagcacugcagcggccuggccgugaagagcgacuacugcggcggcaacagcugcaccugucggccccaggccuuccugggcuggagcgccgacagcugccugcagggcgacaagugcaacaucuuugccaacuucauccugcacgacgugaacagcggccugaccugcagcaccgaccugcagaaggccaacaccgacaucauccugggcgugugcgugaacuacgacuuguacggcauccugggccagggcaucuucguggaggugaacgccaccuacuacaacagcuggcagaaccugcuguacgacagcaacggcaaccuguacggcuuccgggacuacaucaucaaccggaccuucaugauccggagcugcuacagcggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 41 - protein, 1357 aa, Artificial Sequence
  Synthetic
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCALDPLSETKCTLKSFTVEKGIYQTNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYIINRTFMIRSCYSGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 42 - RNA, 1157 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcggcagcgccaucggcggcuacauccccgaggccccuagagacggccaggccuacgugcggaaggacggcgagugggugcugcugagcaccuuccugggcugauaauaggcuggagccucgguggccuagcuucuugccccuugggccuccccccagccccuccuccccuuccugcacccguacccccguggucuuugaauaaagucugagugggcggc

SEQ ID NO: 43 - RNA, 981 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcggcagcgccaucggcggcuacauccccgaggccccuagagacggccaggccuacgugcggaaggacggcgagugggugcugcugagcaccuuccugggc

SEQ ID NO: 44 - protein, 327 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGGSAIGGYIPEAPRDGQAYVRKDGEWVLLSTFLG

SEQ ID NO: 45 - RNA, 1130 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 46 - RNA, 954 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuuc

SEQ ID NO: 47 - protein, 318 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGSILAIYSTVASSLVLLVSLGAISF

SEQ ID NO: 48 - RNA, 1238 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaagugauaauaggcuggagccucgguggccuagcuucuugccccuugggccuccccccagccccuccuccccuuccugcacccguacccccguggucuuugaauaaagucugagugggcggc

SEQ ID NO: 49 - RNA, 1062 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaag

SEQ ID NO: 50 - protein, 354 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGSILAIYSTVASSLVLLVSLGAISFGGGSAIGGYIPEAPRDGQAYVRKDGEWVLLSTFLGK

SEQ ID NO: 51 - RNA, 1130 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 52 - RNA, 954 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacucuggcggaggcagcauccuggccaucuacagcaccguggccagcagccuggugcugcuggugagccugggcgccaucagcuuc

SEQ ID NO: 53 - protein, 318 aa, Artificial Sequence
  Synthetic
MFVFLVLLPLVSSQCVNLTTRTQLPPAYTNSFTRGVYYPDKVCRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDSGGGSILAIYSTVASSLVLLVSLGAISF

SEQ ID NO: 54 - RNA, 1181 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 55 - RNA, 1005 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaaggugugccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccucuggcggaggcagcauccuggccaucuacagcaccguggccagcagccuggugcugcuggugagccugggcgccaucagcuuc

SEQ ID NO: 56 - protein, 335 aa, Artificial Sequence
  Synthetic
MFVFLVLLPLVSSQCVNLTTRTQLPPAYTNSFTRGVYYPDKVCRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTSGGGSILAIYSTVASSLVLLVSLGAISF

SEQ ID NO: 57 - RNA, 1181 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 58 - RNA, 1005 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccucuggcggaggcagcauccuggccaucuacagcaccguggccagcagccuggugcugcuggugagccugggcgccaucagcuuc

SEQ ID NO: 59 - protein, 335 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTSGGGSILAIYSTVASSLVLLVSLGAISF

SEQ ID NO: 60 - RNA, 836 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagugauaauaggcuggagccucgguggccuagcuucuugccccuugggccuccccccagccccuccuccccuuccugcacccguacccccguggucuuugaauaaagucugagugggcggc

SEQ ID NO: 61 - RNA, 660 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaag

SEQ ID NO: 62 - protein, 220 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 63 - RNA, 1349 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagggaggaggcagcggcggcgauaucaucaagcuucugaacgagcaaguuaacaaggaaaugcagagcaguaaucucuacaugagcaugagcagcuggugcuacacccacucccuggacggagcaggccucuuccuguucgaccacgcagccgaggaguacgagcacgcuaagaaguugaucauuuucuugaacgagaacaacgugcccgugcagcuaacgucaaucagcgcaccugagcacaaguucgagggccugacccagaucuuccagaaggccuacgaacacgaacagcacaucuccgagagcaucaacaauauuguggaucacgcuaucaaguccaaggaccacgcuaccuucaacuuccugcagugguacguggccgagcaacaugaggaggaggugcuguucaaggacauccuggacaagaucgagcugaucgguaaugagaaucacggccuguaccuggccgaccaguacgugaagggcaucgccaagagccggaagucaggcucaugauaauaggcuggagccucgguggccuagcuucuugccccuugggccuccccccagccccuccuccccuuccugcacccguacccccguggucuuugaauaaagucugagugggcggc

SEQ ID NO: 64 - RNA, 1173 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagggaggaggcagcggcggcgauaucaucaagcuucugaacgagcaaguuaacaaggaaaugcagagcaguaaucucuacaugagcaugagcagcuggugcuacacccacucccuggacggagcaggccucuuccuguucgaccacgcagccgaggaguacgagcacgcuaagaaguugaucauuuucuugaacgagaacaacgugcccgugcagcuaacgucaaucagcgcaccugagcacaaguucgagggccugacccagaucuuccagaaggccuacgaacacgaacagcacaucuccgagagcaucaacaauauuguggaucacgcuaucaaguccaaggaccacgcuaccuucaacuuccugcagugguacguggccgagcaacaugaggaggaggugcuguucaaggacauccuggacaagaucgagcugaucgguaaugagaaucacggccuguaccuggccgaccaguacgugaagggcaucgccaagagccggaagucaggcuca

SEQ ID NO: 65 - protein, 391 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGGGSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 66 - RNA, 1376 nt, Artificial Sequence
  Synthetic
gggaaauaagagagaaaagaagaguaagaagaaauauaagaccccggcgccgccaccaugggcauccugcccagcccuggcaugcccgcucugcugagccuggugagccugcugagcgugcugcugaugggcugcguggcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggggcggaaguggaggcucugguggcagcggaggaucuggcggcggccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagugauaauaggcuggagccucgguggccuagcuucuugccccuugggccuccccccagccccuccuccccuuccugcacccguacccccguggucuuugaauaaagucugagugggcggc

SEQ ID NO: 67 - RNA, 1200 nt, Artificial Sequence
  Synthetic
augggcauccugcccagcccuggcaugcccgcucugcugagccuggugagccugcugagcgugcugcugaugggcugcguggcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggggcggaaguggaggcucugguggcagcggaggaucuggcggcggccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaag

SEQ ID NO: 68 - protein, 400 aa, Artificial Sequence
  Synthetic
MGILPSPGMPALLSLVSLLSVLLMGCVAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 69 - RNA, 1334 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagggaggaggcuccggaggcgguagcgcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcggugauaauaggcuggagccucgguggccuagcuucuugccccuugggccuccccccagccccuccuccccuuccugcacccguacccccguggucuuugaauaaagucugagugggcggc

SEQ ID NO: 70 - RNA, 1158 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagggaggaggcuccggaggcgguagcgcugagaccggcaugcagaucuacgagggcaagcugaccgcagagggccugcgguucggcaucguggccagccgcgccaaccacgcucugguggaccggcuuguggagggcgcuaucgacgccaucgugagacacggcggccgggaagaggacaucacccuggugcgggugugcggcagcugggagauucccgucgccgccggagaacuggcccggaaggaggacaucgacgccgugaucgccaucggcgugcugugcagaggcgccacgcccagcuucgacuacaucgccagcgaggugagcaagggccuggccgaccugagccuggagcugcggaagcccaucaccuucggcgugaucaccgccgacacccuggagcaggccaucgaggccgcaggcaccugccacggcaacaagggcugggaagccgcccugugcgccaucgagauggccaaccuguucaagagccugcgg

SEQ ID NO: 71 - protein, 386 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGGGSGGGSAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 72 - RNA, 947 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcugauaauaggcuggagccucgguggccuagcuucuugccccuugggccuccccccagccccuccuccccuuccugcacccguacccccguggucuuugaauaaagucugagugggcggc

SEQ ID NO: 73 - RNA, 771 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggc

SEQ ID NO: 74 - protein, 257 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGGSAIGGYIPEAPRDGQAYVRKDGEWVLLSTFLG

SEQ ID NO: 75 - RNA, 920 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 76 - RNA, 744 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 77 - protein, 248 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISF

SEQ ID NO: 78 - RNA, 1025 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 79 - RNA, 849 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcggaggcagcauccuggccaucuacagcaccguggccagcagccuggugcugcuggugagccugggcgccaucagcuuc

SEQ ID NO: 80 - protein, 283 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGGSAIGGYIPEAPRDGQAYVRKDGEWVLLSTFLGGGSILAIYSTVASSLVLLVSLGAISF

SEQ ID NO: 81 - RNA, 1028 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaagugauaauaggcuggagccucgguggccuagcuucuugccccuugggccuccccccagccccuccuccccuuccugcacccguacccccguggucuuugaauaaagucugagugggcggc

SEQ ID NO: 82 - RNA, 852 nt, Artificial Sequence
  Synthetic
auguacagcaugcagcuggcuagcugcgugacccugacccuggugcugcuggugaacagccagcccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaag

SEQ ID NO: 83 - protein, 284 aa, Artificial Sequence
  Synthetic
MYSMQLASCVTLTLVLLVNSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISFGGGSAIGGYIPEAPRDGQAYVRKDGEWVLLSTFLGK

SEQ ID NO: 84 - RNA, 1142 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 85 - RNA, 966 nt, Artificial Sequence
  Synthetic
auguacagcaugcagcuggcuagcugcgugacccugacccuggugcugcuggugaacagccagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuuc

SEQ ID NO: 86 - protein, 322 aa, Artificial Sequence
  Synthetic
MYSMQLASCVTLTLVLLVNSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSSGGGSILAIYSTVASSLVLLVSLGAISF

SEQ ID NO: 87 - RNA, 1142 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuuugccaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 88 - RNA, 966 nt, Artificial Sequence
  Synthetic
auguacagcaugcagcuggcuagcugcgugacccugacccuggugcugcuggugaacagccagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuuugccaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuuc

SEQ ID NO: 89 - protein, 322 aa, Artificial Sequence
  Synthetic
MYSMQLASCVTLTLVLLVNSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFCQFGRDIADTTDAVRDPQTLEILDITPCSSGGGSILAIYSTVASSLVLLVSLGAISF

SEQ ID NO: 90 - RNA, 1748 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 91 - RNA, 1572 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 92 - protein, 524 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISF

SEQ ID NO: 93 - RNA, 1925 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucaagaagaagaagcggccacggaacuccuacaagugcggcaccaacaccauggagcgggaggagagcgagcagaccaagaagcgggagaagauccacauuccugaacgguccgacgaagcccagcggguguucaagagcagcaagaccagcagcugcgacaagagcgacaccugcuucugauaauaggcuggagccucgguggccuagcuucuugccccuugggccuccccccagccccuccuccccuuccugcacccguacccccguggucuuugaauaaagucugagugggcggc

SEQ ID NO: 94 - RNA, 1749 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucaagaagaagaagcggccacggaacuccuacaagugcggcaccaacaccauggagcgggaggagagcgagcagaccaagaagcgggagaagauccacauuccugaacgguccgacgaagcccagcggguguucaagagcagcaagaccagcagcugcgacaagagcgacaccugcuuc

SEQ ID NO: 95 - protein, 583 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISFKKKKRPRNSYKCGTNTMEREESEQTKKREKIHIPERSDEAQRVFKSSKTSSCDKSDTCF

SEQ ID NO: 96 - RNA, 1805 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucaagcggcaguacaaggacaugaugagcgagggaggaccaccuggcgcugagccacagugauaauaggcuggagccucgguggccuagcuucuugccccuugggccuccccccagccccuccuccccuuccugcacccguacccccguggucuuugaauaaagucugagugggcggc

SEQ ID NO: 97 - RNA, 1629 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucaagcggcaguacaaggacaugaugagcgagggaggaccaccuggcgcugagccacag

SEQ ID NO: 98 - protein, 543 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISFKRQYKDMMSEGGPPGAEPQ

SEQ ID NO: 99 - RNA, 1853 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 100 - RNA, 1677 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcggaggcagcauccuggccaucuacagcaccguggccagcagccuggugcugcuggugagccugggcgccaucagcuuc

SEQ ID NO: 101 - protein, 559 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGGSAIGGYIPEAPRDGQAYVRKDGEWVLLSTFLGGGSILAIYSTVASSLVLLVSLGAISF

SEQ ID NO: 102 - RNA, 1856 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaagugauaauaggcuggagccucgguggccuagcuucuugccccuugggccuccccccagccccuccuccccuuccugcacccguacccccguggucuuugaauaaagucugagugggcggc

SEQ ID NO: 103 - RNA, 1680 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucggcggaggcagcgccaucggcggcuacauccccgaggccccuagagacggccaggccuacgugcggaaggacggcgagugggugcugcugagcaccuuccugggcaag

SEQ ID NO: 104 - protein, 560 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISFGGGSAIGGYIPEAPRDGQAYVRKDGEWVLLSTFLGK

SEQ ID NO: 105 - RNA, 1826 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagccugggcuucaucgccggccugaucgccaucgugauggugaccaucaugcugugcugcaugaccagcugcugcagcugccugaagggcuguugcagcugcggcagcugcugcaaguucgacgaggacgacugauaauaggcuggagccucgguggccuagcuucuugccccuugggccuccccccagccccuccuccccuuccugcacccguacccccguggucuuugaauaaagucugagugggcggc

SEQ ID NO: 106 - RNA, 1650 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagccugggcuucaucgccggccugaucgccaucgugauggugaccaucaugcugugcugcaugaccagcugcugcagcugccugaagggcuguugcagcugcggcagcugcugcaaguucgacgaggacgac

SEQ ID NO: 107 - protein, 550 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSLGFIAGLIAIVMVTIMLCCMTSCCSCLKGCCSCGSCCKFDEDD

SEQ ID NO: 108 - RNA, 1826 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcagcagcaucgccagcuucuucuucaucaucgggcugaucaucggccucuuccuggugcugcgggugggcauccaccugugcaucaagcugaagcacaccaagaagagacagaucuacaccgacaucgagaugaaccggcugggcaagugauaauaggcuggagccucgguggccuagcuucuugccccuugggccuccccccagccccuccuccccuuccugcacccguacccccguggucuuugaauaaagucugagugggcggc

SEQ ID NO: 109 - RNA, 1650 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcagcagcaucgccagcuucuucuucaucaucgggcugaucaucggccucuuccuggugcugcgggugggcauccaccugugcaucaagcugaagcacaccaagaagagacagaucuacaccgacaucgagaugaaccggcugggcaag

SEQ ID NO: 110 - protein, 550 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSSSIASFFFIIGLIIGLFLVLRVGIHLCIKLKHTKKRQIYTDIEMNRLGK

SEQ ID NO: 111 - RNA, 1775 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggcugauaauaggcuggagccucgguggccuagcuucuugccccuugggccuccccccagccccuccuccccuuccugcacccguacccccguggucuuugaauaaagucugagugggcggc

SEQ ID NO: 112 - RNA, 1599 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcggcagcgccaucggcggcuacauccccgaggccccuagagacggccaggccuacgugcggaaggacggcgagugggugcugcugagcaccuuccugggc

SEQ ID NO: 113 - protein, 533 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGGSAIGGYIPEAPRDGQAYVRKDGEWVLLSTFLG

SEQ ID NO: 114 - RNA, 1973 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 115 - RNA, 1797 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuuc

SEQ ID NO: 116 - protein, 599 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSSGGGSILAIYSTVASSLVLLVSLGAISF

SEQ ID NO: 117 - RNA, 1799 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 118 - RNA, 1623 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 119 - protein, 541 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISF

SEQ ID NO: 120 - RNA, 2024 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 121 - RNA, 1848 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcugcuggugagccugggcgccaucagcuuc

SEQ ID NO: 122 - protein, 616 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTGGGSGGGQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSSGGGSILAIYSTVASSLVLLVSLGAISF

SEQ ID NO: 123 - RNA, 3995 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacaagguggaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 124 - RNA, 3819 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacaagguggaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 125 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 126 - protein, 5 aa, Artificial Sequence
  Synthetic
KPSKR

SEQ ID NO: 127 - protein, 10 aa, Artificial Sequence
  Synthetic
TQTNSPRRAR

SEQ ID NO: 128 - RNA, 9 nt, Artificial Sequence
  Synthetic
  misc_feature (3)..(3): R is A or G
ccrccaugg

SEQ ID NO: 129 - RNA, 11 nt, Artificial Sequence
  Synthetic
gggauccuacc

SEQ ID NO: 130 - RNA, 9 nt, Artificial Sequence
  Synthetic
  misc_feature (8)..(9): N is U or A
uuauuuann

SEQ ID NO: 131 - RNA, 47 nt, Artificial Sequence
  Synthetic
gggaaauaagagagaaaagaagaguaagaagaaauauaagagccacc

SEQ ID NO: 132 - RNA, 119 nt, Artificial Sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 133 - protein, 15 aa, Artificial Sequence
  Synthetic
GGSGGSGGSGGSGGG

SEQ ID NO: 134 - protein, 8 aa, Artificial Sequence
  Synthetic
GGGSGGGS

SEQ ID NO: 135 - protein, 7 aa, Artificial Sequence
  Synthetic
GGGSGGG

SEQ ID NO: 136 - protein, 4 aa, Artificial Sequence
  Synthetic
  MISC_FEATURE (1)..(4): may be repeated 1-5 times
GGGS

SEQ ID NO: 137-144 - skipped (8 entries)

SEQ ID NO: 145 - RNA, 1785 nt, Artificial Sequence
  Synthetic
auguacagcaugcagcuggcuagcugcgugacccugacccuggugcugcuggugaacagccagggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 146 - protein, 595 aa, Artificial Sequence
  Synthetic
MYSMQLASCVTLTLVLLVNSQGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 147 - RNA, 1961 nt, Artificial Sequence
  Synthetic
gggaaauaagagagaaaagaagaguaagaagaaauauaagaccccggcgccgccaccauguacagcaugcagcuggcuagcugcgugacccugacccuggugcugcuggugaacagccagggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacccucccgaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 148 - protein, 13 aa, Artificial Sequence
  Synthetic
AKFVAAWTLKAAA

SEQ ID NO: 149 - RNA, 2216 nt, Artificial Sequence; identical to SEQ ID NO: 1
  Synthetic

SEQ ID NO: 150 - RNA, 2040 nt, Artificial Sequence; identical to SEQ ID NO: 3
  Synthetic

SEQ ID NO: 151 - protein, 680 aa, Artificial Sequence; identical to SEQ ID NO: 5
  Synthetic

SEQ ID NO: 152 - protein, 4 aa, Artificial Sequence
  Synthetic
  MISC_FEATURE (1)..(4): may be repeated 1-5 times
GSGG

(19) United States
(12) Patent Application Publication
(10) Pub. No.: US 2024/0299531 A1
(43) Pub. Date: Sep. 12, 2024

(54) THERAPEUTIC USE OF SARS-COV-2 MRNA DOMAIN VACCINES

(71) Applicant: Moderna TX, Inc, Cambridge, MA (US)

(72) Inventor: Guillaume Stewart-Jones, Cambridge, MA (US)

(73) Assignee: Moderna TX, Inc, Cambridge, MA (US)

(21) Appl. No.: 18/282,097

(22) PCT Filed: Mar. 14, 2022
(86) PCT No.: PCT/US2022/020218

§ 371 Date: Sep. 14, 2023

Related U.S. Application Data
(60) Provisional application No. 63/281,021, filed on Nov. 18, 2021.
(60) Provisional application No. 63/161,429, filed on Mar. 15, 2021.

(51) Int. Cl. A61K 39/215; A61K 9/51; A61K 39/00; A61P 37/04

(52) U.S. Cl. CPC A61K 39/215; A61K 9/5123; A61P 37/04; A61K 2039/53; A61K 2039/54

(57) ABSTRACT

The disclosure describes coronavirus ribonucleic acid (RNA) vaccines as well as methods of using the vaccines and compositions comprising the vaccines. The RNA vaccines encode domains and subunits of coronavirus.

### RELATED APPLICATIONS

This application claims the benefit under 35 U.S.C. § 119(e) of U.S. provisional application No. 63/161,429, filed Mar. 15, 2021 and U.S. provisional application No. 63/281,021, filed Nov. 18, 2021, each of which is incorporated by reference herein in its entirety.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Mar. 14, 2022, is named M137870175WO00-SEQ-JXV and is 432,622 bytes in size.

### BACKGROUND

Human coronaviruses are highly contagious enveloped, positive single-stranded RNA viruses of the Coronaviridae family. Two sub-families of Coronaviridae are known to cause human disease. The most important being the β-coronaviruses (betacoronaviruses). Several previously known β-coronaviruses are common etiological agents of mild to moderate upper respiratory tract infections. However, in recent years there have been successive outbreaks of more lethal coronaviruses, such as SARS and MERS. The most recent novel coronavirus was initially identified from the Chinese city of Wuhan in December 2019 and has been associated with a high mortality rate. This recently identified coronavirus, referred to as Severe Acute Respiratory Syndrome Coronavirus 2 (SARS-CoV-2) (formerly referred to as a “2019 novel coronavirus,” or a “2019-nCoV”) has rapidly infected millions of people and caused a global pandemic. The pandemic disease that the SARS-CoV-2 virus causes has been named by World Health Organization (WHO) as COVID-19 (Coronavirus Disease 2019). The first genome sequence of a SARS-CoV-2 isolate (Wuhan-Hu-1; USA-WA1/2020 isolate) was released by investigators from the Chinese CDC in Beijing on Jan. 10, 2020 at Virological, a UK-based discussion forum for analysis and interpretation of virus molecular evolution and epidemiology. The sequence was then deposited in GenBank on Jan. 12, 2020, having Genbank Accession number MN908947.1.

The first treatments and vaccines were based on the initial strain first sequenced in Wuhan, China. However, during the pandemic, virus evolution has remained high and vaccines must evolve to meet the changing nature of the pandemic. The continuing health problems and mortality associated with coronavirus infections, particularly the SARS-CoV-2 pandemic, are of tremendous concern internationally. The public health crisis caused by SARS-CoV-2 reinforces the importance of rapidly developing effective and safe vaccine candidates against these viruses.

### SUMMARY

Provided herein, in some embodiments, are methods of administering a therapeutic dose of compositions (e.g., vaccines) that comprise a messenger ribonucleic acid (mRNA) that encodes highly immunogenic antigen(s) capable of eliciting potent neutralizing antibody responses against SARS-CoV-2 antigens. The mRNA molecules described herein are used to express key neutralizing domains of the SARS-CoV-2 coronavirus spike (S) protein that are efficient at inducing protective immunity when used individually or in combination as an immunogenic composition or vaccine to protect people from infection by the natural virus and/or to reduce symptoms if infected.

The envelope S proteins of known betacoronaviruses determine the virus host tropism and entry into host cells and are critical for SARS-CoV-2 infection. The organization of the S protein is similar among betacoronaviruses, such as SARS-CoV-2, SARS-CoV, MERS-CoV, HKU1-CoV, MHV-CoV and NL63-CoV, including two subunits, S1 and S2, which mediate attachment and membrane fusion, respectively. The S1 subunit includes an N terminal domain (NTD) and a receptor binding domain (RBD).

The expression of subunit antigens focuses the immune response to specific subunits with minimal stimulation of memory B and T cells specific to other domains of the antigen that are shared with other related viruses.

Additionally, both the NTD and RBD are known to be sites for binding of antibodies that neutralize virus activity. RBD in the case of SARS-CoV-2 is the receptor binding site of the spike protein which binds the angiotensin-converting enzyme 2 (ACE2). The NTD, the function of which is not thoroughly understood, seems to have a role in binding sugar moieties and in facilitating the conformational transition of the spike protein from prefusion to a post fusion conformation. Regardless, both the NTD and RBD domains induce high binding antibody and neutralizing antibody titers.

Thus, some aspects of the present disclosure provide methods of administering to a human subject a therapeutic dose of a composition comprising a messenger ribonucleic acid (mRNA) comprising an open reading frame (ORF) that encodes a fusion protein comprising at least two domains of a SARS-CoV-2 Spike (S) protein, and less than the full length spike protein, wherein the mRNA is in a lipid nanoparticle.

In some embodiments, the therapeutic dose is 2.5 μg of the composition. In some embodiments, the therapeutic dose is 5 μg of the composition. In some embodiments, the therapeutic dose is 30 μg of the composition. In some embodiments, the therapeutic dose is 10 μg of the composition. In some embodiments, the the therapeutic dose is 10 μg-30 μg of the composition. In some embodiments, the therapeutic dose is 10 μg-20 μg of the composition. In some embodiments, the therapeutic dose is at least 10 μg and less than 25 μg of the composition. In some embodiments, the therapeutic dose is 5 μg-30 μg of the composition. In some embodiments, the therapeutic dose is at least 5 μg and less than 25 μg of the composition. In some embodiments, the therapeutic dose is 100 μg of the composition. In some embodiments, the method comprises administering to the subject at least two doses of the composition (e.g., two 10 μg doses, two 30 μg doses). In some embodiments, a second dose of the composition is administered to the subject at least 28 days after a first dose of the composition is administered to the subject and within one year of the first dose.

In some embodiments, the composition further comprises Tris buffer. In some embodiments, the Tris buffer comprises sucrose, and sodium acetate. In some embodiments, the composition comprises 10 mM-30 mM Tris buffer comprising 75 mg/mL-95 mg/mL sucrose, and 5 mM-15 mM sodium acetate, optionally wherein the composition has a pH of 6-8. In some embodiments, the composition comprises 20 mM Tris buffer comprising 87 mg/mL sucrose, and 10.7 mM sodium acetate, optionally wherein the composition has a pH of 7.5.

In some embodiments, the composition comprises 0.5 mg/mL of the mRNA.

In some embodiments, the composition is administered intramuscularly, optionally into a deltoid muscle of the subject's arm.

In some embodiments, the fusion protein comprises an amino acid sequence having at least 90%, at least 95%, or at least 98% identity to the sequence of SEQ ID NO: 92. In some embodiments, the fusion protein comprises the amino acid sequence of SEQ ID NO: 92.

In some embodiments, the ORF comprises a nucleotide sequence having at least 90%, at least 95%, or at least 98% identity to the sequence of SEQ ID NO: 91. In some embodiments, the ORF comprises the nucleotide sequence of SEQ ID NO: 91.

In some embodiments, the mRNA comprises a nucleotide sequence having at least 90%, at least 95%, or at least 98% identity to the sequence of SEQ ID NO: 90. In some embodiments, the mRNA comprises the nucleotide sequence of SEQ ID NO: 90.

In some embodiments, the lipid nanoparticle comprises: ionizable amino lipid; neutral lipid; sterol; and PEG-modified lipid. In some embodiments, the lipid nanoparticle comprises: 40-55 mol % ionizable amino lipid; 5-15 mol % neutral lipid; 35-45 mol % sterol; and 1-5 mol % PEG-modified lipid. In some embodiments, the lipid nanoparticle comprises: 47 mol % ionizable amino lipid; 11.5 mol % neutral lipid; 38.5 mol % sterol; and 3.0 mol % PEG-modified lipid; 48 mol % ionizable amino lipid; 11 mol % neutral lipid; 38.5 mol % sterol; and 2.5 mol % PEG-modified lipid; 49 mol % ionizable amino lipid; 10.5 mol % neutral lipid; 38.5 mol % sterol; and 2.0 mol % PEG-modified lipid; 50 mol % ionizable amino lipid; 10 mol % neutral lipid; 38.5 mol % sterol; and 1.5 mol % PEG-modified lipid; or 51 mol % ionizable amino lipid; 9.5 mol % neutral lipid; 38.5 mol % sterol; and 1.0 mol % PEG-modified lipid.

In some embodiments, the ionizable amino lipid is heptadecan-9-yl 8 ((2 hydroxyethyl)(6 oxo 6-(undecyloxy)hexyl)amino)octanoate (Compound 1). In some embodiments, the neutral lipid is 1,2 distearoyl sn glycero-3 phosphocholine (DSPC). In some embodiments, the sterol is cholesterol. In some embodiments, the PEG-modified lipid is 1-monomethoxypolyethyleneglycol-2,3-dimyristylglycerol with polyethylene glycol of average molecular weight 2000 (PEG2000 DMG).

In some embodiments, the age of the subject is 18 to 54 years or 55 years or older. In some embodiments, the subject is immunocompromised. In some embodiments, the subject has a chronic pulmonary disease, such as chronic obstructive pulmonary disease (COPD) or asthma.

In some embodiments, the subject has an underlying comorbid condition, optionally selected from obesity, heart disease, diabetes, and lung disease.

Some aspects of the disclosure provide a composition comprising a dose of an mRNA encoding a domain of a SARS-CoV-2 Spike protein in a lipid nanoparticle, wherein the dose is at least 5 μg and less than 20 μg. In some embodiments, the dose is 10 μg.

Some aspects of the disclosure provide a composition comprising a 30 μg dose of an mRNA encoding a domain of a SARS-CoV-2 Spike protein in a lipid nanoparticle. In some embodiments, the domain comprises an amino (N)-terminal domain of a SARS-CoV-2 Spike protein. In some embodiments, the domain comprises a receptor binding domain of a SARS-CoV-2 Spike protein.

Some aspects of the disclosure provide a method comprising administering to a human subject a therapeutic dose of a composition described herein in an effective amount to produce an immune response against the domain of the SARS-CoV-2 Spike protein.

In some embodiments, the geometric mean titer (GMT) of neutralizing antibody titers induced against SARS-CoV-2 (D614G) in the subject at least 45 days post administration of two doses, optionally 45 to 100 days post-administration of two doses, is at least 1,000. In some embodiments, the GMT of neutralizing antibody titers induced against SARS-CoV-2 (B.1.351) in the subject at least 45 days post administration of two doses, optionally 45 to 100 days post-administration of two doses, is at least 110.

In some embodiments, the geometric mean ratio (GMR) of neutralizing antibody titers induced against SARS-CoV-2 (B.1.351) in the subject at least 45 days post administration of two doses, optionally 45 to 100 days post-administration of two doses, relative to a neutralizing antibody titer induced against SARS-CoV-2 (B.1.351) in a second subject administered mRNA encoding a SARS-CoV-2 spike protein comprising a double proline stabilizing mutation is at least 1.05.

In some embodiments, the geometric mean fold rise (GMFR) of neutralizing antibody titers induced against SARS-CoV-2 (D614G) in the subject at least 45 days post administration of two doses, optionally 45 to 100 days post-administration of two doses, relative to a neutralizing antibody titer induced against SARS-CoV-2 (D614G) in the subject prior to administration of the composition is at least 90. In some embodiments, the GMFR of neutralizing antibody titers induced against SARS-CoV-2 (B.1.351) in the subject at least 45 days post administration of two doses, optionally 45 to 100 days post-administration of two doses, relative to a neutralizing antibody titer induced against SARS-CoV-2 (B.1.351) in the subject prior to administration of the composition is at least 8.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 Schematic representation of wild-type and 2P spike protein antigens encoded by mRNAs of the invention; signal peptide (SP), no fill, N-terminal domain (NTD), dotted; receptor-binding domain (RBD), downward diagonal stripes; subdomain 1 (SD1), horizontal stripes; subdomain 2 (SD2), wave; fusion peptide (FP), upward diagonal stripes; heptad repeat 1 (HR1) weave; heptad repeat 2 (HR2) diagonal brick; (TM), vertical stripes; and cytoplasmic tail (CT), brick.

FIG. 2 Data showing mRNA-1283 (mRNA encoding a fusion protein comprising a first domain comprising an amino (N)-terminal domain of a SARS-CoV-2 Spike protein and a second domain comprising a receptor binding domain of a SARS-CoV-2 Spike protein; SEQ ID NO: 90; ORF SEQ ID NO: 91; encoding SEQ ID NO: 92) (two doses) elicits comparable levels of prototype pseudovirus neutralizing antibodies compared to mRNA-1273 (SEQ ID NO: 123; ORF SEQ ID NO: 124; encoding SEQ ID NO: 125) at all dose levels.

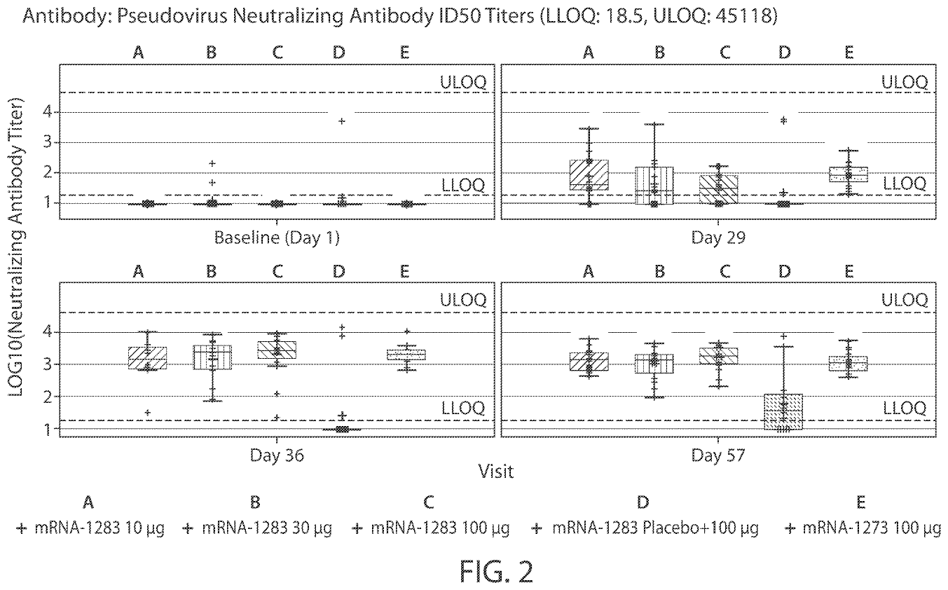

FIG. 3 Data showing mRNA-1283 (two doses) elicits comparable levels of B.1.351 neutralizing antibodies compared to 1273 at all dose levels.

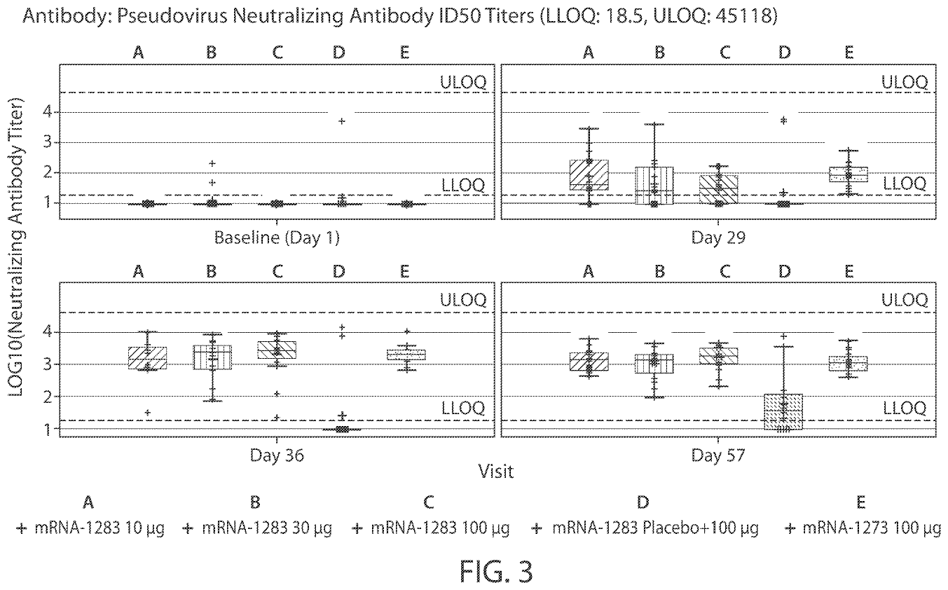

FIG. 4 Data showing mRNA-1283 (two doses) elicits comparable levels of wild-type spike binding antibodies compared 1273 at all dose levels.

FIG. 5 Data showing mRNA-1283 (two doses) elicits comparable levels of wild-type RBD binding antibodies compared 1273 at all dose levels.

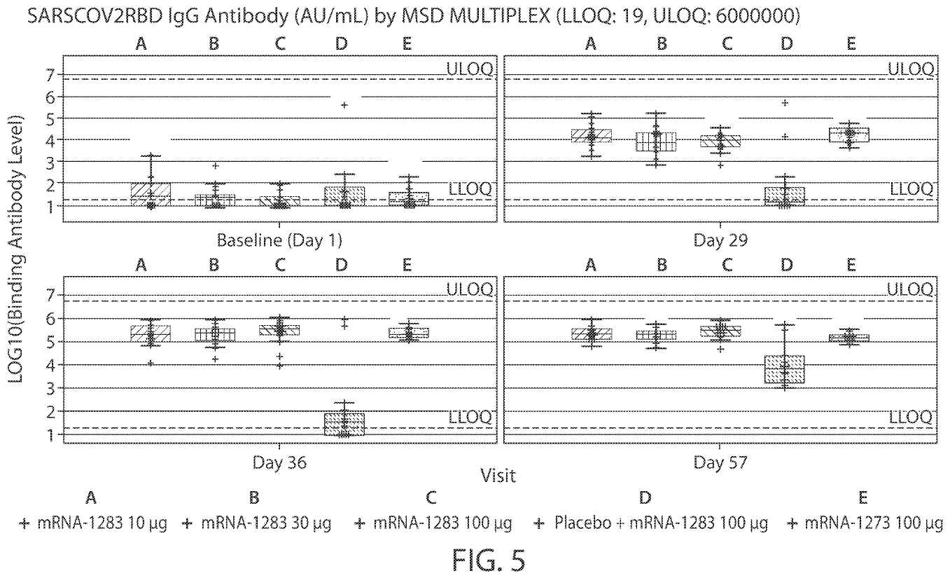

### DETAILED DESCRIPTION

Severe acute respiratory syndrome coronavirus 2 (SARS-CoV-2) is a newly emerging respiratory virus with high morbidity and mortality. SARS-CoV-2 has rapidly spread around the world compared with SARS-CoV, which appeared in 2002, and Middle East respiratory syndrome coronavirus (MERS-CoV), which emerged in 2012. The World Health Organization (WHO) reports that, as of March 2021, the current outbreak of COVID-19 has had over 120 million confirmed cases worldwide with more than 2.65 million deaths. New cases of COVID-19 infection are on the rise and are still increasing rapidly. It is thus crucial that a variety of safe and effective vaccines and drugs be developed to prevent and treat COVID-19 and reduce the serious impact that COVID-19 is having across the world. Vaccines and drugs made using a variety of modalities, and vaccines having improved safety and efficacy, are imperative. There remains a need to accelerate the advanced design and development of vaccines and therapeutic drugs against coronavirus disease 2019 (COVID-19).

On Jan. 7, 2020, severe acute respiratory syndrome coronavirus 2 (SARS-CoV-2) was identified as the etiological agent of a novel pneumonia that emerged in December 2019, in Wuhan City, Hubei province in China (Lu H. et al. (2020) J Med Virol. April; 92(4):401-402.). Soon after, the virus caused an outbreak in China and has spread to the world. According to the analysis of genomic structure of SARS-CoV-2, it belongs to β-coronaviruses (CoVs) (Chan et al. 2020 Emerg Microbes Infect.; 9(1):221-236).

A key protein on the surface of coronavirus is the Spike protein. A large variety of mRNA constructs have been designed and are disclosed herein. When formulated in appropriate delivery vehicles mRNA encoding Spike antigen, subunits and domains thereof are capable of inducing a strong immune response against SARS-CoV-2, thus producing effective and potent mRNA vaccines. Administration of the mRNA encoding various Spike protein antigens, in particular, Spike protein subunit and domain antigens, results in delivery of the mRNA to immune tissues and cells of the immune system where it is rapidly translated into proteins antigens. Other immune cells, for example, B cells and T cells, are then able to recognize and mount and immune response develop an immune response against the encoded protein and ultimately create a long-lasting protective response against the coronavirus. Low immunogenicity, a drawback in protein vaccine development due to poor presentation to the immune system or incorrect folding of the antigens, is avoided through the use of the highly effective mRNA vaccines encoding spike protein, subunits and domains thereof disclosed herein.

The present disclosure provides compositions (e.g., mRNA vaccines) that elicit potent neutralizing antibodies against coronavirus antigens. In some embodiments, a composition includes mRNA encoding at least one (e.g., one, two, or more) coronavirus antigen, such as a SARS-CoV-2 antigen. In some embodiments, the mRNA encodes a spike protein domain, such as a receptor binding domain (RBD), an N-terminal domain (NTD), or a combination of an RBD and NTD.

The present disclosure provides vaccine compositions and vaccination methods that elicit potent neutralizing antibodies against coronavirus antigens at low doses, previously considered in some instances to be sub-therapeutic. In some embodiments, a composition includes messenger RNA (mRNA) encoding a fusion protein comprising at least two domains of a SARS-CoV-2 Spike (S) protein, and less than the full length spike protein in a lipid nanoparticle (LNP). In some embodiments, the fusion protein comprises the domains associated with inducing high titers of neutralizing antibodies such as the N-terminal domain (NTD) and the receptor binding domain (RBD).

Provided herein are compositions (e.g., vaccine compositions) for inducing a neutralizing antibody response to SARS-CoV-2 Spike (S) protein in subject. The compositions provided herein can be used therapeutically or prophylactically. The compositions provided herein comprise a messenger ribonucleic acid (mRNA) comprising an open reading frame (ORF) that encodes a fusion protein comprising at least two domains of a SARS-CoV-2 Spike (S) protein, such as the NTD and RBD domains and less than the full length spike protein, wherein the mRNA is in a lipid nanoparticle.

Both the NTD and RBD are known to be sites for binding of antibodies that neutralize virus activity. RBD in the case of SARS-CoV-2 is the receptor binding site of the spike protein which binds the angiotensin-converting enzyme 2 (ACE2). The NTD, the function of which is not thoroughly understood, seems to have a role in binding sugar moieties and in facilitating the conformational transition of the spike protein from prefusion to a post fusion conformation. Both the NTD and RBD domains induce high binding antibody and neutralizing antibody titers at the low doses disclosed herein.

For example therapeutic doses of mRNA encoding fusion protein of a membrane bound RBD antigen (RBD-TM) or a membrane bound NTD antigen (NTD-TM) are used herein to produce an immune response to the SARS-CoV-2 S1/S2 spike protein. A therapeutic dose as used herein is a dose which is sufficient to induce, or boost the presence of, neutralizing antibodies in a subject. Such a dose can be administered to seropositive or seronegative subjects. For example, a subject may be naïve and not have antibodies that react with SARS-CoV-2 or may have preexisting antibodies to SARS-CoV-2 because the subject has previously had an infection with SARS-CoV-2 or may have previously been administered a dose of a vaccine (e.g., an mRNA vaccine) that induces antibodies against SARS-CoV-2. In some embodiments the therapeutic dose is a dose of 2.5 μg to 100 μg or any integer dose therebetween. In some embodiments, quite surprisingly, the therapeutic dose is a low dose not previously demonstrated to be sufficient to induce a neutralizing antibody response in a human subject, such as, for instance, 2.5 μg to less than 35 μg or 5 μg to less than 25 μg or any integer therebetween. In some embodiments the dose is 2.5 μg-30 μg, 2.5 μg-25 μg, 2.5 μg-20 μg, 2.5 μg-2.15 μg, 2.5 μg-10 μg, 2.5 μg-5 μg, 5 μg-30 μg, 5 μg-25 μg, 5 μg-20 μg, 5 μg-15 μg, 5 μg-10 μg, 10 μg-30 μg, 10 μg-25 μg, 10 μg-20 μg, about 2.5 μg, about 5 μg, about 10 μg or about 30 μg. In some embodiments, the therapeutic dose is the first dose administered to the subject, the second dose administered to the subject or both the first and second dose administered to the subject.

Some aspects of the present disclosure provide a messenger ribonucleic acid (mRNA) comprising an open reading frame encoding a fusion protein comprising a receptor binding domain (RBD) of a SARS-CoV-2 Spike protein and a protein transmembrane domain, e.g., a naturally occurring or heterologous transmembrane domain.

In some embodiments, the protein transmembrane domain is an influenza hemagglutinin transmembrane domain.

In some embodiments, the fusion protein comprises an amino acid sequence having at least 80% identity to the amino acid sequence of SEQ ID NO: 77.

In some embodiments, the fusion protein comprises an amino acid sequence having at least 85%, at least 90%, at least 95%, at least 96%, at least 97%, at least 98%, or at least 99% identity to the amino acid sequence of SEQ ID NO: 77.

In some embodiments, the fusion protein comprises the amino acid sequence of SEQ ID NO: 77.

In some embodiments, the open reading frame comprises a nucleotide sequence having at least 70% identity to the nucleotide sequence of SEQ ID NO: 76.

In some embodiments, the wherein the open reading frame comprises a nucleotide sequence having at least 75%, at least 80%, at least 90%, at least 95%, at least 96%, at least 97%, at least 98%, or at least 99% identity to the nucleotide sequence of SEQ ID NO: 76.

In some embodiments, the open reading frame comprises the nucleotide sequence of SEQ ID NO: 76.

Other aspects of the present disclosure provide a messenger ribonucleic acid (mRNA) comprising an open reading frame encoding a fusion protein comprising an amino (N)-terminal domain of a SARS-CoV-2 Spike protein and a transmembrane domain.

In some embodiments, the transmembrane domain is an influenza hemagglutinin transmembrane domain.

In some embodiments, the fusion protein comprises an amino acid sequence having at least 80% identity to the amino acid sequence of SEQ ID NO: 47.

In some embodiments, the fusion protein comprises an amino acid sequence having at least 85%, at least 90%, at least 95%, at least 96%, at least 97%, at least 98%, or at least 99% identity to the amino acid sequence of SEQ ID NO: 47.

In some embodiments, the fusion protein comprises the amino acid sequence of SEQ ID NO: 47.

In some embodiments, the open reading frame comprises a nucleotide sequence having at least 70% identity to the nucleotide sequence of SEQ ID NO: 46.

In some embodiments, the open reading frame comprises a nucleotide sequence having at least 75%, at least 80%, at least 90%, at least 95%, at least 96%, at least 97%, at least 98%, or at least 99% identity to the nucleotide sequence of SEQ ID NO: 46.

In some embodiments, the open reading frame comprises the nucleotide sequence of SEQ ID NO: 46.

Yet other aspects of the present disclosure provide a messenger ribonucleic acid (mRNA) comprising an open reading frame encoding a fusion protein comprising a receptor binding domain of a SARS-CoV-2 Spike protein linked to an amino (N)-terminal domain of a SARS-CoV-2 Spike protein, optionally via a linker.

In some embodiments, the fusion protein further comprises a transmembrane domain.

In some embodiments, the fusion protein comprises an amino acid sequence having at least 80% identity to the amino acid sequence of SEQ ID NO: 92.

In some embodiments, the fusion protein comprises an amino acid sequence having at least 85%, at least 90%, at least 95%, at least 96%, at least 97%, at least 98%, or at least 99% identity to the amino acid sequence of SEQ ID NO: 92.

In some embodiments, the fusion protein comprises the amino acid sequence of SEQ ID NO: 92.

In some embodiments, the open reading frame comprises a nucleotide sequence having at least 70% identity to the nucleotide sequence of SEQ ID NO: 91.

In some embodiments, the open reading frame comprises a nucleotide sequence having at least 75%, at least 80%, at least 90%, at least 95%, at least 96%, at least 97%, at least 98%, or at least 99% identity to the nucleotide sequence of SEQ ID NO: 91.

In some embodiments, the open reading frame comprises the nucleotide sequence of SEQ ID NO: 91.

In some embodiments, the mRNA further comprises a 5′ untranslated region (UTR), optionally comprising the nucleotide sequence of SEQ ID NO: 131 or 2.

In some embodiments, the mRNA further comprises a 3′ untranslated region (UTR), optionally comprising the nucleotide sequence of SEQ ID NO: 132 or 4.

In some embodiments, the mRNA further comprises a 5′ cap, optionally 7mG(5′)ppp(5′)NlmpNp.

In some embodiments, the mRNA further comprises a polyA tail, optionally having a length of about 100 nucleotides.

In some embodiments, the mRNA comprises a chemical modification, optionally 1-methylpseudouridine.

Some aspects of the present disclosure provide a composition comprising the mRNA of any one of the preceding paragraphs.

Other aspects of the present disclosure provide a composition comprising at least two of the mRNA of any one of the preceding paragraphs.

Other aspects of the present disclosure provide a composition comprising: (a) a messenger ribonucleic acid (mRNA) comprising an open reading frame encoding a fusion protein comprising a receptor binding domain (RBD) of a SARS-CoV-2 Spike protein and a protein transmembrane domain; and (b) an mRNA comprising an open reading frame encoding a fusion protein comprising an amino (N)-terminal domain of a SARS-CoV-2 Spike protein and a transmembrane domain. In some embodiments, the ratio of the mRNA of (a) to the mRNA of (b) is about 1:1, e.g., 1:2, 1:3, 21:, or 3:1. In some embodiments, at least 50% of the mRNA of a composition is the mRNA of (a). For example, at least 55%, 60%, 65%, 70%, 75%, 80%, 85%, 90%, or 95% of the mRNA of a composition is the mRNA of (a). In some embodiments, at least 50% of the mRNA of a composition is the mRNA of (b). For example, at least 55%, 60%, 65%, 70%, 75%, 80%, 85%, 90%, or 95% of the mRNA of a composition is the mRNA of (b).

In some embodiments, the protein transmembrane domain is an influenza hemagglutinin transmembrane domain.

In some embodiments, the fusion protein of (a) comprises an amino acid sequence having at least 80% identity to the amino acid sequence of SEQ ID NO: 77.

In some embodiments, the fusion protein of (a) comprises an amino acid sequence having at least 85%, at least 90%, at least 95%, at least 96%, at least 97%, at least 98%, or at least 99% identity to the amino acid sequence of SEQ ID NO: 77.

In some embodiments, the fusion protein of (a) comprises the amino acid sequence of SEQ ID NO: 77.

In some embodiments, the open reading frame of (a) comprises a nucleotide sequence having at least 70% identity to the nucleotide sequence of SEQ ID NO: 76.

In some embodiments, the open reading frame of (a) comprises a nucleotide sequence having at least 75%, at least 80%, at least 90%, at least 95%, at least 96%, at least 97%, at least 98%, or at least 99% identity to the nucleotide sequence of SEQ ID NO: 76.

In some embodiments, the open reading frame of (a) comprises the nucleotide sequence of SEQ ID NO: 76.

In some embodiments, the fusion protein of (b) comprises an amino acid sequence having at least 80% identity to the amino acid sequence of SEQ ID NO: 47.

In some embodiments, the fusion protein of (b) comprises an amino acid sequence having at least 85%, at least 90%, at least 95%, at least 96%, at least 97%, at least 98%, or at least 99% identity to the amino acid sequence of SEQ ID NO: 47.

In some embodiments, the fusion protein of (b) comprises the amino acid sequence of SEQ ID NO: 47.

In some embodiments, the open reading frame of (b) comprises a nucleotide sequence having at least 70% identity to the nucleotide sequence of SEQ ID NO: 46.

In some embodiments, the open reading frame of (b) comprises a nucleotide sequence having at least 75%, at least 80%, at least 90%, at least 95%, at least 96%, at least 97%, at least 98%, or at least 99% identity to the nucleotide sequence of SEQ ID NO: 46.

In some embodiments, the open reading frame of (b) comprises the nucleotide sequence of SEQ ID NO: 46.

In some embodiments, the mRNA is formulated in a lipid nanoparticle.

In some embodiments, the composition further comprises a lipid nanoparticle.

In some embodiments, the mRNA of (a) is formulated in a lipid nanoparticle, and the mRNA of (b) is formulated in a lipid nanoparticle.

In some embodiments, the lipid nanoparticle comprises a cationic lipid. In some embodiments, the cationic lipid is an ionizable amino lipid.

In some embodiments, the lipid nanoparticle further comprises a neutral lipid.

In some embodiments, the lipid nanoparticle further comprises a sterol.

In some embodiments, the lipid nanoparticle further comprises a polyethylene glycol (PEG)-modified lipid.

In some embodiments, the lipid nanoparticle comprises an ionizable amino lipid, a neutral lipid, a sterol, and a PEG-modified lipid.

In some embodiments, the ionizable amino lipid is heptadecan-9-yl 8 ((2 hydroxyethyl)(6 oxo 6-(undecyloxy)hexyl)amino)octanoate (Compound 1).

In some embodiments, the neutral lipid is 1,2 distearoyl-sn-glycero-3-phosphocholine (DSPC).

In some embodiments, the sterol is cholesterol.

In some embodiments, the PEG-modified lipid is 1,2 dimyristoyl-sn-glycerol, methoxypolyethyleneglycol (PEG2000 DMG).

In some embodiments, the lipid nanoparticle comprises 20-60 mol % ionizable amino lipid, 5-25 mol % neutral lipid, 25-55 mol % sterol, and 0.5-15 mol % PEG-modified lipid.

In some embodiments, the lipid nanoparticle comprises: 47 mol % ionizable amino lipid; 11.5 mol % neutral lipid; 38.5 mol % sterol; and 3.0 mol % PEG-modified lipid; 48 mol % ionizable amino lipid; 11 mol % neutral lipid; 38.5 mol % sterol; and 2.5 mol % PEG-modified lipid; 49 mol % ionizable amino lipid; 10.5 mol % neutral lipid; 38.5 mol % sterol; and 2.0 mol % PEG-modified lipid; 50 mol % ionizable amino lipid; 10 mol % neutral lipid; 38.5 mol % sterol; and 1.5 mol % PEG-modified lipid; or 51 mol % ionizable amino lipid; 9.5 mol % neutral lipid; 38.5 mol % sterol; and 1.0 mol % PEG-modified lipid.

In some embodiments, the lipid nanoparticle comprises: 47 mol % Compound 1; 11.5 mol % DSPC; 38.5 mol % cholesterol; and 3.0 mol % PEG2000 DMG; 48 mol % Compound 1; 11 mol % DSPC; 38.5 mol % cholesterol; and 2.5 mol % PEG2000 DMG; 49 mol % Compound 1; 10.5 mol % DSPC; 38.5 mol % cholesterol; and 2.0 mol % PEG2000 DMG; 50 mol % Compound 1; 10 mol % DSPC; 38.5 mol % cholesterol; and 1.5 mol % PEG2000 DMG; or 51 mol % Compound 1; 9.5 mol % DSPC; 38.5 mol % cholesterol; and 1.0 mol % PEG2000 DMG.

Further aspects of the present disclosure provide a method comprising administering to a subject the mRNA or the composition of any one of the preceding claims in an amount effective to induce in the subject a neutralizing antibody response against SARS-CoV-2.

Other aspects of the present disclosure provide a method comprising administering to a subject the mRNA or the composition of any one of the preceding claims in an amount effective to induce in the subject and a T cell immune response against SARS-CoV-2.

Some aspects of the present disclosure provide a messenger ribonucleic acid (mRNA) comprising an open reading frame (ORF) that encodes a coronavirus antigen capable of inducing an immune response, such as a neutralizing antibody response, to a SARS-CoV-2, wherein the antigen comprises a protein fragment or a functional protein domain of a SARS-CoV-2, optionally wherein the RNA is formulated in a lipid nanoparticle.

In some embodiments, the antigen is a functional protein domain.

In some embodiments, the protein domain is an N-terminal domain (NTD) of a SARS-CoV-2 Spike protein.

In some embodiments, the NTD is linked to a transmembrane domain, optionally an influenza hemagglutinin transmembrane domain.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 47, optionally wherein the antigen comprises the amino acid sequence of SEQ ID NO: 47.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 46, optionally wherein the open reading frame comprises the nucleotide sequence of SEQ ID NO: 46.

In some embodiments, the protein domain is a receptor binding domain (RBD) of a SARS-CoV-2 Spike protein.

In some embodiments, the RBD is soluble.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 62, optionally wherein the antigen comprises the amino acid sequence of SEQ ID NO: 62.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 61, optionally wherein the open reading frame comprises the nucleotide sequence of SEQ ID NO: 61.

In some embodiments, the RBD is linked to a transmembrane domain, optionally an influenza hemagglutinin transmembrane domain.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 77, optionally wherein the antigen comprises the amino acid sequence of SEQ ID NO: 77.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 76, optionally wherein the open reading frame comprises the nucleotide sequence of SEQ ID NOs: 76.

In some embodiments, the NTD is linked to an RBD of a SARS-CoV-2 Spike protein to form an NTD-RBD fusion protein.

In some embodiments, the NTD-RBD fusion is linked to a transmembrane domain (TM), optionally an influenza hemagglutinin transmembrane domain, to form an NTD-RBD-TM protein.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 92, optionally wherein the antigen comprises the amino acid sequence of SEQ ID NO: 92.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 91, optionally wherein the open reading frame comprises the nucleotide sequence of SEQ ID NO: 91.

In some embodiments, the NTD-RBD fusion comprises a C-terminal truncation.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 107, optionally wherein the antigen comprises the amino acid sequence of SEQ ID NO: 107.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 106, optionally wherein the open reading frame comprises the nucleotide sequence of SEQ ID NO: 106.

In some embodiments, the NTD and/or RBD includes an extended region.

In some embodiments, the antigen comprises an amino acid sequence having at least 80%, at least 85%, at least 90%, at least 95%, at least 96%, at least 97%, at least 98%, or at least 99% identity to the amino acid sequence of any one of SEQ ID NOs: 59, 86, 89, 116, 119, or 122, optionally wherein the antigen comprises the amino acid sequence of any one of SEQ ID NOs: 59, 86, 89, 116, 119, or 122.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of any one of SEQ ID NOs: 58, 85, 88, 115, 118, or 121, optionally wherein the open reading frame comprises the nucleotide sequence of any one of SEQ ID NOs: 58, 85, 88, 115, 118, or 121.

In some embodiments, the protein domain is an S1 subunit domain of a SARS-CoV-2 Spike protein.

In some embodiments, the S1 subunit is soluble.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 5, optionally wherein the antigen comprises the amino acid sequence of SEQ ID NO: 5.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 3, optionally wherein the open reading frame comprises the nucleotide sequence of SEQ ID NO: 3.

In some embodiments, the S1 subunit is linked to a transmembrane domain, optionally an influenza hemagglutinin transmembrane domain.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 17, optionally wherein the antigen comprises the amino acid sequence of SEQ ID NO: 17.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 16, optionally wherein the open reading frame comprises the nucleotide sequence of SEQ ID NO: 16.

In some embodiments, the S1 subunit has been modified to remove an RBD or a portion of an RBD of S protein.

In some embodiments, the antigen comprises an amino acid sequence having at least 80%, at least 85%, at least 90%, at least 95%, at least 96%, at least 97%, at least 98%, or at least 99% identity to the amino acid sequence of any one of SEQ ID NOs: 20, 23, 26, 29, 32 or 35, optionally wherein the antigen comprises the amino acid sequence of any one of SEQ ID NOs: 20, 23, 26, 29, 32, or 35.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of any one of SEQ ID NOs: 19, 22, 25, 28, 41, or 34, optionally wherein the open reading frame comprises the nucleotide sequence of any one of SEQ ID NOs: 19, 22, 25, 28, 31, or 34.

In some embodiments, the S1 subunit is linked to an S2 subunit of an S protein.

In some embodiments, the S2 subunit is from a SARS-CoV-2 S protein.

In some embodiments, the S1 subunit is from an HKU1 S protein.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 38, optionally wherein the antigen comprises the amino acid sequence of SEQ ID NO: 38.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 37, optionally wherein the open reading frame comprises the nucleotide sequence of SEQ ID NO: 37.

In some embodiments, the S1 subunit is from an OC43 S protein.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 41, optionally wherein the antigen comprises the amino acid sequence of SEQ ID NO: 41.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 40, optionally wherein the open reading frame comprises the nucleotide sequence of SEQ ID NO: 40.

In some embodiments, the antigen further comprises a scaffold domain, optionally selected from ferritin, lumazine synthetase and a foldon.

In some embodiments, the scaffold domain is ferritin.

In some embodiments, the antigen comprises an amino acid sequence having at least 80%, at least 85%, at least 90%, at least 95%, at least 96%, at least 97%, at least 98%, or at least 99% identity to the amino acid sequence of SEQ ID NO: 8 or 65, optionally wherein the antigen comprises the amino acid sequence of SEQ ID NO: 8 or 65.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of SEQ ID NO: 7 or 64, optionally wherein the open reading frame comprises the nucleotide sequence of SEQ ID NO: 7 or 64.

In some embodiments, the scaffold domain is lumazine synthetase.

In some embodiments, the antigen comprises an amino acid sequence having at least 80%, at least 85%, at least 90%, at least 95%, at least 96%, at least 97%, at least 98%, or at least 99% identity to the amino acid sequence of any one of SEQ ID NOs: 11, 14, 68, or 71, optionally wherein the antigen comprises the amino acid sequence of any one of SEQ ID NOs: 11, 14, 68, or 71.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of any one of SEQ ID NOs: 10, 13, 67, or 70, optionally wherein the open reading frame comprises the nucleotide sequence of any one of SEQ ID NOs: 10, 13, 67, or 70.

In some embodiments, the scaffold domain is a foldon.

In some embodiments, the antigen comprises an amino acid sequence having at least 80%, at least 85%, at least 90%, at least 95%, at least 96%, at least 97%, at least 98%, or at least 99% identity to the amino acid sequence of any one of SEQ ID NOs: 44, 50, 74, 80, 83, 101, 104 or 113, optionally wherein the antigen comprises the amino acid sequence of any one of SEQ ID NOs: 44, 50, 74, 80, 83, 101, 104 or 113.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of any one of SEQ ID NOs: 43, 49, 73, 79, 82, 100, 103, or 112, optionally wherein the open reading frame comprises the nucleotide sequence of any one of SEQ ID NOs: 43, 49, 73, 79, 82, 100, 103, or 112.

In some embodiments, the antigen further comprises a trafficking signal, optionally selected from macrophage markers, optionally CD86, CD11B and/or VSVGct.

In some embodiments, the antigen comprises an amino acid sequence having at least 80%, at least 85%, at least 90%, at least 95%, at least 96%, at least 97%, at least 98%, or at least 99% identity to the amino acid sequence of any one of SEQ ID NOs: 95, 98, or 110, optionally wherein the antigen comprises the amino acid sequence of any one of SEQ ID NOs: 95, 98, or 110.

In some embodiments, the open reading frame comprises a nucleotide sequence having at least 70%, least 75%, at least 80%, at least 90%, at least 95%, at least 96%, at least 97%, at least 98%, or at least 99% identity to the nucleotide sequence of any one of SEQ ID NOs: 94, 97, or 109, optionally wherein the open reading frame comprises the nucleotide sequence of any one of SEQ ID NOs: 94, 97, or 109.

In some embodiments, the mRNA is formulated in a lipid nanoparticle.

In some embodiments, the lipid nanoparticle comprises a cationic lipid, optionally an ionizable amino lipid, a neutral lipid, a sterol, and/or a polyethylene glycol (PEG)-modified lipid. An ionizable amino lipid is used interchangeably herein with ionizable lipid and ionizable cationic lipid to refer to an ionizable lipid. In some embodiments, the lipid nanoparticle comprises 40-50 mol % ionizable amino lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %. In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 5-15 mol % helper lipid, optionally 10-12 mol %, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol %. In some embodiments, the lipid nanoparticle comprises 1-5% PEG lipid, optionally 1-3 mol %, for example 1.5 to 2.5 mol %, 1-2 mol %, 2-3 mol %, 3-4 mol %, or 4-5 mol %.

In some embodiments, the ionizable amino lipid is heptadecan-9-yl 8 ((2 hydroxyethyl)(6 oxo 6-(undecyloxy)hexyl)amino)octanoate (Compound 1), the neutral lipid is 1,2 distearoyl-sn-glycero-3-phosphocholine (DSPC), the sterol is cholesterol, and/or the PEG-modified lipid is 1,2 dimyristoyl-sn-glycerol, methoxypolyethyleneglycol (PEG2000 DMG).

In some embodiments, the lipid nanoparticle comprises 20-60 mol % ionizable amino lipid, 5-25 mol % neutral lipid, 25-55 mol % sterol, and 0.5-15 mol % PEG-modified lipid.

In some embodiments, the lipid nanoparticle comprises: 47 mol % ionizable amino lipid; 11.5 mol % neutral lipid; 38.5 mol % sterol; and 3.0 mol % PEG-modified lipid; 48 mol % ionizable amino lipid; 11 mol % neutral lipid; 38.5 mol % sterol; and 2.5 mol % PEG-modified lipid; 49 mol % ionizable amino lipid; 10.5 mol % neutral lipid; 38.5 mol % sterol; and 2.0 mol % PEG-modified lipid; 50 mol % ionizable amino lipid; 10 mol % neutral lipid; 38.5 mol % sterol; and 1.5 mol % PEG-modified lipid; or 51 mol % ionizable amino lipid; 9.5 mol % neutral lipid; 38.5 mol % sterol; and 1.0 mol % PEG-modified lipid.

In some embodiments, the lipid nanoparticle comprises: 47 mol % Compound 1; 11.5 mol % DSPC; 38.5 mol % cholesterol; and 3.0 mol % PEG2000 DMG; 48 mol % Compound 1; 11 mol % DSPC; 38.5 mol % cholesterol; and 2.5 mol % PEG2000 DMG; 49 mol % Compound 1; 10.5 mol % DSPC; 38.5 mol % cholesterol; and 2.0 mol % PEG2000 DMG; 50 mol % Compound 1; 10 mol % DSPC; 38.5 mol % cholesterol; and 1.5 mol % PEG2000 DMG; or 51 mol % Compound 1; 9.5 mol % DSPC; 38.5 mol % cholesterol; and 1.0 mol % PEG2000 DMG.

The entire contents of International Application No. PCT/US2016/058327 (Publication No. WO2017/070626) and International Application No. PCT/US2018/022777 (Publication No. WO2018/170347) are incorporated herein by reference.

### SARS-CoV-2

The genome of SARS-CoV-2 is a single-stranded positive-sense RNA (+ssRNA) with the size of 29.8-30 kb encoding about 9860 amino acids (Chan et al. 2000, supra; Kim et al. 2020 Cell, May 14; 181(4):914-921.e10.). SARS-CoV-2 is a polycistronic mRNA with 5′-cap and 3′-poly-A tail. The SARS-CoV-2 genome is organized into specific genes encoding structural proteins and nonstructural proteins (Nsps). The order of the structural proteins in the genome is 5′-replicase (open reading frame (ORF)1/ab)-structural proteins [Spike (S)-Envelope (E)-Membrane (M)-Nucleocapsid (N)]-3′. The genome of coronaviruses includes a variable number of open reading frames that encode accessory proteins, nonstructural proteins, and structural proteins (Song et al. 2019 Viruses; 11(1):p. 59). Most of the antigenic peptides are located in the structural proteins (Cui et al. 2019 Nat. Rev. Microbiol. 17(3):181-192). Spike surface glycoprotein (S), a small envelope protein (E), matrix protein (M), and nucleocapsid protein (N) are four main structural proteins. Since S-protein contributes to cell tropism it is capable of inducing neutralizing antibodies (NAb) and protective immunity, it can be considered one of the most important targets in coronavirus vaccine development among all other structural proteins. Moreover, amino acid sequence analysis has shown that S-protein contains conserved regions among the coronaviruses, which may be the basis for universal vaccine development.

### Antigens

The compositions of the invention, e.g., vaccine compositions, feature nucleic acids, in particular, mRNAs, designed to encode an antigen of interest, e.g., an antigen derived from a betacoronavirus structural protein, in particular, antigens derived from SARS-CoV-2 Spike protein. The compositions of the invention, e.g., vaccine compositions, do not comprise antigens per se, but rather comprise nucleic acids, in particular, mRNA(s) that encode antigens or antigenic sequences once delivered to a cell, tissue or subject. Delivery of nucleic acid molecules, in particular mRNA(s) is achieved by formulating said nucleic acid molecules in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs. The term “antigen” as used herein refers to a substance such as a protein (e.g., glycoprotein), polypeptide, peptide, or the like, which elicits an immune response, e.g., elicits an immune response when present in a subject (for example, when present in a human or mammalian subject). The instant invention is based at least in part on the understanding that mRNA-encoded antigens, when expressed from mRNA administered to a cell or subject, can cause the immune system to produce an immune response to the expressed antigen, for example can trigger the production of antibodies against the expresses antigen, e.g., binding and/or neutralizing antibodies, can trigger B and or T cell responses specific to the expressed antigen, and ultimately can cause protective or prophylactic response against subsequent encounter with the antigen or with a pathogen with which the antigen is associated. Preferred mRNA-encoded antigens are “viral antigens”. As used herein, the term “viral antigen” refers to an antigen derived from a virus, for example from a pathogenic virus. The term antigen as used herein can refer to a full-length protein, for example, a full-length viral protein, or can refer to a fragment (e.g., a polypeptide or peptide fragment), subunit or domain of a protein, e.g., a viral protein subunit or domain.

Many proteins have a quaternary or three-dimensional structure, which consists of more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

In structural and molecular biology, identical, homologous or similar subunits or domains can help to classify newly identified or novel proteins, as was done immediately upon publication of the SARS-CoV-2 viral genomic sequence.

As used herein, the term antigen is distinct from the term “epitope” which is a substructure of an antigen, e.g., a polypeptide or carbohydrate structure, which may be recognized by an antigen binding site but is insufficient to induce an immune response. The art describes protein antigens that are delivered to subjects or immune cells in isolated form, e.g., isolated protein, polypeptide or peptide antigens, however, the design, testing, validation, and production of protein antigens can be costly and time-consuming, especially when producing proteins at large scale. By contrast, mRNA technology is amenable to rapid design and testing of mRNA constructs encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the invention are expressed by the cells of the subject, e.g., are expressed by the human body, and thus the subject, e.g., the human body, serves as the “factory” to produce the antigens which, in turn, elicits the desired immune response.

In preferred aspects, antigens are proteins capable of inducing an immune response (e.g., causing an immune system to produce antibodies against the antigens). Herein, use of the term “antigen” encompasses immunogenic proteins, as well as polypeptides or peptides derived from immunogenic proteins, for example immunogenic fragments (an immunogenic fragment that induces (or is capable of inducing) an immune response to an antigen, unless otherwise stated. It should be understood that the term “protein” encompasses polypeptides and peptides and the term “antigen” encompasses antigenic fragments. Other molecules may be antigenic such as bacterial polysaccharides or combinations of protein and polysaccharide structures, but for the viral vaccines included herein, viral proteins, fragments of viral proteins and designed and or mutated proteins derived from the betacoronavirus SARS-CoV-2 are the antigens featured herein.

### Nucleic Acids/mRNA

The vaccine technology described herein features nucleic acids, particularly messenger RNA (mRNA) designed to encode an antigen of interest, e.g., a betacoronavirus spike protein antigen, subunit, domain or fragments (e.g., antigenic fragments) thereof. The nucleic acids, for example mRNAs, of the invention are preferably formulated in appropriate carriers or delivery vehicles (e.g., lipid nanoparticles), such that the nucleic acids, e.g., mRNAs are suitable for use in vivo. When appropriately formulated, nucleic acids, e.g., mRNAs, are capable of being delivered to cells and/or tissues within a subject, e.g., a human subject, to effectuate translation of protein encoded by these nucleic acids.

Nucleic acid molecules are macromolecules comprised of linked nucleotides that carry that carry genetic information and by directing the process of protein synthesis, direct most if not all cellular functions. Nucleic acids comprise a polymer of nucleotides (nucleotide monomers). Thus, nucleic acids are also referred to as polynucleotides (also referred to as polynucleotide chains). The two main classes of nucleic acids are deoxyribonucleic acid (DNA) and ribonucleic acid (RNA). DNA constitutes the genetic material in all free-living organisms and most viruses. RNA is the genetic material of certain viruses, but it is also found in all living cells, where it plays an important role cellular processes, most notably the making of proteins.

Nucleosides are the structural subunit of nucleic acids such as DNA and RNA. A nucleoside is composed of a nitrogenous base (a nucleobase), usually either a pyrimidine (cytosine, thymine or uracil) or a purine (adenine or guanine), covalently attached to a five-carbon carbohydrate ribose or “sugar” which is either ribose or deoxyribose. Nucleotides consist of a nitrogenous base, a sugar (ribose or deoxyribose) and one to three phosphate groups. In essence, a nucleotide is simply a nucleoside with an additional phosphate group or groups.

The nucleic acid molecules, DNA and RNA, are composed of nucleotides that are linked to one another in a chain by chemical bonds, called ester bonds, between the sugar base of one nucleotide and the phosphate group of the adjacent nucleotide. The sugar is the 3′ end, and the phosphate is the 5′ end of each nucleotide. The phosphate group attached to the 5′ carbon of the sugar on one nucleotide forms an ester bond with the free hydroxyl on the 3′ carbon of the next nucleotide. These bonds are called phosphodiester bonds, and the sugar-phosphate backbone is described as extending, or growing, in the 5′ to 3′ direction when the molecule is synthesized.

The nucleobase portion of nucleic acids features purine bases, adenine (A) and guanine (G), and pyrimidine bases, cytosine (C), thymine (T) in DNA, and uracil (U) in RNA. The sugar portion of nucleic acids features deoxyribose in DNA, ribose in RNA. The five nucleosides are commonly abbreviated to their one-letter codes A, G, C, T and U, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G whereas in DNA they would be represented as dA, dC and dG.

The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

Nucleic acids may be or may include, for example, deoxyribonucleic acids (DNAs), ribonucleic acids (RNAs), e.g. mRNAs, threose nucleic acids (TNAs), glycol nucleic acids (GNAs), peptide nucleic acids (PNAs), locked nucleic acids (LNAs, including LNA having a (3-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Featured in the instant invention are messenger RNAs (mRNAs), particularly mRNAs designed to encode an antigen of interest, e.g., a betacoronavirus spike protein antigen, subunit, domain or fragments (e.g., antigenic fragments) thereof. Messenger RNA (mRNA), a subtype of RNA, is a single-stranded molecule of RNA that corresponds to the genetic sequence of a gene. mRNA is created during the process of transcription wherein a single strand of DNA is decoded by RNA polymerase, and mRNA is synthesized, i.e., transcribed. mRNA is read by a ribosome in the process of synthesizing a protein, i.e., translation. Accordingly, messenger RNA (mRNA) is an RNA that encodes a (at least one) protein (a naturally-occurring, non-naturally-occurring, or modified polymer of amino acids) and can be translated to produce the encoded protein in vitro, in vivo, in situ, or ex vivo.

The compositions of the present disclosure comprise a (at least one) mRNA having an open reading frame (ORF) encoding a coronavirus antigen. In some embodiments, the mRNA further comprises a 5′ UTR, 3′ UTR, a poly(A) tail and/or a 5′ cap or cap analog. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an mRNA of the present disclosure. It should also be understood that the mRNAs if the invention, e.g., mRNAs featured in the betacoronavirus vaccines of the present disclosure, may include any 5′ untranslated region (UTR) and/or any 3′ UTR. Exemplary UTR sequences are provided in the Sequence Listing (e.g., SEQ ID NOs: 2, 4, 131, and 132); however, other UTR sequences may be used or exchanged for any of the UTR sequences described herein. UTRs may also be omitted from the mRNAs provided herein.

In some embodiments, a composition comprises an mRNA that comprises a nucleotide sequence having at least 70%, at least 75%, at least 80%, at least 85%, at least 90%, at least 95%, at least 96%, at least 97%, at least 98%, at least 99%, or 100% identity to the nucleotide sequence of any one of SEQ ID NOs: 45, 75, or 90. In some embodiments, a composition comprises an mRNA that comprises a nucleotide sequence having at least 70%, at least 75%, at least 80%, at least 85%, at least 90%, at least 95%, at least 96%, at least 97%, at least 98%, at least 99%, or 100% identity to the nucleotide sequence of any one of the sequences in Tables 1-15.

In some embodiments, a composition comprises an mRNA that comprises an ORF having at least 70%, at least 75%, at least 80%, at least 85%, at least 90%, at least 95%, at least 96%, at least 97%, at least 98%, at least 99%, or 100% identity to the nucleotide sequence of any one of SEQ ID NOs: 46, 76, or 91. In some embodiments, a composition comprises an mRNA that comprises an ORF having at least 70%, at least 75%, at least 80%, at least 85%, at least 90%, at least 95%, at least 96%, at least 97%, at least 98%, at least 99%, or 100% identity to the nucleotide sequence of any one of the sequences in Table 1-15.

Exemplary sequences of the coronavirus antigens and the mRNA encoding the coronavirus antigens of the compositions of the present disclosure are provided in Tables 1-15.

It should be understood that any one of the antigens encoded by the mRNA described herein may or may not comprise a signal sequence.

### Encoded Coronavirus Spike (S) Protein Antigens

The envelope spike (S) proteins of known betacoronaviruses determine the virus host tropism and entry into host cells. Coronavirus spike (S) protein is a choice antigen for the vaccine design as it can induce neutralizing antibodies and protective immunity. S protein is critical for SARS-CoV-2 infection. The organization of the S protein is similar among betacoronaviruses, such as SARS-CoV-2, SARS-CoV, MERS-CoV, HKU1-CoV, MHV-CoV and NL63-CoV.

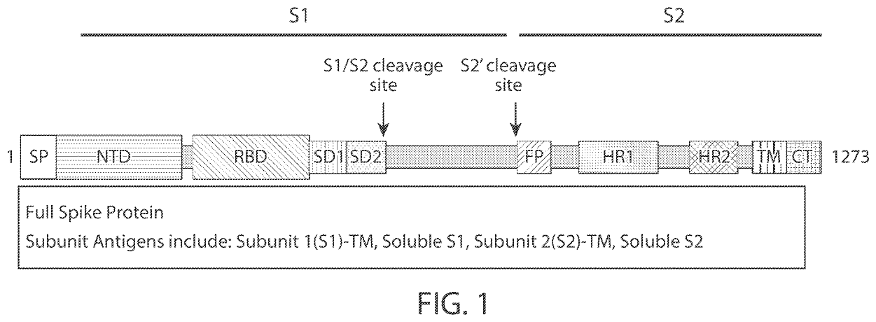

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids and comprise the structure (i.e., the domain composition and organization) as set forth in FIG. 1. SARS-CoV-2 spike (S) protein is a choice antigen for the vaccine design as it can induce neutralizing antibodies and protective immunity. mRNAs of the invention are designed to produce SARS-CoV-2 Spike proteins (i.e., encode Spike proteins such that Spike protein is expressed when the mRNA is delivered to a cell or tissue, for example a cell or tissue in a subject), as well as antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. In exemplary embodiments, a Spike protein, e.g., an encoded Spike protein antigen, has the amino acid sequence set forth in any one of the sequences of Tables 1-15 (e.g., derived from the amino acid sequence set forth as SEQ ID NO: 125). In other embodiments, a Spike protein, e.g., an encoded Spike protein antigen, has no greater than 100, no greater than 90, no greater than 80, no greater than 70, no greater than 60, no greater than 50, no greater than 40, no greater than 30, no greater than 20, no greater than 10, or no greater than 5 amino acid substitutions and/or deletions as compared to (when aligned with) a Spike protein having the amino acid sequence as set forth in any one of the sequences of Tables 1-15 (e.g., derived from the amino acid sequence set forth as SEQ ID NO: 125). Where minor variations are made in encoded Spike protein sequences, the variant preferably has the same activity as the reference Spike protein sequence and/or has the same immune specificity as the reference Spike protein, as determined for example, in immunoassays (e.g., enzyme-linked immunosorbent assays (ELISA assays).

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, specifically angiotensin-converting enzyme 2 (ACE2) receptors, whereas the S2 subunit, which is the most conserved component of the S protein, will be responsible for fusing the envelope of the virus with the host cell membrane. (See e.g., Shang et al., PLoS Pathog. 2020 March; 16(3):e1008392.). Each monomer of trimeric S protein trimer contains the two subunits, S1 and S2, mediating attachment and membrane fusion, respectively. See, e.g., FIG. 1. As part of the infection process in vivo, the two subunits are separated from each other by an enzymatic cleavage process. S protein is first cleaved by furin-mediated cleavage at the S1/S2 site in infected cells, In vivo, a subsequent serine protease-mediated cleavage event occurs at the S2′ site within S1. In SARS-CoV2, the S1/S2 cleavage site is at amino acids 676-TQTNSPRRAR/SVA-688 (referencing SEQ ID NO: 127). The S2′ cleavage site is at amino acids 811-KPSKR/SFI-818 (referencing SEQ ID NO: 126).

As used herein, for example in the context of designing SARS-CoV-2 S protein antigens encoded by the nucleic acids, e.g., mRNAs, of the invention, the term “S1 subunit” (e.g., S1 subunit antigen) refers to the N-terminal subunit of the Spike protein beginning at the S protein N-terminus and ending at the S1/S2 cleavage site whereas the term “S2 subunit” (e.g., S2 subunit antigen) refers to the C-terminal subunit of the Spike protein beginning at the S1/S2 cleavage site and ending at the C-terminus of the Spike protein. As described supra, the skilled artisan will understand that, while an essentially full length or complete Spike protein S1 or S2 subunit may be necessary for receptor binding or membrane fusion, respectively, a certain amount of variation in S1 or S2 structure and/or sequence is tolerated when seeking primarily to elicit an immune response against Spike protein subunits. For example, minor truncation, e.g., of one to a few, possibly up to 4, 5, 6, 7, 8, 9 or 10 amino acids from the N- or C-terminus of the encoded subunit, e.g., encoded S1 or S2 protein antigens, may be tolerated without changing the antigenic properties of the protein. Likewise, variation (e.g., conservative substitution) of one to a few, possibly up to 4, 5, 6, 7, 8, 9 or 10 amino acids (or more) of the encoded Spike protein subunits, e.g., encoded S1 or S2 protein antigen, may be tolerated without changing the antigenic properties of the protein(s). In exemplary embodiments, a Spike protein, e.g., an encoded Spike protein antigen, has the amino acid sequence set forth in any one of the sequences of Tables 1-15 (e.g., derived from the amino acid sequence set forth as SEQ ID NO: 125). In other embodiments, a Spike protein subunit, e.g., an encoded S1 or S2 protein antigen, has no greater than 50, no greater than 40, no greater than 30, no greater than 20, no greater than 10, or no greater than 5 amino acid substitutions and/or deletions as compared to (when aligned with) a Spike protein S1 subunit comprising or consisting of amino acids 1-685 or a Spike protein S2 subunit comprising or consisting of amino acids 686-1273 of the Spike protein having the amino acid sequence as set forth as SEQ ID NO: 125. Where minor variations are made in encoded Spike protein subunit sequences, the variant preferably has the same activity as the reference Spike protein subunit sequence and/or has the same immune specificity as the reference Spike protein subunit, as determined for example, in immunoassays (e.g., enzyme-linked immunosorbent assays (ELISA assays).

The S1 and S2 subunits of the SARS-CoV-2 Spike protein further include domains readily discernable by structure and function, which in turn can be featured in designing antigens to be encoded by the nucleic acid vaccines, in particular, mRNA vaccines of the invention. Within the S1 subunit, domains include the N-terminal domain (NTD) and the receptor-binding domain (RBD), said RBD domain further including a receptor-binding motif (RBM). The wild type S1 subunit also includes a signal peptide (SD), N-terminal to the NTD domain and a first subdomain (SD1) and second subdomain (SD2). Within the S2 subunit, domains include fusion peptide (FP), heptad repeat 1 (HR1), heptad repeat 2 (HR2), transmembrane domain (TM), and cytoplasm domain, also known as cytoplasmic tail (CT) (Lu R. et al., supra; Wan et al., J. Virol. March 2020, 94 (7) e00127-20). The HR1 and HR2 domains can be referred to as the “fusion core region” of SARS-CoV-2 (Xia et al., 2020 Cell Mol Immunol. January; 17(1):1-12.). FIG. 1 depicts the domain architecture in the SARS-CoV-2 Spike protein. The S1 subunit includes an N terminal domain (NTD), a linker region, a receptor binding domain (RBD), a first subdomain (SD1), and a second subdomain (SD2). An S1 subunit may be modified to add a C-terminal transmembrane domain (TM) or it may be soluble. The S2 subunit includes, inter alia, a first heptad repeat (HR1), a second heptad repeat (HR2), a transmembrane domain (TM), and a cytoplasmic tail. A soluble S2 subunit may be generated without a TM domain.

The NTD and RBD of S1 are good antigens for the vaccine design approach of the invention as these domains have been shown to be the targets of neutralizing antibodies in betacoronavirus-infected individuals. As used herein, for example, in the context of an antigen design (said antigen encoded by an mRNA of the invention and to be expressed, for example, from and mRNA vaccine of the invention), the term “N-terminal domain” or “NTD” refers to a domain within the SARS-CoV-2 S1 subunit comprising approximately 290 amino acids in length, having identity to amino acids 1-290 of the S1 subunit of the Spike protein having the amino acid sequence set forth as SEQ ID NO: 125. As used herein, for example, in the context of an antigen design (said antigen encoded by an mRNA of the invention and to be expressed, for example, from and mRNA vaccine of the invention), the term “receptor binding domain” or “RBD” refers to a domain within the S1 subunit of SARS-CoV-2 comprising approximately 175-225 amino acids in length, having identity to amino acids 316-517 of the S1 subunit of the Spike protein having the amino acid sequence set forth as SEQ ID NO: 125. As used herein, the term “receptor binding motif” refers to the portion of the RBD that directly contacts the ACE2 receptor. Expressed RBDs are predicted to specifically bind to angiotensin-converting enzyme 2 (ACE2) as its receptor and/or specifically react with RBD-binding and/or neutralizing antibodies, e.g., CR3022.

The compositions provided herein include mRNA that may encode any one or more full-length or partial (truncated or other deletion of sequence) S protein subunit (e.g., S1 or S2 subunit), one or more domain or combination of domains of an S protein subunit (e.g., NTD, RBD, or NTD-RBD fusions, with or without an SD1 and/or SD2), or chimeras of full-length or partial and S2 protein subunits. Other S protein subunit and/or domain configurations are contemplated herein.

### Encoded Subunit Antigens

Some aspects of the present disclosure provide compositions comprising an mRNA that encodes a (at least one) subunit of a SARS-CoV-2 S protein. In some embodiments, the mRNA encodes an S1 subunit (e.g., full length or partial). In other embodiments, the mRNA encodes an S2 subunit (e.g., full length or partial). In yet other embodiments, the mRNA encodes a chimeric S1-S2 protein, wherein one of the subunits is from a SARS-CoV-2 S protein, and the other subunit is from another organism, e.g., a virus, such influenza virus. The SARS-CoV-2 subunits (S1 and/or S2) encoded by the mRNA of the present disclosure may be soluble or membrane bound (e.g., linked to a transmembrane domain).

### Soluble Subunit Antigens

A soluble protein is present in the cytoplasm of a cell or is secreted from a cell (e.g., not membrane bound). Soluble antigens secreted by cells may be opsonized by complement and captured by follicular dendritic cells in lymph nodes, where they may be recognized by B cells specific to epitopes present on the expressed protein. The expression of subunit antigens further allows focusing of the immune response to specific subunits and with minimal stimulation of memory B and T cells specific to other domains of the antigen that are shared with other related viruses. Without being bound by theory, it is thought that presentation of the SARS-CoV-2 S1 subunit, including the NTD, the RBD, and, in some instances, the intervening polypeptides of the SARS-CoV-2 S1 subunit, in soluble form, generates an S1 subunit-specific immune response. Thus, in some embodiments, an mRNA provided herein encodes a soluble SARS-CoV-2 S subunit antigen and/or a soluble SARS-CoV-2 S2 subunit antigen. A non-limiting example of a soluble SARS-CoV-2 S1 subunit antigen and the mRNA encoding it is provided in Tables 1A and 1B below. Other examples of soluble SARS-CoV-2 subunit antigens are provided herein.

TABLE 1A
Soluble Subunit Antigen

SEQ ID NO:

| Name | mRNA ORF | Protein |
|---|---|---|
| SARS-CoV-2 Soluble S1 Subunit | 3 | 5 |

TABLE 1B
Soluble Subunit Antigen
SARS-CoV-2 Soluble S1 Subunit

| | |
|---|---|
| SEQ ID NO: 1 consists of from 5′ end to 3′ end: | 1 |
| 5′ UTR SEQ ID NO: 2, | |
| mRNA ORF SEQ ID NO: 3 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 3 |
| Construct | UGAACCUGACCACCCGGACCCAGCUGCCACCAGCCUACACCAACAG | |
| (excluding | CUUCACCCGGGGCGUCUACUACCCCGACAAGGUGUUCCGGAGCAGC | |
| the stop | GUCCUGCACAGCACCCAGGACCUGUUCCUGCCCUUCUUCAGCAACG | |
| codon) | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCA | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding | MFVFLVLLPLVSSQCVNLITTRTQLPPAYTNSFTRGVYYPDKVERSS | 5 |
| amino | VLHSTQDLFLPFFSNVTWFHAIHVSGINGTKRFDNPVLPENDGVYF | |
| acid sequence | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKELPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNS | |
| PolyA tail | 100 nt | |

### Membrane Bound Subunit Antigens

A membrane bound protein is anchored in a cell membrane with a transmembrane domain and is therefore not soluble. Without being bound by theory, it is thought that antigen presenting cells will carry the embedded antigen to the draining lymph nodes to generate a strong immune response. The germinal center reaction that occurs in the draining lymph node involves prolonged contact between CD4⁺ T_{FH} cells and B cells, allowing co-stimulation and local cytokine signals such as IL-4 and JL-21 that favor replication of B cells specific to the presented antigen and class switching to the production of IgG1, each of which may promote the generation of long-lived plasma cells and memory B cells. Thus, in some embodiments, an mRNA encodes a membrane bound SARS-CoV-2 S1 subunit antigen and/or a membrane bound SARS-CoV-2 S2 subunit antigen. In some embodiments, a membrane bound antigen (e.g., S1 subunit, S2 subunit, NTD, RBD, or any combination thereof) is linked to a transmembrane domain, e.g., a naturally occurring transmembrane domain or a heterologous transmembrane domain (derived from a heterologous protein), which is responsible for anchoring the protein in the cell membrane. A non-limiting example of a membrane bound SARS-CoV-2 S1 subunit antigen and a SARS-CoV-2 S2 subunit antigen and the mRNA encoding them are provided in Tables 2A and 2B below. Other membrane bound SARS-CoV-2 S1 subunit antigens are contemplated herein.

TABLE 2A
Membrane Bound Subunit Antigen

SEQ ID NO:

| Name | mRNA ORF | Protein |
|---|---|---|
| SARS-CoV-2 S1 Subunit Linked to | 16 | 17 |
| Transmembrane Domain (S1-666-TM) | | |
| SARS-CoV-2 S2 Subunit Linked to | 145 | 146 |
| Transmembrane Domain (S2-TM) | | |

TABLE 2B
Membrane Bound Subunit Antigen
SARS-CoV-2 S1 Subunit Linked to Transmembrane Domain (S1-666-TM)

| SEQ ID NO: 15 consists of from 5′ end to 3′ end: | | 15 |
|---|---|---|
| 5′ UTR SEQ ID NO: 2, | | |
| mRNA ORF SEQ ID NO: 16 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 16 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCAUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 17 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNSGGGSILAIYS | |
| | TVASSLVLLVSLGAISF | |
| PolyA tail | 100 nt | |

SARS-CoV-2 S2 Subunit comprising a Transmembrane Domain (S2-TM)

| SEQ ID NO: 147 consists of from 5′ end to 3′ end: | | 147 |
|---|---|---|
| 5′ UTR SEQ ID NO: 2, | | |
| mRNA ORF SEQ ID NO: 145 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 145 |
| Construct | UGCUGGUGAACAGCCAGGGCGCCGAGAACAGCGUGGCCUACAGCAA | |
| (excluding the stop | CAACAGCAUCGCCAUCCCCACCAACUUCACCAUCAGCGUGACCACC | |
| codon) | GAGAUUCUGCCCGUGAGCAUGACCAAGACCAGCGUGGACUGCACCA | |
| | UGUACAUCUGCGGCGACAGCACCGAGUGCAGCAACCUGCUGCUGCA | |
| | GUACGGCAGCUUCUGCACCCAGCUGAACCGGGCCCUGACCGGCAUC | |
| | GCCGUGGAGCAGGACAAGAACACCCAGGAGGUGUUCGCCCAGGUGA | |
| | AGCAGAUCUACAAGACCCCUCCCAUCAAGGACUUCGGCGGCUUCAA | |
| | CUUCAGCCAGAUCCUGCCCGACCCCAGCAAGCCCAGCAAGCGGAGC | |
| | UUCAUCGAGGACCUGCUGUUCAACAAGGUGACCCUAGCCGACGCCG | |
| | GCUUCAUCAAGCAGUACGGCGACUGCCUCGGCGACAUAGCCGCCCG | |
| | GGACCUGAUCUGCGCCCAGAAGUUCAACGGCCUGACCGUGCUGCCU | |
| | CCCCUGCUGACCGACGAGAUGAUCGCCCAGUACACCAGCGCCCUGU | |
| | UAGCCGGAACCAUCACCAGCGGCUGGACUUUCGGCGCUGGAGCCGC | |
| | UCUGCAGAUCCCCUUCGCCAUGCAGAUGGCCUACCGGUUCAACGGC | |
| | AUCGGCGUGACCCAGAACGUGCUGUACGAGAACCAGAAGCUGAUCG | |
| | CCAACCAGUUCAACAGCGCCAUCGGCAAGAUCCAGGACAGCCUGAG | |
| | CAGCACCGCUAGCGCCCUGGGCAAGCUGCAGGACGUGGUGAACCAG | |
| | AACGCCCAGGCCCUGAACACCCUGGUGAAGCAGCUGAGCAGCAACU | |
| | UCGGCGCCAUCAGCAGCGUGCUGAACGACAUCCUGAGCCGGCUGGA | |
| | CCCUCCCGAGGCCGAGGUGCAGAUCGACCGGCUGAUCACUGGCCGG | |
| | CUGCAGAGCCUGCAGACCUACGUGACCCAGCAGCUGAUCCGGGCCG | |
| | CCGAGAUUCGGGCCAGCGCCAACCUGGCCGCCACCAAGAUGAGCGA | |
| | GUGCGUGCUGGGCCAGAGCAAGCGGGUGGACUUCUGCGGCAAGGGC | |
| | UACCACCUGAUGAGCUUUCCCCAGAGCGCACCCCACGGAGUGGUGU | |
| | UCCUGCACGUGACCUACGUGCCCGCCCAGGAGAAGAACUUCACCAC | |
| | CGCCCCAGCCAUCUGCCACGACGGCAAGGCCCACUUUCCCCGGGAG | |
| | GGCGUGUUCGUGAGCAACGGCACCCACUGGUUCGUGACCCAGCGGA | |
| | ACUUCUACGAGCCCCAGAUCAUCACCACCGACAACACCUUCGUGAG | |
| | CGGCAACUGCGACGUGGUGAUCGGCAUCGUGAACAACACCGUGUAC | |
| | GAUCCCCUGCAGCCCGAGCUGGACAGCUUCAAGGAGGAGCUGGACA | |
| | AGUACUUCAAGAAUCACACCAGCCCCGACGUGGACCUGGGCGACAU | |
| | CAGCGGCAUCAACGCCAGCGUGGUGAACAUCCAGAAGGAGAUCGAU | |
| | CGGCUGAACGAGGUGGCCAAGAACCUGAACGAGAGCCUGAUCGACC | |
| | UGCAGGAGCUGGGCAAGUACGAGCAGUACAUCAAGUGGCCCUGGUA | |
| | CAUCUGGCUGGGCUUCAUCGCCGGCCUGAUCGCCAUCGUGAUGGUG | |
| | ACCAUCAUGCUGUGCUGCAUGACCAGCUGCUGCAGCUGCCUGAAGG | |
| | GCUGUUGCAGCUGCGGCAGCUGCUGCAAGUUCGACGAGGACGACAG | |
| | CGAGCCCGUGCUGAAGGGCGUGAAGCUGCACUACACC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MYSMQLASCVTLTLVLLVNSQGAENSVAYSNNSIAIPTNFTISVTT | 146 |
| acid sequence | EILPVSMTKTSVDCTMYICGDSTECSNLLLQYGSFCTQLNRALTGI | |
| | AVEQDKNTQEVFAQVKQIYKTPPIKDFGGFNFSQILPDPSKPSKRS | |
| | FIEDLLENKVTLADAGFIKQYGDCLGDIAARDLICAQKFNGLTVLP | |
| | PLLTDEMIAQYTSALLAGTITSGWTFGAGAALQIPFAMQMAYRENG | |
| | IGVTQNVLYENQKLIANQFNSAIGKIQDSLSSTASALGKLQDVVNQ | |
| | NAQALNTLVKQLSSNFGAISSVLNDILSRLDPPEAEVQIDRLITGR | |
| | LQSLQTYVTQQLIRAAEIRASANLAATKMSECVLGQSKRVDFCGKG | |
| | YHLMSFPQSAPHGVVFLHVTYVPAQEKNFTTAPAICHDGKAHFPRE | |
| | GVFVSNGTHWFVTQRNFYEPQIITTDNTFVSGNCDVVIGIVNNTVY | |
| | DPLQPELDSFKEELDKYFKNHTSPDVDLGDISGINASVVNIQKEID | |
| | RLNEVAKNLNESLIDLQELGKYEQYIKWPWYIWLGFIAGLIAIVMV | |
| | TIMLCCMTSCCSCLKGCCSCGSCCKFDEDDSEPVLKGVKLHYT | |
| PolyA tail | 100 nt | |

### Subunit Antigen Truncations and RBD Deletions

In some embodiments, a composition comprises an mRNA that encodes an S1 subunit that has been modified to remove the RBD or a portion of the RBD. Truncation of the S1 subunit provides fewer epitopes for the immune system to recognize, thereby biasing the immune response to the remaining epitopes, which may select for antibodies to specific epitopes that are important for virus neutralization. Truncation or partial deletion of the RBD may prevent the expressed protein or cells carrying it from interacting with receptor ACE2, making it more likely to reach the lymph node and stimulate a desired immune response. Furthermore, removing the RBD may prevent epitope masking by cross-reactive antibodies previously raised against related viruses, and thus focus the elicited immune response toward the desired antigen specifically. Additionally, removal of the RBD may alter the conformation of the expressed subunit, allowing B cells specific to these alternative conformational epitopes to uptake and present linear peptides to T cells, thereby indirectly enhancing the CD4⁺ T cell response to those epitopes, which are still present in the native conformation.

In some embodiments, a composition comprises an mRNA that encodes an S1 subunit that has been modified to remove the RBD or a portion of the RBD, wherein the S2 subunit contains a glycan. Glycans are attached to proteins by N-linked glycosylation via asparagine residues or O-linked glycosylation on serine or threonine residues. The presence of a glycan T shield on some components of a protein may mask peptide epitopes, thereby focusing the antibody response towards other exposed peptide epitopes. Furthermore, glycosylated proteins also elicit antibodies that recognize the coating glycans. B cells that recognize the glycan epitope will intake and present linear peptide epitopes to CD4⁺ T cells, thereby boosting the CD4 T cell response to linear epitopes found throughout the protein.

Non-limiting examples of truncated SARS-CoV-2 S subunit antigens and the mRNA encoding them are provided in Tables 3A and 3B below.

Non-limiting examples of SARS-CoV-2 S subunits having an RBD deletion and the mRNA encoding them are provided in Tables 4A and 4B below.

TABLE 3A
Subunit Antigen Truncations

| | SEQ ID NO: | |
|---|---|---|
| | mRNA | |
| Name | ORF | Protein |
| SARS-CoV-2 S1 Subunit Truncated and Linked | 19 | 20 |
| to Transmembrane Domain (S1-531-TM) | | |
| SARS-CoV-2 S1 Subunit Truncated and Linked | 22 | 23 |
| to Transmembrane Domain (S1-594-TM) | | |
| SARS-CoV-2 S1 Subunit Truncated with PolyG | 25 | 26 |
| and Linked to Transmembrane Domain | | |
| (S1-594-PolyG-TM) | | |
| SARS-CoV-2 S1 Subunit Truncated with PolyG/ | 28 | 29 |
| DS and Linked to Transmembrane Domain | | |
| (S1-594-PolyG-DS-TM) | | |
| SARS-CoV-2 S1 Subunit Truncated and Linked | 150 | 151 |
| to Transmembrane Domain (S1-666-TM) | | |

TABLE 3B
Subunit Antigen Truncations
SARS-CoV-2 S1 Subunit Truncated and Linked to Transmembrane Domain (S1-531-TM)

| SEQ ID NO: 18 consists of from 5′ end to 3′ end: 5′ UTR SEQ ID NO: 2, | | 18 |
|---|---|---|
| mRNA ORF SEQ ID NO: 19 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 19 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCUCUGGCGGAGGCAGCAU | |
| | CCUGGCCAUCUACAGCACCGUGGCCAGCAGCCUGGUGCUGCUGGUG | |
| | AGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVFRSS | 20 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTsgggsilaiystvasslvllv | |
| | slgaisf | |
| PolyA tail | 100 nt | |

SARS-CoV-2 S1 Subunit Truncated and Linked to Transmembrane Domain (S1-594-TM)

| SEQ ID NO: 21 consists of from 5′ end to 3′ end: 5′ UTR SEQ ID NO: 2, | | 21 |
|---|---|---|
| mRNA ORF SEQ ID NO: 22 and 3′ UTR SEQ ID NO: 4. | | |
| Chemistry | 1 -methylpseudouridine | |
| Cap | 7mG(5′)ppp(5′)NImpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 22 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCUCUGGCGGAGGC | |
| | AGCAUCCUGGCCAUCUACAGCACCGUGGCCAGCAGCCUGGUGCUGC | |
| | UGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 23 |
| acid sequence | VLHSTQDLFLPFFSNVTWFHAIHVSGTNGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVFNATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGsggg | |
| | silaiystvasslvllvslgaisf | |
| PolyA tail | 100 nt | |

SARS-CoV-2 S1 Subunit Truncated with PolyG and Linked to
Transmembrane Domain (S1-594-PolyG-TM)

| SEQ ID NO: 24 consists of from 5′ end to 3′ end: 5′ UTR SEQ ID NO: 2, | | 24 |
|---|---|---|
| mRNA ORF SEQ ID NO: 25 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 25 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGGCAGCGGCGGCGGCAGCGGCGGAGGCAGCGGAGGAGGCAGC | |
| | GGCGGAGGCAGUGGAGGCCAGCCCACCGAGAGCAUCGUGCGGUUCC | |
| | CCAACAUCACCAACCUGUGCCCCUUCGGCGAGGUGUUCAACGCCAC | |
| | CCGGUUCGCCAGCGUGUACGCCUGGAACCGGAAGCGGAUCAGCAAC | |
| | UGCGUGGCCGACUACAGCGUGCUGUACAACAGCGCCAGCUUCAGCA | |
| | CCUUCAAGUGCUACGGCGUGAGCCCCACCAAGCUGAACGACCUGUG | |
| | CUUCACCAACGUGUACGCCGACAGCUUCGUGAUCCGUGGCGACGAG | |
| | GUGCGGCAGAUCGCACCCGGCCAGACAGGCAAGAUCGCCGACUACA | |
| | ACUACAAGCUGCCCGACGACUUCACCGGCUGCGUGAUCGCCUGGAA | |
| | CAGCAACAACCUCGACAGCAAGGUGGGCGGCAACUACAACUACCUG | |
| | UACCGGCUGUUCCGGAAGAGCAACCUGAAGCCCUUCGAGCGGGACA | |
| | UCAGCACCGAGAUCUACCAAGCCGGCUCCACCCCUUGCAACGGCGU | |
| | GGAGGGCUUCAACUGCUACUUCCCUCUGCAGAGCUACGGCUUCCAG | |
| | CCCACCAACGGCGUGGGCUACCAGCCCUACCGGGUGGUGGUGCUGA | |
| | GCUUCGAGCUGCUGCACGCCCCAGCCACCGUGUGUGGCCCCAAGAA | |
| | GAGCACCAACCUGGUGAAGAACAAGUGCGUGAACUUCAACUUCAAC | |
| | GGCCUUACCGGCACCGGCGUGCUGACCGAGAGCAACAAGAAAUUCC | |
| | UGCCCUUUCAGCAGUUCGGCCGGGACAUCGCCGACACCACCGACGC | |
| | UGUGCGGGAUCCCCAGACCCUGGAGAUCCUGGACAUCACCCCUUGC | |
| | AGCUUCGGCGGCUCUGGCGGAGGCAGCAUCCUGGCCAUCUACAGCA | |
| | CCGUGGCCAGCAGCCUGGUGCUGCUGGUGAGCCUGGGCGCCAUCAG | |
| | CUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 26 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVGSGGGSGGGSGGGS | |
| | GGGSGGQPTESIVRFPNITNLCPFGEVENATRFASVYAWNRKRISN | |
| | CVADYSVLYNSASFSTFKCYGVSPTKLNDLCFTNVYADSFVIRGDE | |
| | VRQIAPGQTGKIADYNYKLPDDFTGCVIAWNSNNLDSKVGGNYNYL | |
| | YRLFRKSNLKPFERDISTEIYQAGSTPCNGVEGENCYFPLQSYGFQ | |
| | PTNGVGYQPYRVVVLSFELLHAPATVCGPKKSTNLVKNKCVNENEN | |
| | GLTGTGVLTESNKKFLPFQQFGRDIADTTDAVRDPQTLEILDITPC | |
| | SFGGsgggsilaiystvasslvllvslgaisf | |
| PolyA tail | 100 nt | |

SARS-CoV-2 S1 Subunit Truncated with PolyG/DS and Linked to Transmembrane
Domain (S1-594-PolyG-DS-TM)

| SEQ ID NO: 27 consists of from 5′ end to 3′ end: 5′ UTR SEQ ID NO: 2, | | 27 |
|---|---|---|
| mRNA ORF SEQ ID NO: 28 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| ORF of mRNA | GCGCCGCCACC | |
| Construct | AUGUUCGUGUUCCUGGUGCUGCUGCCCCUGGUGAGCAGCCAGUGCG | 28 |
| (excluding the stop | UGAACCUGACCACCCGGACCCAGCUGCCACCAGCCUACACCAACAG | |
| codon) | CUUCACCCGGGGCGUCUACUACCCCGACAAGGUGUGCCGGAGCAGC | |
| | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGGCAGCGGCGGCGGCAGCGGCGGAGGCAGCGGAGGAGGCAGC | |
| | GGCGGAGGCAGUGGAGGCCAGCCCACCGAGAGCAUCGUGCGGUUCC | |
| | CCAACAUCACCAACCUGUGCCCCUUCGGCGAGGUGUUCAACGCCAC | |
| | CCGGUUCGCCAGCGUGUACGCCUGGAACCGGAAGCGGAUCAGCAAC | |
| | UGCGUGGCCGACUACAGCGUGCUGUACAACAGCGCCAGCUUCAGCA | |
| | CCUUCAAGUGCUACGGCGUGAGCCCCACCAAGCUGAACGACCUGUG | |
| | CUUCACCAACGUGUACGCCGACAGCUUCGUGAUCCGUGGCGACGAG | |
| | GUGCGGCAGAUCGCACCCGGCCAGACAGGCAAGAUCGCCGACUACA | |
| | ACUACAAGCUGCCCGACGACUUCACCGGCUGCGUGAUCGCCUGGAA | |
| | CAGCAACAACCUCGACAGCAAGGUGGGCGGCAACUACAACUACCUG | |
| | UACCGGCUGUUCCGGAAGAGCAACCUGAAGCCCUUCGAGCGGGACA | |
| | UCAGCACCGAGAUCUACCAAGCCGGCUCCACCCCUUGCAACGGCGU | |
| | GGAGGGCUUCAACUGCUACUUCCCUCUGCAGAGCUACGGCUUCCAG | |
| | CCCACCAACGGCGUGGGCUACCAGCCCUACCGGGUGGUGGUGCUGA | |
| | GCUUCGAGCUGCUGCACGCCCCAGCCACCGUGUGUGGCCCCAAGAA | |
| | GAGCACCAACCUGGUGAAGAACAAGUGCGUGAACUUCAACUUCAAC | |
| | GGCCUUACCGGCACCGGCGUGCUGACCGAGAGCAACAAGAAAUUCC | |
| | UGCCCUUUUGCCAGUUCGGCCGGGACAUCGCCGACACCACCGACGC | |
| | UGUGCGGGAUCCCCAGACCCUGGAGAUCCUGGACAUCACCCCUUGC | |
| | AGCUUCGGCGGCUCUGGCGGAGGCAGCAUCCUGGCCAUCUACAGCA | |
| | CCGUGGCCAGCAGCCUGGUGCUGCUGGUGAGCCUGGGCGCCAUCAG | |
| | CUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVCRSS | 29 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVGSGGGSGGGSGGGS | |
| | GGGSGGQPTESIVRFPNITNLCPFGEVENATRFASVYAWNRKRISN | |
| | CVADYSVLYNSASFSTFKCYGVSPTKLNDLCFTNVYADSFVIRGDE | |
| | VRQIAPGQTGKIADYNYKLPDDFTGCVIAWNSNNLDSKVGGNYNYL | |
| | YRLFRKSNLKPFERDISTEIYQAGSTPCNGVEGENCYFPLQSYGFQ | |
| | PTNGVGYQPYRVVVLSFELLHAPATVCGPKKSTNLVKNKCVNENEN | |
| | GLTGTGVLTESNKKFLPFCQFGRDIADTTDAVRDPQTLEILDITPC | |
| | SFGGsgggsilaiystvasslvllvslgaisf | |
| PolyA tail | 100 nt | |

SARS-CoV-2 S1 Subunit Truncated and Linked to Transmembrane
Domain (S1-666-TM)

| SEQ ID NO: 149 consists of from 5′ end to 3′ end: 5′ UTR SEQ ID NO: 2, | | 149 |
|---|---|---|
| mRNA ORF SEQ ID NO: 150 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCGU | 150 |
| Construct | GAACCUGACCACCCGGACCCAGCUGCCACCAGCCUACACCAACAGCU | |
| (excluding the stop | UCACCCGGGGCGUCUACUACCCCGACAAGGUGUUCCGGAGCAGCGUC | |
| codon) | CUGCACAGCACCCAGGACCUGUUCCUGCCCUUCUUCAGCAACGUGAC | |
| | CUGGUUCCACGCCAUCCACGUGAGCGGCACCAACGGCACCAAGCGGU | |
| | UCGACAACCCCGUGCUGCCCUUCAACGACGGCGUGUACUUCGCCAGC | |
| | ACCGAGAAGAGCAACAUCAUCCGGGGCUGGAUCUUCGGCACCACCCU | |
| | GGACAGCAAGACCCAGAGCCUGCUGAUCGUGAAUAACGCCACCAACG | |
| | UGGUGAUCAAGGUGUGCGAGUUCCAGUUCUGCAACGACCCCUUCCUG | |
| | GGCGUGUACUACCACAAGAACAACAAGAGCUGGAUGGAGAGCGAGUU | |
| | CCGGGUGUACAGCAGCGCCAACAACUGCACCUUCGAGUACGUGAGCC | |
| | AGCCCUUCCUGAUGGACCUGGAGGGCAAGCAGGGCAACUUCAAGAAC | |
| | CUGCGGGAGUUCGUGUUCAAGAACAUCGACGGCUACUUCAAGAUCUA | |
| | CAGCAAGCACACCCCAAUCAACCUGGUGCGGGAUCUGCCCCAGGGCU | |
| | UCUCAGCCCUGGAGCCCCUGGUGGACCUGCCCAUCGGCAUCAACAUC | |
| | ACCCGGUUCCAGACCCUGCUGGCCCUGCACCGGAGCUACCUGACCCC | |
| | AGGCGACAGCAGCAGCGGGUGGACAGCAGGCGCGGCUGCUUACUACG | |
| | UGGGCUACCUGCAGCCCCGGACCUUCCUGCUGAAGUACAACGAGAAC | |
| | GGCACCAUCACCGACGCCGUGGACUGCGCCCUGGACCCUCUGAGCGA | |
| | GACCAAGUGCACCCUGAAGAGCUUCACCGUGGAGAAGGGCAUCUACC | |
| | AGACCAGCAACUUCCGGGUGCAGCCCACCGAGAGCAUCGUGCGGUUC | |
| | CCCAACAUCACCAACCUGUGCCCCUUCGGCGAGGUGUUCAACGCCAC | |
| | CCGGUUCGCCAGCGUGUACGCCUGGAACCGGAAGCGGAUCAGCAACU | |
| | GCGUGGCCGACUACAGCGUGCUGUACAACAGCGCCAGCUUCAGCACC | |
| | UUCAAGUGCUACGGCGUGAGCCCCACCAAGCUGAACGACCUGUGCUU | |
| | CACCAACGUGUACGCCGACAGCUUCGUGAUCCGUGGCGACGAGGUGC | |
| | GGCAGAUCGCACCCGGCCAGACAGGCAAGAUCGCCGACUACAACUAC | |
| | AAGCUGCCCGACGACUUCACCGGCUGCGUGAUCGCCUGGAACAGCAA | |
| | CAACCUCGACAGCAAGGUGGGCGGCAACUACAACUACCUGUACCGGC | |
| | UGUUCCGGAAGAGCAACCUGAAGCCCUUCGAGCGGGACAUCAGCACC | |
| | GAGAUCUACCAAGCCGGCUCCACCCCUUGCAACGGCGUGGAGGGCUU | |
| | CAACUGCUACUUCCCUCUGCAGAGCUACGGCUUCCAGCCCACCAACG | |
| | GCGUGGGCUACCAGCCCUACCGGGUGGUGGUGCUGAGCUUCGAGCUG | |
| | CUGCACGCCCCAGCCACCGUGUGUGGCCCCAAGAAGAGCACCAACCU | |
| | GGUGAAGAACAAGUGCGUGAACUUCAACUUCAACGGCCUUACCGGCA | |
| | CCGGCGUGCUGACCGAGAGCAACAAGAAAUUCCUGCCCUUUCAGCAG | |
| | UUCGGCCGGGACAUCGCCGACACCACCGACGCUGUGCGGGAUCCCCA | |
| | GACCCUGGAGAUCCUGGACAUCACCCCUUGCAGCUUCGGCGGCGUGA | |
| | GCGUGAUCACCCCAGGCACCAACACCAGCAACCAGGUGGCCGUGCUG | |
| | UACCAGGACGUGAACUGCACCGAGGUGCCCGUGGCCAUCCACGCCGA | |
| | CCAGCUGACACCCACCUGGCGGGUCUACAGCACCGGCAGCAACGUGU | |
| | UCCAGACCCGGGCCGGUUGCCUGAUCGGCGCCGAGCACGUGAACAAC | |
| | AGCUACGAGUGCGACAUCCCCAUCGGCGCCGGCAUCUGUGCCAGCUA | |
| | CCAGACCCAGACCAAUUCA | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSSV | 151 |
| acid sequence | LHSTQDLFLPFFSNVTWFHAIHVSGTNGTKRFDNPVLPENDGVYFAS | |
| | TEKSNIIRGWIFGTTLDSKTQSLLIVNNATNVVIKVCEFQFCNDPFL | |
| | GVYYHKNNKSWMESEFRVYSSANNCTFEYVSQPFLMDLEGKQGNFKN | |
| | LREFVFKNIDGYFKIYSKHTPINLVRDLPQGFSALEPLVDLPIGINI | |
| | TRFQTLLALHRSYLTPGDSSSGWTAGAAAYYVGYLQPRTELLKYNEN | |
| | GTITDAVDCALDPLSETKCTLKSFTVEKGIYQTSNERVQPTESIVRE | |
| | PNITNLCPFGEVENATRFASVYAWNRKRISNCVADYSVLYNSASFST | |
| | FKCYGVSPTKLNDLCFTNVYADSFVIRGDEVRQIAPGQTGKIADYNY | |
| | KLPDDFTGCVIAWNSNNLDSKVGGNYNYLYRLFRKSNLKPFERDIST | |
| | EIYQAGSTPCNGVEGENCYFPLQSYGFQPTNGVGYQPYRVVVLSFEL | |
| | LHAPATVCGPKKSTNLVKNKCVNFNFNGLTGTGVLTESNKKELPFQQ | |
| | FGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNN | |
| | SYECDIPIGAGICASYQTQTNS | |
| PolyA tail | 100 nt | |

TABLE 4A
Subunit Antigen RBD Deletions

| | SEQ ID NO: | |
|---|---|---|
| Name | mRNA ORF | Protein |
| SARS-CoV-2 S1 Subunit with Deletion of RBD | 31 | 32 |
| and Glycan Added to S2 Subunit | | |
| SARS-CoV-2 S1 Subunit with Deletion of RBD | 34 | 35 |
| and NTD and Glycan Added to S2 Subunit | | |

TABLE 4B
Subunit Antigen RBD Deletions
SARS-CoV-2 S1 Subunit with Deletion of RBD and Glycan Added to S2 Subunit

| SEQ ID NO: 30 consists of from 5′ end to 3′ end: 5′ UTR SEQ ID NO: 2, | | 30 |
|---|---|---|
| mRNA ORF SEQ ID NO: 31 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 31 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCGGCGGCAGCGGC | |
| | GGCGUGAGCGUGAUCACCCCAGGCACCAACACCAGCAACCAGGUGG | |
| | CCGUGCUGUACCAGGACGUGAACUGCACCGAGGUGCCCGUGGCCAU | |
| | CCACGCCGACCAGCUGACACCCACCUGGCGGGUCUACAGCACCGGC | |
| | AGCAACGUGUUCCAGACCCGGGCCGGUUGCCUGAUCGGCGCCGAGC | |
| | ACGUGAACAACAGCUACGAGUGCGACAUCCCCAUCGGCGCCGGCAU | |
| | CUGUGCCAGCUACCAGACCCAGACCAAUUCACCCCGGAGGGCAAGG | |
| | AGCGUGGCCAGCCAGAGCAUCAUCGCCUACACCAUGAGCCUGGGCG | |
| | CCGAGAACAGCGUGGCCUACAGCAACAACAGCAUCGCCAUCCCCAC | |
| | CAACUUCACCAUCAGCGUGACCACCGAGAUUCUGCCCGUGAGCAUG | |
| | ACCAAGACCAGCGUGGACUGCACCAUGUACAUCUGCGGCGACAGCA | |
| | CCGAGUGCAGCAACCUGCUGCUGCAGUACGGCAGCUUCUGCACCCA | |
| | GCUGAACCGGGCCCUGACCGGCAUCGCCGUGGAGCAGGACAAGAAC | |
| | ACCCAGGAGGUGUUCGCCCAGGUGAAGCAGAUCUACAAGACCCCUC | |
| | CCAUCAAGGACUUCGGCGGCUUCAACUUCAGCCAGAUCCUGCCCGA | |
| | CCCCAGCAAGCCCAGCAAGCGGAGCUUCAUCGAGGACCUGCUGUUC | |
| | AACAAGGUGACCCUAGCCGACGCCGGCUUCAUCAAGCAGUACGGCG | |
| | ACUGCCUCGGCGACAUAGCCGCCCGGGACCUGAUCUGCGCCCAGAA | |
| | GUUCAACGGCCUGACCGUGCUGCCUCCCCUGCUGACCGACGAGAUG | |
| | AUCGCCCAGUACACCAGCGCCCUGUUAGCCGGAACCAUCACCAGCG | |
| | GCUGGACUUUCGGCGCUGGAGCCGCUCUGCAGAUCCCCUUCGCCAU | |
| | GCAGAUGGCCUACCGGUUCAACGGCAUCGGCGUGACCCAGAACGUG | |
| | CUGUACGAGAACCAGAAGCUGAUCGCCAACCAGUUCAACAGCGCCA | |
| | UCGGCAAGAUCCAGGACAGCCUGAGCAGCACCGCUAGCGCCCUGGG | |
| | CAAGCUGCAGGACGUGGUGAACCAGAACGCCCAGGCCCUGAACACC | |
| | CUGGUGAAGCAGCUGAGCAGCAACUUCGGCGCCAUCAGCAGCGUGC | |
| | UGAACGACAUCCUGAGCCGGCUGGACCCUCCCAACGCCACCGUGCA | |
| | GAUCGACCGGCUGAUCACUGGCCGGCUGCAGAGCCUGCAGACCUAC | |
| | GUGACCCAGCAGCUGAUCCGGGCCGCCGAGAUUCGGGCCAGCGCCA | |
| | ACCUGGCCGCCACCAAGAUGAGCGAGUGCGUGCUGGGCCAGAGCAA | |
| | GCGGGUGGACUUCUGCGGCAAGGGCUACCACCUGAUGAGCUUUCCC | |
| | CAGAGCGCACCCCACGGAGUGGUGUUCCUGCACGUGACCUACGUGC | |
| | CCGCCCAGGAGAAGAACUUCACCACCGCCCCAGCCAUCUGCCACGA | |
| | CGGCAAGGCCCACUUUCCCCGGGAGGGCGUGUUCGUGAGCAACGGC | |
| | ACCCACUGGUUCGUGACCCAGCGGAACUUCUACGAGCCCCAGAUCA | |
| | UCACCACCGACAACACCUUCGUGAGCGGCAACUGCGACGUGGUGAU | |
| | CGGCAUCGUGAACAACACCGUGUACGAUCCCCUGCAGCCCGAGCUG | |
| | GACAGCUUCAAGGAGGAGCUGGACAAGUACUUCAAGAAUCACACCA | |
| | GCCCCGACGUGGACCUGGGCGACAUCAGCGGCAUCAACGCCAGCGU | |
| | GGUGAACAUCCAGAAGGAGAUCGAUCGGCUGAACGAGGUGGCCAAG | |
| | AACCUGAACGAGAGCCUGAUCGACCUGCAGGAGCUGGGCAAGUACG | |
| | AGCAGUACAUCAAGUGGCCCUGGUACAUCUGGCUGGGCUUCAUCGC | |
| | CGGCCUGAUCGCCAUCGUGAUGGUGACCAUCAUGCUGUGCUGCAUG | |
| | ACCAGCUGCUGCAGCUGCCUGAAGGGCUGUUGCAGCUGCGGCAGCU | |
| | GCUGCAAGUUCGACGAGGACGACAGCGAGCCCGUGCUGAAGGGCGU | |
| | GAAGCUGCACUACACC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 32 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNFGGSG | |
| | GVSVITPGTNTSNQVAVLYQDVNCTEVPVAIHADQLTPTWRVYSTG | |
| | SNVFQTRAGCLIGAEHVNNSYECDIPIGAGICASYQTQTNSPRRAR | |
| | SVASQSIIAYTMSLGAENSVAYSNNSIAIPTNFTISVTTEILPVSM | |
| | TKTSVDCTMYICGDSTECSNLLLQYGSFCTQLNRALTGIAVEQDKN | |
| | TQEVFAQVKQIYKTPPIKDFGGFNFSQILPDPSKPSKRSFIEDLLF | |
| | NKVTLADAGFIKQYGDCLGDIAARDLICAQKFNGLTVLPPLLTDEM | |
| | IAQYTSALLAGTITSGWTFGAGAALQIPFAMQMAYRENGIGVTQNV | |
| | LYENQKLIANQFNSAIGKIQDSLSSTASALGKLQDVVNQNAQALNT | |
| | LVKQLSSNFGAISSVLNDILSRLDPPNATVQIDRLITGRLQSLQTY | |
| | VTQQLIRAAEIRASANLAATKMSECVLGQSKRVDFCGKGYHLMSFP | |
| | QSAPHGVVFLHVTYVPAQEKNFTTAPAICHDGKAHFPREGVFVSNG | |
| | THWFVTQRNFYEPQIITTDNTFVSGNCDVVIGIVNNTVYDPLQPEL | |
| | DSFKEELDKYFKNHTSPDVDLGDISGINASVVNIQKEIDRLNEVAK | |
| | NLNESLIDLQELGKYEQYIKWPWYIWLGFIAGLIAIVMVTIMLCCM | |
| | TSCCSCLKGCCSCGSCCKFDEDDSEPVLKGVKLHYT | |
| PolyA tail | 100 nt | |

SARS-CoV-2 S1 Subunit with Deletion of RBD and
NTD and Glycan Added to S2 Subunit

| SEQ ID NO: 33 consists of from 5′ end to 3′ end: 5′ UTR SEQ ID NO: 2, | | 33 |
|---|---|---|
| mRNA ORF SEQ ID NO: 34 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGGGCA | 34 |
| Construct | CCAUCACCGACGCCGUGGACUGCGCCCUGGACCCUCUGAGCGAGAC | |
| (excluding the stop | CAAGUGCACCCUGAAGAGCUUCACCGUGGAGAAGGGCAUCUACCAG | |
| codon) | ACCAGCAACUUCGGCGGCAGCGGCGGCGUGAGCGUGAUCACCCCAG | |
| | GCACCAACACCAGCAACCAGGUGGCCGUGCUGUACCAGGACGUGAA | |
| | CUGCACCGAGGUGCCCGUGGCCAUCCACGCCGACCAGCUGACACCC | |
| | ACCUGGCGGGUCUACAGCACCGGCAGCAACGUGUUCCAGACCCGGG | |
| | CCGGUUGCCUGAUCGGCGCCGAGCACGUGAACAACAGCUACGAGUG | |
| | CGACAUCCCCAUCGGCGCCGGCAUCUGUGCCAGCUACCAGACCCAG | |
| | ACCAAUUCACCCCGGAGGGCAAGGAGCGUGGCCAGCCAGAGCAUCA | |
| | UCGCCUACACCAUGAGCCUGGGCGCCGAGAACAGCGUGGCCUACAG | |
| | CAACAACAGCAUCGCCAUCCCCACCAACUUCACCAUCAGCGUGACC | |
| | ACCGAGAUUCUGCCCGUGAGCAUGACCAAGACCAGCGUGGACUGCA | |
| | CCAUGUACAUCUGCGGCGACAGCACCGAGUGCAGCAACCUGCUGCU | |
| | GAACUACACCAGCUUCUGCACCCAGCUGAACCGGGCCCUGACCGGC | |
| | AUCGCCGUGGAGCAGGACAAGAACACCCAGGAGGUGUUCGCCCAGG | |
| | UGAAGCAGAUCUACAAGACCCCUCCCAUCAAGGACUUCGGCGGCUU | |
| | CAACUUCAGCCAGAUCCUGCCCGACCCCAGCAAGCCCAGCAAGCGG | |
| | AGCUUCAUCGAGGACCUGCUGUUCAACAAGGUGACCCUAGCCGACG | |
| | CCGGCUUCAUCAAGCAGUACGGCGACUGCCUCGGCGACAUAGCCGC | |
| | CCGGGACCUGAUCUGCGCCCAGAAGUUCAACGGCCUGACCGUGCUG | |
| | CCUCCCCUGCUGACCGACGAGAUGAUCGCCCAGUACACCAGCGCCC | |
| | UGUUAGCCGGAACCAUCACCAGCGGCUGGACUUUCGGCGCUGGAGC | |
| | CGCUCUGCAGAUCCCCUUCGCCAUGCAGAUGGCCUACCGGUUCAAC | |
| | GGCAUCGGCGUGACCCAGAACGUGCUGUACGAGAACCAGAAGCUGA | |
| | UCGCCAACCAGUUCAACAGCGCCAUCGGCAAGAUCCAGGACAGCCU | |
| | GAGCAGCACCGCUAGCGCCCUGGGCAAGCUGCAGGACGUGGUGAAC | |
| | CAGAACGCCCAGGCCCUGAACACCCUGGUGAAGCAGCUGAGCAGCA | |
| | ACUUCGGCGCCAUCAGCAGCGUGCUGAACGACAUCCUGAGCCGGCU | |
| | GGACCCUCCCAACGCCACCGUGCAGAUCGACCGGCUGAUCACUGGC | |
| | CGGCUGCAGAGCCUGCAGACCUACGUGACCCAGCAGCUGAUCCGGG | |
| | CCGCCGAGAUUCGGGCCAGCGCCAACCUGGCCGCCACCAAGAUGAG | |
| | CGAGUGCGUGCUGGGCCAGAGCAAGCGGGUGGACUUCUGCGGCAAG | |
| | GGCUACCACCUGAUGAGCUUUCCCCAGAGCGCACCCCACGGAGUGG | |
| | UGUUCCUGCACGUGACCUACGUGCCCGCCCAGGAGAAGAACUUCAC | |
| | CACCGCCCCAGCCAUCUGCCACGACGGCAAGGCCCACUUUCCCCGG | |
| | GAGGGCGUGUUCGUGAGCAACGGCACCCACUGGUUCGUGACCCAGC | |
| | GGAACUUCUACGAGCCCCAGAUCAUCACCACCGACAACACCUUCGU | |
| | GAGCGGCAACUGCGACGUGGUGAUCGGCAUCGUGAACAACACCGUG | |
| | UACGAUCCCCUGCAGCCCGAGCUGGACAGCUUCAAGGAGGAGCUGG | |
| | ACAAGUACUUCAAGAAUCACACCAGCCCCGACGUGGACCUGGGCGA | |
| | CAUCAGCGGCAUCAACGCCAGCGUGGUGAACAUCCAGAAGGAGAUC | |
| | GAUCGGCUGAACGAGGUGGCCAAGAACCUGAACGAGAGCCUGAUCG | |
| | ACCUGCAGGAGCUGGGCAAGUACGAGCAGUACAUCAAGUGGCCCUG | |
| | GUACAUCUGGCUGGGCUUCAUCGCCGGCCUGAUCGCCAUCGUGAUG | |
| | GUGACCAUCAUGCUGUGCUGCAUGACCAGCUGCUGCAGCUGCCUGA | |
| | AGGGCUGUUGCAGCUGCGGCAGCUGCUGCAAGUUCGACGAGGACGA | |
| | CAGCGAGCCCGUGCUGAAGGGCGUGAAGCUGCACUACACC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQGTITDAVDCALDPLSETKCTLKSFTVEKGIYQ | 35 |
| acid sequence | TSNFGGSGGVSVITPGTNTSNQVAVLYQDVNCTEVPVAIHADQLTP | |
| | TWRVYSTGSNVFQTRAGCLIGAEHVNNSYECDIPIGAGICASYQTQ | |
| | TNSPRRARSVASQSIIAYTMSLGAENSVAYSNNSIAIPTNFTISVT | |
| | TEILPVSMTKISVDCTMYICGDSTECSNLLLNYTSFCTQLNRALTG | |
| | IAVEQDKNTQEVFAQVKQIYKTPPIKDFGGFNFSQILPDPSKPSKR | |
| | SFIEDLLFNKVTLADAGFIKQYGDCLGDIAARDLICAQKENGLTVL | |
| | PPLLTDEMIAQYTSALLAGTITSGWTFGAGAALQIPFAMQMAYREN | |
| | GIGVTQNVLYENQKLIANQFNSAIGKIQDSLSSTASALGKLQDVVN | |
| | QNAQALNTLVKQLSSNFGAISSVLNDILSRLDPPNATVQIDRLITG | |
| | RLQSLQTYVTQQLIRAAEIRASANLAATKMSECVLGQSKRVDFCGK | |
| | GYHLMSFPQSAPHGVVFLHVTYVPAQEKNFTTAPAICHDGKAHFPR | |
| | EGVFVSNGTHWFVTQRNFYEPQIITTDNTFVSGNCDVVIGIVNNTV | |
| | YDPLQPELDSFKEELDKYFKNHISPDVDLGDISGINASVVNIQKEI | |
| | DRLNEVAKNLNESLIDLQELGKYEQYIKWPWYIWLGFIAGLIAIVM | |
| | VTIMLCCMTSCCSCLKGCCSCGSCCKFDEDDSEPVLKGVKLHYT | |
| PolyA tail | 100 nt | |

### Chimeric S1-S2 Subunit Antigens

In some embodiments, a composition comprises an mRNA that encodes a chimeric protein, for example a chimeric S1-S2 protein with an S1 subunit from an S protein of one virus and an S2 subunit from an S protein of another, different virus. For example, an S2 subunit may be from SARS-CoV-2, while the S1 subunit may be from HKU As another example, an S2 subunit may be from SARS-CoV-2, while the S1 subunit may be from OC43. These chimeric proteins are likely to be opsonized by circulating antibodies specific to the S1 subunit of HKU1 or OC43 generated by previous exposures, promoting efficient uptake and cross-presentation of SARS-CoV-2 S2 subunit peptides to CD4⁺ T cells by macrophages and dendritic cells. Opsonization by circulating antibodies also promotes capture by follicular dendritic cells for presentation to B cells with receptors specific to SARS-CoV-2 S2 subunit epitopes. Non-limiting examples of chimeric S1/S2 subunit constructs and the mRNA encoding them are provided in Tables 5A and 5B below.

TABLE 5A
Chimeric S1 Subunit-S2 Subunit Antigens

| | SEQ ID NO: | |
|---|---|---|
| Name | mRNA ORF | Protein |
| SARS-CoV-2 S2 Subunit Linked to HKU1 S1 | 37 | 38 |
| Subunit | | |
| SARS-CoV-2 S2 Subunit Linked to OC43 S1 | 40 | 41 |
| Subunit | | |

TABLE 5B
Chimeric S1 Subunit-S2 Subunit Antigens
SARS-CoV-2 S2 Subunit Linked to HKU1 S1 Subunit

| SEQ ID NO: 36 consists of from 5′ end to 3′ end: 5′ UTR SEQ ID NO: 2, | | 36 |
|---|---|---|
| mRNA ORF SEQ ID NO: 37 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCCUGAUCAUCUUCAUCCUGCCCACCACCCUGGCCGUGAUCG | 37 |
| Construct | GCGACUUCAACUGCACCAACAGCUUCAUCAACGACUACAACAAGAC | |
| (excluding the stop | CAUCCCUCGGAUCAGCGAAGACGUGGUGGACGUGUCCCUGGGCCUG | |
| codon) | GGCACCUACUACGUGCUGAACCGGGUGUACCUGAACACCACACUGC | |
| | UGUUCACCGGCUACUUCCCCAAGAGCGGCGCCAACUUCCGGGACCU | |
| | GGCCCUGAAGGGCAGCAUCUACCUGAGCACCUUGUGGUACAAGCCU | |
| | CCCUUCCUGAGCGACUUCAAUAACGGCAUCUUCUCUAAGGUGAAGA | |
| | ACACCAAGCUGUACGUAAACAACACCCUGUACAGCGAGUUCAGCAC | |
| | CAUCGUGAUCGGCAGCGUGUUCGUCAACACCAGCUACACCAUCGUG | |
| | GUGCAGCCCCACAACGGCAUCCUGGAGAUCACCGCCUGCCAGUACA | |
| | CCAUGUGCGAGUACCCUCACACCGUGUGCAAGAGCAAGGGCUCCAU | |
| | CCGGAACGAGAGCUGGCACAUCGACAGCAGCGAGCCGCUGUGCCUG | |
| | UUCAAGAAGAACUUCACCUACAACGUGAGCGCCGACUGGCUGUACU | |
| | UCCACUUCUACCAGGAGCGGGGCGUGUUCUACGCCUACUACGCCGA | |
| | CGUGGGCAUGCCAACCACCUUCCUGUUCAGCCUGUACCUGGGCACC | |
| | AUCCUGAGCCACUACUACGUGAUGCCCCUGACCUGCAACGCCAUCA | |
| | GCUCAAACACCGACAACGAGACCCUGGAGUACUGGGUGACUCCACU | |
| | GAGCCGGCGGCAGUACCUGCUGAACUUCGACGAGCACGGCGUGAUC | |
| | ACCAACGCCGUGGACUGCGCCCUGGACCCUCUGAGCGAGACCAAGU | |
| | GCACCCUGAAGAGCUUCACCGUGGAGAAGGGCAUCUACCAGACCAG | |
| | CGGCUUCACCGUGAAGCCCGUAGCCACCGUGUACCGGCGGAUCCCC | |
| | AACCUGCCCGACUGCGACAUCGACAACUGGCUGAACAACGUCAGCG | |
| | UGCCCAGCCCACUGAACUGGGAGCGGCGGAUCUUCAGCAACUGCAA | |
| | CUUCAAUCUGAGCACCCUGCUGCGGCUGGUGCACGUGGACAGCUUC | |
| | AGCUGCAACAACCUGGACAAGAGCAAGAUCUUCGGUAGCUGCUUCA | |
| | ACAGCAUCACCGUGGACAAGUUCGCCAUCCCUAACCGGCGGCGGGA | |
| | CGAUCUGCAGCUGGGCAGCAGCGGCUUCCUGCAGAGCAGCAACUAC | |
| | AAGAUCGACAUCAGCAGCUCAAGCUGCCAGCUGUACUACAGCCUGC | |
| | CCCUGGUGAACGUGACCAUCAACAACUUCAACCCCAGCAGCUGGAA | |
| | CCGGCGGUACGGCUUCGGCAGCUUCAACCUGAGCAGCUACGACGUG | |
| | GUGUACAGCGACCACUGCUUCAGCGUGAACAGCGACUUCUGCCCCU | |
| | GUGCCGACCCUAGCGUGGUGAACAGCUGCGCCAAGAGCAAGCCUCC | |
| | CAGCGCCAUUUGCCCCGCCGGCACCAAGUACCGGCACUGCGACCUG | |
| | GACACCACCCUGUACGUGAAGAACUGGUGCCGGUGCAGCUGCCUGC | |
| | CCGACCCCAUCAGCACCUACAGCCCCAACACCUGUCCCCAGAAGAA | |
| | GGUGGUGGUGGGUAUCGGCGAGCACUGUCCCGGCCUGGGCAUCAAC | |
| | GAGGAGAAGUGCGGCACCCAGCUGAACCACAGCAGCUGCUUCUGUA | |
| | GCCCCGACGCCUUCCUGGGCUGGAGCUUCGACAGCUGCAUCAGCAA | |
| | CAACCGGUGCAACAUCUUUAGCAACUUCAUCUUCAACGGAAUCAAC | |
| | AGCGGCACCACCUGCAGCAACGACCUGCUGUAUAGCAACACCGAGA | |
| | UCAGCACCGGCGUGUGCGUGAACUACGACCUGUACGGCAUCACCGG | |
| | CCAGGGCAUCUUCAAGGAGGUGAGCGCCGCCUACUACAACAACUGG | |
| | CAGAACCUGCUGUACGACAGCAACGGCAACAUCAUCGGCUUCAAGG | |
| | ACUUUCUGACCAACAAGACCUACACCAUCCUGCCCUGCUACAGCGG | |
| | CGGCGUGAGCGUGAUCACCCCAGGCACCAACACCAGCAACCAGGUG | |
| | GCCGUGCUGUACCAGGACGUGAACUGCACCGAGGUGCCCGUGGCCA | |
| | UCCACGCCGACCAGCUGACACCCACCUGGCGGGUCUACAGCACCGG | |
| | CAGCAACGUGUUCCAGACCCGGGCCGGUUGCCUGAUCGGCGCCGAG | |
| | CACGUGAACAACAGCUACGAGUGCGACAUCCCCAUCGGCGCCGGCA | |
| | UCUGUGCCAGCUACCAGACCCAGACCAAUUCACCCCGGAGGGCAAG | |
| | GAGCGUGGCCAGCCAGAGCAUCAUCGCCUACACCAUGAGCCUGGGC | |
| | GCCGAGAACAGCGUGGCCUACAGCAACAACAGCAUCGCCAUCCCCA | |
| | CCAACUUCACCAUCAGCGUGACCACCGAGAUUCUGCCCGUGAGCAU | |
| | GACCAAGACCAGCGUGGACUGCACCAUGUACAUCUGCGGCGACAGC | |
| | ACCGAGUGCAGCAACCUGCUGCUGCAGUACGGCAGCUUCUGCACCC | |
| | AGCUGAACCGGGCCCUGACCGGCAUCGCCGUGGAGCAGGACAAGAA | |
| | CACCCAGGAGGUGUUCGCCCAGGUGAAGCAGAUCUACAAGACCCCU | |
| | CCCAUCAAGGACUUCGGCGGCUUCAACUUCAGCCAGAUCCUGCCCG | |
| | ACCCCAGCAAGCCCAGCAAGCGGAGCUUCAUCGAGGACCUGCUGUU | |
| | CAACAAGGUGACCCUAGCCGACGCCGGCUUCAUCAAGCAGUACGGC | |
| | GACUGCCUCGGCGACAUAGCCGCCCGGGACCUGAUCUGCGCCCAGA | |
| | AGUUCAACGGCCUGACCGUGCUGCCUCCCCUGCUGACCGACGAGAU | |
| | GAUCGCCCAGUACACCAGCGCCCUGUUAGCCGGAACCAUCACCAGC | |
| | GGCUGGACUUUCGGCGCUGGAGCCGCUCUGCAGAUCCCCUUCGCCA | |
| | UGCAGAUGGCCUACCGGUUCAACGGCAUCGGCGUGACCCAGAACGU | |
| | GCUGUACGAGAACCAGAAGCUGAUCGCCAACCAGUUCAACAGCGCC | |
| | AUCGGCAAGAUCCAGGACAGCCUGAGCAGCACCGCUAGCGCCCUGG | |
| | GCAAGCUGCAGGACGUGGUGAACCAGAACGCCCAGGCCCUGAACAC | |
| | CCUGGUGAAGCAGCUGAGCAGCAACUUCGGCGCCAUCAGCAGCGUG | |
| | CUGAACGACAUCCUGAGCCGGCUGGACCCUCCCGAGGCCGAGGUGC | |
| | AGAUCGACCGGCUGAUCACUGGCCGGCUGCAGAGCCUGCAGACCUA | |
| | CGUGACCCAGCAGCUGAUCCGGGCCGCCGAGAUUCGGGCCAGCGCC | |
| | AACCUGGCCGCCACCAAGAUGAGCGAGUGCGUGCUGGGCCAGAGCA | |
| | AGCGGGUGGACUUCUGCGGCAAGGGCUACCACCUGAUGAGCUUUCC | |
| | CCAGAGCGCACCCCACGGAGUGGUGUUCCUGCACGUGACCUACGUG | |
| | CCCGCCCAGGAGAAGAACUUCACCACCGCCCCAGCCAUCUGCCACG | |
| | ACGGCAAGGCCCACUUUCCCCGGGAGGGCGUGUUCGUGAGCAACGG | |
| | CACCCACUGGUUCGUGACCCAGCGGAACUUCUACGAGCCCCAGAUC | |
| | AUCACCACCGACAACACCUUCGUGAGCGGCAACUGCGACGUGGUGA | |
| | UCGGCAUCGUGAACAACACCGUGUACGAUCCCCUGCAGCCCGAGCU | |
| | GGACAGCUUCAAGGAGGAGCUGGACAAGUACUUCAAGAAUCACACC | |
| | AGCCCCGACGUGGACCUGGGCGACAUCAGCGGCAUCAACGCCAGCG | |
| | UGGUGAACAUCCAGAAGGAGAUCGAUCGGCUGAACGAGGUGGCCAA | |
| | GAACCUGAACGAGAGCCUGAUCGACCUGCAGGAGCUGGGCAAGUAC | |
| | GAGCAGUACAUCAAGUGGCCCUGGUACAUCUGGCUGGGCUUCAUCG | |
| | CCGGCCUGAUCGCCAUCGUGAUGGUGACCAUCAUGCUGUGCUGCAU | |
| | GACCAGCUGCUGCAGCUGCCUGAAGGGCUGUUGCAGCUGCGGCAGC | |
| | UGCUGCAAGUUCGACGAGGACGACAGCGAGCCCGUGCUGAAGGGCG | |
| | UGAAGCUGCACUACACC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFLIIFILPTTLAVIGDENCINSFINDYNKTIPRISEDVVDVSLGL | 38 |
| acid sequence | GTYYVLNRVYLNTTLLFTGYFPKSGANFRDLALKGSIYLSTLWYKP | |
| | PFLSDENNGIFSKVKNTKLYVNNTLYSEFSTIVIGSVFVNTSYTIV | |
| | VQPHNGILEITACQYTMCEYPHTVCKSKGSIRNESWHIDSSEPLCL | |
| | FKKNFTYNVSADWLYFHFYQERGVFYAYYADVGMPTTFLFSLYLGT | |
| | ILSHYYVMPLICNAISSNIDNETLEYWVTPLSRRQYLLNFDEHGVI | |
| | TNAVDCALDPLSETKCTLKSFTVEKGIYQTSGFTVKPVATVYRRIP | |
| | NLPDCDIDNWLNNVSVPSPLNWERRIFSNCNFNLSTLLRLVHVDSF | |
| | SCNNLDKSKIFGSCENSITVDKFAIPNRRRDDLQLGSSGFLQSSNY | |
| | KIDISSSSCQLYYSLPLVNVTINNENPSSWNRRYGFGSFNLSSYDV | |
| | VYSDHCFSVNSDFCPCADPSVVNSCAKSKPPSAICPAGTKYRHCDL | |
| | DTTLYVKNWCRCSCLPDPISTYSPNTCPQKKVVVGIGEHCPGLGIN | |
| | EEKCGTQLNHSSCFCSPDAFLGWSFDSCISNNRCNIFSNFIFNGIN | |
| | SGTTCSNDLLYSNTEISTGVCVNYDLYGITGQGIFKEVSAAYYNNW | |
| | QNLLYDSNGNIIGFKDFLINKTYTILPCYSGGVSVITPGTNTSNQV | |
| | AVLYQDVNCTEVPVAIHADQLTPTWRVYSTGSNVFQTRAGCLIGAE | |
| | HVNNSYECDIPIGAGICASYQTQTNSPRRARSVASQSIIAYTMSLG | |
| | AENSVAYSNNSIAIPTNFTISVTTEILPVSMTKTSVDCTMYICGDS | |
| | TECSNLLLQYGSFCTQLNRALTGIAVEQDKNTQEVFAQVKQIYKTP | |
| | PIKDFGGFNFSQILPDPSKPSKRSFIEDLLENKVTLADAGFIKQYG | |
| | DCLGDIAARDLICAQKFNGLTVLPPLLTDEMIAQYTSALLAGTITS | |
| | GWTFGAGAALQIPFAMQMAYRENGIGVTQNVLYENQKLIANQFNSA | |
| | IGKIQDSLSSTASALGKLQDVVNQNAQALNTLVKQLSSNFGAISSV | |
| | LNDILSRLDPPEAEVQIDRLITGRLQSLQTYVTQQLIRAAEIRASA | |
| | NLAATKMSECVLGQSKRVDFCGKGYHLMSFPQSAPHGVVFLHVTYV | |
| | PAQEKNFTTAPAICHDGKAHFPREGVFVSNGTHWFVTQRNFYEPQI | |
| | ITTDNTFVSGNCDVVIGIVNNTVYDPLQPELDSFKEELDKYFKNHT | |
| | SPDVDLGDISGINASVVNIQKEIDRLNEVAKNLNESLIDLQELGKY | |
| | EQYIKWPWYIWLGFIAGLIAIVMVTIMLCCMTSCCSCLKGCCSCGS | |
| | CCKFDEDDSEPVLKGVKLHYT | |
| PolyA tail | 100 nt | |

SARS-CoV-2 S2 Subunit Linked to OC43 S1 Subunit

| | |
|---|---|
| SEQ ID NO: 39 consists of from 5′ end to 3′ end: 5′ UTR SEQ ID NO: 2, | 39 |
| mRNA ORF SEQ IDNO: 40 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCCUGAUCCUGCUGAUCAGCCUGCCCACCGCCUUCGCCGUGA | 40 |
| Construct | UCGGCGACCUGAAGUGCACCAGCGACAACAUCAACGACAAGGACAC | |
| (excluding the stop | CGGCCCACCACCCAUCAGCACCGACACCGUGGACGUGACCAACGGC | |
| codon) | CUGGGCACCUACUACGUGCUGGACCGGGUGUACCUGAACACCACCC | |
| | UGUUCCUGAACGGCUACUACCCCACCAGCGGCAGCACCUACCGGAA | |
| | UAUGGCCCUGAAGGGCAGCGUGCUGCUGAGCCGGCUGUGGUUCAAG | |
| | CCACCAUUCCUGAGCGACUUCAUCAACGGAAUCUUCGCCAAGGUGA | |
| | AGAACACCAAGGUGAUCAAGGACCGGGUGAUGUACAGCGAGUUCCC | |
| | CGCCAUCACCAUUGGCAGUACCUUCGUGAACACCAGCUACAGCGUG | |
| | GUGGUGCAGCCCCGGACCAUCAACAGCACCCAGGACGGCGACAACA | |
| | AGCUGCAGGGCCUGCUGGAGGUGAGCGUGUGCCAGUACAACAUGUG | |
| | CGAGUACCCUCAGACCAUCUGCCACCCCAACCUGGGCAACCACCGG | |
| | AAGGAGCUGUGGCACCUGGACACCGGCGUGGUGAGCUGCCUGUACA | |
| | AGCGGAACUUCACCUACGACGUAAACGCCGACUACCUGUACUUCCA | |
| | CUUCUACCAGGAGGGCGGCACCUUCUACGCCUACUUCACCGACACG | |
| | GGCGUGGUGACCAAGUUCCUGUUCAACGUGUACCUGGGCAUGGCCC | |
| | UGAGCCACUACUACGUGAUGCCCCUGACCUGUAACAGCAAGCUGAC | |
| | CCUGGAGUACUGGGUGACCCCUCUGACCAGCCGGCAGUACCUGCUG | |
| | GCCUUCAACCAGGACGGCAUCAUCUUCAACGCCGUGGACUGCGCCC | |
| | UGGACCCUCUGAGCGAGACCAAGUGCACCCUGAAGAGCUUCACCGU | |
| | GGAGAAGGGCAUCUACCAGACCAACGGCUACACCGUGCAGCCCAUC | |
| | GCCGACGUGUACCGGCGGAAGCCCAACCUGCCCAACUGCAACAUCG | |
| | AGGCCUGGCUGAACGACAAGAGCGUGCCCUCGCCCCUGAACUGGGA | |
| | GCGGAAGACCUUCAGCAACUGCAACUUCAACAUGAGCAGUCUGAUG | |
| | AGCUUCAUCCAGGCCGACAGCUUCACCUGCAACAACAUCGACGCCG | |
| | CCAAGAUCUACGGCAUGUGCUUCAGCAGCAUCACCAUCGACAAGUU | |
| | UGCCAUCCCCAACGGCCGGAAGGUGGACCUGCAGCUGGGCAACCUG | |
| | GGCUACCUGCAGAGCUUCAACUACCGGAUCGACACCACCGCCACCU | |
| | CUUGCCAGCUGUACUACAACCUGCCCGCCGCCAACGUGAGCGUGAG | |
| | CCGGUUCAACCCCAGCACCUGGAACAAGCGGUUCGGCUUCAUUGAG | |
| | GACAGCGUGUUCAAACCCCGGCCCGCAGGAGUACUGACCAACCACG | |
| | ACGUGGUGUACGCCCAGCACUGCUUCAAGGCACCCAAGAACUUCUG | |
| | CCCCUGCAAGCUGAACGGCAGCUGUGUGGGCUCUGGCCCCGGUAAG | |
| | AACAACGGCAUAGGGACUUGCCCGGCAGGGACCAACUACCUGACCU | |
| | GCGACAACCUGUGCACACCCGACCCCAUCACCUUCACCGGCACCUA | |
| | CAAGUGUCCCCAGACCAAGAGCCUGGUGGGCAUCGGCGAGCACUGC | |
| | AGCGGCCUGGCCGUGAAGAGCGACUACUGCGGCGGCAACAGCUGCA | |
| | CCUGUCGGCCCCAGGCCUUCCUGGGCUGGAGCGCCGACAGCUGCCU | |
| | GCAGGGCGACAAGUGCAACAUCUUUGCCAACUUCAUCCUGCACGAC | |
| | GUGAACAGCGGCCUGACCUGCAGCACCGACCUGCAGAAGGCCAACA | |
| | CCGACAUCAUCCUGGGCGUGUGCGUGAACUACGACUUGUACGGCAU | |
| | CCUGGGCCAGGGCAUCUUCGUGGAGGUGAACGCCACCUACUACAAC | |
| | AGCUGGCAGAACCUGCUGUACGACAGCAACGGCAACCUGUACGGCU | |
| | UCCGGGACUACAUCAUCAACCGGACCUUCAUGAUCCGGAGCUGCUA | |
| | CAGCGGCGGCGUGAGCGUGAUCACCCCAGGCACCAACACCAGCAAC | |
| | CAGGUGGCCGUGCUGUACCAGGACGUGAACUGCACCGAGGUGCCCG | |
| | UGGCCAUCCACGCCGACCAGCUGACACCCACCUGGCGGGUCUACAG | |
| | CACCGGCAGCAACGUGUUCCAGACCCGGGCCGGUUGCCUGAUCGGC | |
| | GCCGAGCACGUGAACAACAGCUACGAGUGCGACAUCCCCAUCGGCG | |
| | CCGGCAUCUGUGCCAGCUACCAGACCCAGACCAAUUCACCCCGGAG | |
| | GGCAAGGAGCGUGGCCAGCCAGAGCAUCAUCGCCUACACCAUGAGC | |
| | CUGGGCGCCGAGAACAGCGUGGCCUACAGCAACAACAGCAUCGCCA | |
| | UCCCCACCAACUUCACCAUCAGCGUGACCACCGAGAUUCUGCCCGU | |
| | GAGCAUGACCAAGACCAGCGUGGACUGCACCAUGUACAUCUGCGGC | |
| | GACAGCACCGAGUGCAGCAACCUGCUGCUGCAGUACGGCAGCUUCU | |
| | GCACCCAGCUGAACCGGGCCCUGACCGGCAUCGCCGUGGAGCAGGA | |
| | CAAGAACACCCAGGAGGUGUUCGCCCAGGUGAAGCAGAUCUACAAG | |
| | ACCCCUCCCAUCAAGGACUUCGGCGGCUUCAACUUCAGCCAGAUCC | |
| | UGCCCGACCCCAGCAAGCCCAGCAAGCGGAGCUUCAUCGAGGACCU | |
| | GCUGUUCAACAAGGUGACCCUAGCCGACGCCGGCUUCAUCAAGCAG | |
| | UACGGCGACUGCCUCGGCGACAUAGCCGCCCGGGACCUGAUCUGCG | |
| | CCCAGAAGUUCAACGGCCUGACCGUGCUGCCUCCCCUGCUGACCGA | |
| | CGAGAUGAUCGCCCAGUACACCAGCGCCCUGUUAGCCGGAACCAUC | |
| | ACCAGCGGCUGGACUUUCGGCGCUGGAGCCGCUCUGCAGAUCCCCU | |
| | UCGCCAUGCAGAUGGCCUACCGGUUCAACGGCAUCGGCGUGACCCA | |
| | GAACGUGCUGUACGAGAACCAGAAGCUGAUCGCCAACCAGUUCAAC | |
| | AGCGCCAUCGGCAAGAUCCAGGACAGCCUGAGCAGCACCGCUAGCG | |
| | CCCUGGGCAAGCUGCAGGACGUGGUGAACCAGAACGCCCAGGCCCU | |
| | GAACACCCUGGUGAAGCAGCUGAGCAGCAACUUCGGCGCCAUCAGC | |
| | AGCGUGCUGAACGACAUCCUGAGCCGGCUGGACCCUCCCGAGGCCG | |
| | AGGUGCAGAUCGACCGGCUGAUCACUGGCCGGCUGCAGAGCCUGCA | |
| | GACCUACGUGACCCAGCAGCUGAUCCGGGCCGCCGAGAUUCGGGCC | |
| | AGCGCCAACCUGGCCGCCACCAAGAUGAGCGAGUGCGUGCUGGGCC | |
| | AGAGCAAGCGGGUGGACUUCUGCGGCAAGGGCUACCACCUGAUGAG | |
| | CUUUCCCCAGAGCGCACCCCACGGAGUGGUGUUCCUGCACGUGACC | |
| | UACGUGCCCGCCCAGGAGAAGAACUUCACCACCGCCCCAGCCAUCU | |
| | GCCACGACGGCAAGGCCCACUUUCCCCGGGAGGGCGUGUUCGUGAG | |
| | CAACGGCACCCACUGGUUCGUGACCCAGCGGAACUUCUACGAGCCC | |
| | CAGAUCAUCACCACCGACAACACCUUCGUGAGCGGCAACUGCGACG | |
| | UGGUGAUCGGCAUCGUGAACAACACCGUGUACGAUCCCCUGCAGCC | |
| | CGAGCUGGACAGCUUCAAGGAGGAGCUGGACAAGUACUUCAAGAAU | |
| | CACACCAGCCCCGACGUGGACCUGGGCGACAUCAGCGGCAUCAACG | |
| | CCAGCGUGGUGAACAUCCAGAAGGAGAUCGAUCGGCUGAACGAGGU | |
| | GGCCAAGAACCUGAACGAGAGCCUGAUCGACCUGCAGGAGCUGGGC | |
| | AAGUACGAGCAGUACAUCAAGUGGCCCUGGUACAUCUGGCUGGGCU | |
| | UCAUCGCCGGCCUGAUCGCCAUCGUGAUGGUGACCAUCAUGCUGUG | |
| | CUGCAUGACCAGCUGCUGCAGCUGCCUGAAGGGCUGUUGCAGCUGC | |
| | GGCAGCUGCUGCAAGUUCGACGAGGACGACAGCGAGCCCGUGCUGA | |
| | AGGGCGUGAAGCUGCACUACACC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGGGC | |
| Corresponding amino | MFLILLISLPTAFAVIGDLKCTSDNINDKDTGPPPISTDTVDVING | 41 |
| acid sequence | LGTYYVLDRVYLNTTLFLNGYYPTSGSTYRNMALKGSVLLSRLWFK | |
| | PPFLSDFINGIFAKVKNTKVIKDRVMYSEFPAITIGSTFVNTSYSV | |
| | VVQPRTINSTQDGDNKLQGLLEVSVCQYNMCEYPQTICHPNLGNHR | |
| | KELWHLDTGVVSCLYKRNFTYDVNADYLYFHFYQEGGTFYAYFTDT | |
| | GVVTKFLFNVYLGMALSHYYVMPLTCNSKLTLEYWVTPLTSRQYLL | |
| | AFNQDGIIFNAVDCALDPLSETKCTLKSFTVEKGIYQTNGYTVQPI | |
| | ADVYRRKPNLPNCNIEAWLNDKSVPSPLNWERKTFSNCNFNMSSLM | |
| | SFIQADSFTCNNIDAAKIYGMCFSSITIDKFAIPNGRKVDLQLGNL | |
| | GYLQSFNYRIDTTATSCQLYYNLPAANVSVSRFNPSTWNKREGFIE | |
| | DSVFKPRPAGVLTNHDVVYAQHCFKAPKNFCPCKLNGSCVGSGPGK | |
| | NNGIGTCPAGTNYLTCDNLCTPDPITFTGTYKCPQTKSLVGIGEHC | |
| | SGLAVKSDYCGGNSCTCRPQAFLGWSADSCLQGDKCNIFANFILHD | |
| | VNSGLTCSTDLQKANTDIILGVCVNYDLYGILGQGIFVEVNATYYN | |
| | SWQNLLYDSNGNLYGFRDYIINRTFMIRSCYSGGVSVITPGTNTSN | |
| | QVAVLYQDVNCTEVPVAIHADQLTPTWRVYSTGSNVFQTRAGCLIG | |
| | AEHVNNSYECDIPIGAGICASYQTQTNSPRRARSVASQSIIAYTMS | |
| | LGAENSVAYSNNSIAIPTNFTISVTTEILPVSMTKTSVDCTMYICG | |
| | DSTECSNLLLQYGSFCTQLNRALTGIAVEQDKNTQEVFAQVKQIYK | |
| | TPPIKDFGGFNFSQILPDPSKPSKRSFIEDLLENKVTLADAGFIKQ | |
| | YGDCLGDIAARDLICAQKFNGLTVLPPLLTDEMIAQYTSALLAGTI | |
| | TSGWTFGAGAALQIPFAMQMAYRFNGIGVTQNVLYENQKLIANQFN | |
| | SAIGKIQDSLSSTASALGKLQDVVNQNAQALNTLVKQLSSNFGAIS | |
| | SVLNDILSRLDPPEAEVQIDRLITGRLQSLQTYVTQQLIRAAEIRA | |
| | SANLAATKMSECVLGQSKRVDFCGKGYHLMSFPQSAPHGVVFLHVT | |
| | YVPAQEKNFTTAPAICHDGKAHFPREGVFVSNGTHWFVTQRNFYEP | |
| | QIITTDNTFVSGNCDVVIGIVNNTVYDPLQPELDSFKEELDKYFKN | |
| | HTSPDVDLGDISGINASVVNIQKEIDRLNEVAKNLNESLIDLQELG | |
| | KYEQYIKWPWYIWLGFIAGLIAIVMVTIMLCCMTSCCSCLKGCCSC | |
| | GSCCKFDEDDSEPVLKGVKLHYT | |
| PolyA tail | 100 nt | |

### Encoded Domain Antigens

Other aspects of the present disclosure provide compositions comprising an mRNA that encodes a (at least one) subdomain of the SARS-CoV-2 S1 subunit of the S protein. The subdomain may be an N-terminal domain (NTD) or a receptor binding domain (RBD) (with or without the SD1 and/or SD2). In some embodiments, an mRNA encodes a combination (e.g., a non-natural combination) of an NTD and an RBD (with or without the SD1 and/or SD2). In some embodiments, the NTD and/or RBD is linked to a transmembrane domain (with or without the SD1 and/or SD2). In some embodiments, the mRNA encodes two subdomains of the SARS-CoV-2 S1 subunit of the S protein (NTD and RBD) that have been mutated to comprise cysteine residues. Such mutations, in some embodiments, result in the formation of a disulfide bond. As an example, an mRNA may encode an NTD comprising an F43C mutation and an RBD comprising a Q563C mutation, ultimately resulting in a an NTD linked to an RBD via disulfide bond.

### N Terminal Domain (NTD) Constructs

In some embodiments, an mRNA provided herein encodes an NTD of an S1 subunit of a SARS-CoV-2 S protein. The NTD of certain betacoronaviruses elicits protective levels of antibodies. Antibodies specific to the NTD of other betacoronaviruses such as MERS act by preventing membrane fusion and viral entry (Zhou H et al. Nat Commun. 2019; 3068), providing a second mechanism of neutralization that is distinct from preventing viral attachment to ACE2. The SARS-CoV-2 NTDs encoded by an mRNA of the present disclosure may be soluble or membrane bound. A non-limiting example of a membrane bound SARS-CoV-2 NTD antigen and the mRNA encoding it is provided in Tables 6A and 6B below.

TABLE 6A
Membrane Bound NTD Antigens

| | SEQ ID NO: | |
|---|---|---|
| Name | mRNA ORF | Protein |
| SARS-CoV-2 NTD Linked to Transmembrane | 46 | 47 |
| Domain (NTD-TM) | | |

TABLE 6B
Membrane Bound NTD Antigens
SARS-CoV-2 NTD Linked to Transmembrane Domain (NTD-TM)

| SEQ ID NO: 45 consists of from 5′ end to 3′ end: | | 45 |
|---|---|---|
| 5′ UTR SEQ ID NO: 2, mRNA ORF SEQ ID | | |
| NO: 46 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 46 |
| Construct | UGAACCUGACCACCCGGACCCAGCUGCCACCAGCCUACACCAACAG | |
| (excluding | CUUCACCCGGGGCGUCUACUACCCCGACAAGGUGUUCCGGAGCAGC | |
| the stop | GUCCUGCACAGCACCCAGGACCUGUUCCUGCCCUUCUUCAGCAACG | |
| codon) | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUCUG | |
| | GCGGAGGCAGCAUCCUGGCCAUCUACAGCACCGUGGCCAGCAGCCU | |
| | GGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGG | |
| Corresponding | MFVFLVLLPLVSSQCVNLITRTQLPPAYTNSFTRGVYYPDKVERSS | 47 |
| amino | VLHSTQDLFLPFFSNVTWFHAIHVSGINGTKRFDNPVLPENDGVYF | |
| acid sequence | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDSGGGSILAIYSTVASSLVLLVSLGAISF | |
| PolyA tail | 100 nt | |

### Receptor Binding Domain (RBD) Constructs

In other embodiments, an mRNA provided herein encodes an RBD of an S1 subunit of a SARS-CoV-2 S protein. The RBD binds ACE2 receptors on host cells, which mediate virus attachment to cells. Attachment is necessary for the virus to enter cells and replicate. Thus, RBD targeted antibody responses, which block virus attachment into the cell, effectively neutralize extracellular virus particles, preventing proliferation and promoting further immune responses to other components of the neutralized virus particles. The SARS-CoV-2 RBDs encoded by an mRNA of the present disclosure may be soluble or membrane bound (e.g., linked to a transmembrane domain).

### Soluble RBD Antigens

In some embodiments, an mRNA encodes a soluble SARS-CoV-2 RBD. Dendritic cells sample soluble proteins by pinocytosis and, upon migrating to the draining lymph node, present linear peptides that comprise the sampled protein to CD4⁺ T cells. These CD4⁺ T cells provide proliferation signals to B cells that have recognized, taken up, and presented an epitope from the RBD, so administration of specifically RBD without other components of the SARS-CoV-2 spike protein expected to focus the immune response towards the epitopes present in the RBD. Non-limiting examples of soluble SARS-CoV-2 RBDs and the mRNA encoding them are provided in the Tables 7A and 7B below.

TABLE 7
Soluble RBD Antigens

SEQ ID NO:

| Name | mRNA ORF | Protein |
|---|---|---|
| SARS-CoV-2 Soluble RBD | 61 | 62 |

TABLE 7B
Soluble RBD Antigens
SARS-CoV-2 Soluble RBD

| SEQ ID NO: 60 consists of from 5′ end to 3′ end: 5′ UTR SEQ ID NO: 2, | | 60 |
|---|---|---|
| mRNA ORF SEQ ID NO: 61 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 61 |
| Construct | UGCUGGUGAACAGCCAGCCCAACAUCACCAACCUGUGCCCCUUCGG | |
| (excluding the stop | CGAGGUGUUCAACGCCACCCGGUUCGCCAGCGUGUACGCCUGGAAC | |
| codon) | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MYSMQLASCVTLTLVLLVNSQPNITNLCPFGEVENATRFASVYAWN | 62 |
| acid sequence | RKRISNCVADYSVLYNSASFSTFKCYGVSPTKLNDLCFINVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPK | |
| PolyA tail | 100 nt | |

### Membrane Bound RBD Antigens

In some embodiments, an mRNA encodes a membrane bound SARS-CoV-2 RBD. Cells expressing membrane bound RBD are expected to carry these membrane-bound antigens to the draining lymph node and promote efficient recognition of epitopes by RBD-specific B cells. Because the B cell surface contains many surface bound antibodies and the expressing cell contains many copies of the membrane bound RBD, it is expected that initial recognition of antigen by a B cell will be followed by cross-linking of B cell receptors, stimulating a strong response through an avidity effect. Non-limiting examples of membrane bound SARS-CoV-2 RBDs and the mRNA encoding them are provided in Tables 8A and 8B below.

TABLE 8A
Membrane Bound RBD Antigens

| | SEQ ID NO: | |
|---|---|---|
| Name | mRNA ORF | Protein |
| SARS-CoV-2 RBD Linked to Transmembrane | 76 | 77 |
| Domain (RBD-TM) | | |

TABLE 8B
Membrane Bound RBD Antigens
SARS-CoV-2 RBD Linked to Transmembrane Domain (RBD-TM)

| SEQ ID NO: 75 consists of from 5′ end to 3′ end: 5′ UTR SEQ ID NO: 2, | | 75 |
|---|---|---|
| mRNA ORF SEQ ID NO: 76 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 76 |
| Construct | UGCUGGUGAACAGCCAGCCCAACAUCACCAACCUGUGCCCCUUCGG | |
| (excluding the stop | CGAGGUGUUCAACGCCACCCGGUUCGCCAGCGUGUACGCCUGGAAC | |
| codon) | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MYSMQLASCVILTLVLLVNSQPNITNLCPFGEVENATRFASVYAWN | 77 |
| acid sequence | RKRISNCVADYSVLYNSASFSTFKCYGVSPTKLNDLCFINVYADSE | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKSGGGSILAIY | |
| | STVASSLVLLVSLGAISF | |
| PolyA tail | 100 nt | |

### Domain Fusion Antigens

In yet other embodiments, an mRNA provided herein encodes a SARS-CoV-2 NTD-RBD fusion protein. For example, the NTD and the RBD of a SARS-CoV-2 S1 subunit of an S protein may be linked to each other through a linker, such as a short amino acid (e.g., glycine-serine) linker to allow flexibility/hinging and space between the domains. In another embodiment, a linker comprising an antigenic epitope, e.g., a Class II universal T cell epitope such as PADRE, can be used. In some embodiments, a transmembrane region is linked to the NTD-RBD fusion, for example, through another short amino acid (e.g., glycine-serine or PADRE) linker for flexibility and to permit a reasonable distance between the membrane and the antigen. Without being bound by theory, it is thought that this membrane bound, tandem configuration presents most, if not all, known neutralizing and protective epitopes in one open reading frame. Administration of this fusion protein should then focus the immune response towards known protective epitopes and reduce the unnecessary generation of antibodies and T cells specific to non-protective epitopes. Furthermore, antibodies to different domains may neutralize virus particles through different mechanisms, such as by blocking attachment to host cells or preventing bound virus from undergoing membrane fusion and entering host cells. The broad response elicited by a fusion protein comprising different domains may thus be more evolutionarily robust, requiring multiple distinct mutations to escape vaccine-induced immunity. Non-limiting examples of SARS-CoV-2 NTD-RBD fusion proteins and the mRNA encoding them are provided in Tables 9A and 9B below.

### Linkers

A variety of linkers may be used in accordance with the present disclosure. Linkers, as provide herein, are simply amino acid sequences that artificially link together two other amino acid sequences. Linkers used herein may be cleavable or non-cleavable. Cleavable linkers allow an mRNA to be translated into a polypeptide, after which cleavage of the linker allows each individual component to be released independently. Non-cleavable linkers keep one or more protein subunits connected, allowing the whole protein to perform a function that requires close proximity of the component subunits. Non-limiting examples of such linkers include glycine-serine (GS) linkers (non-cleavable); and F2A linker, P2A linker, T2A linker, and E2Alinker (cleavable). Other links may be used herein.

In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, an mRNA encodes a fusion protein that comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or 20 amino acids. In some embodiments, a GS linker is (or is at least) 15 amino acids long (e.g., GGSGGSGGSGGSGGG (SEQ ID NO: 133)). In some embodiments, a GS linker is (or is at least) 8 amino acids long (e.g., GGGSGGGS (SEQ ID NO: 134)). In some embodiments, a GS linker is (or is at least) 7 amino acids long (e.g., GGGSGGG (SEQ ID NO: 135)). In some embodiments, a GS linker is (or is at least) 4 amino acid long (e.g., GGGS (SEQ ID NO: 136)). In some embodiments, the GS linker comprises (GGGS)n (SEQ ID NO: 136), where n is any integer from 1-5. In some embodiments, a GS linker is (or is at least) 4 amino acid long (e.g., GSGG (SEQ ID NO: 152)). In some embodiments, the GS linker comprises (GSGG)n (SEQ ID NO: 152), where n is any integer from 1-5.

In some embodiments, a linker is a glycine linker, for example having a length of (or a length of at least) 3 amino acids (e.g., GGG).

In some embodiments, a protein encoded by an mRNA vaccine includes more than one linker, which may be the same or different from each other (e.g., GGGSGGG (SEQ ID NO: 135) and GGGS (SEQ ID NO: 136) in the same S protein construct).

In some embodiments, a linker comprises mRNA encoding a pan HLA DR-binding epitope (PADRE) (e.g., AKFVAAWTLKAAA (SEQ ID NO: 148)). PADRE is an immunodominant helper CD4 T cell epitope and a potent immunogen (See, e.g., Alexander J. et al. J of Immuno. 164(3): 1625-33, incorporated herein by reference).

TABLE 9A
Domain Fusion Antigen

| | SEQ ID NO: | |
|---|---|---|
| Name | mRNA ORF | Protein |
| SARS-CoV-2 NTD-RBD Linked to | 91 | 92 |
| Transmembrane Domain (NTD-RBD-TM) | | |

TABLE 9B
Domain Fusion Antigen
SARS-CoV-2 NTD-RBD Linked to Transmembrane Domain (NTD-RBD-TM)

| SEQ ID NO: 90 consists of from 5′ end to 3′ end: 5′ UTR SEQ ID NO: 2, | | 90 |
|---|---|---|
| mRNA ORF SEQ ID NO: 91 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 91 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 92 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFINVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | stvasslvllvslgaisf | |
| PolyA tail | 100 nt | |

### Trafficking Signals

In some embodiments, an mRNA encodes a SARS-CoV-2 S protein domain (e.g., NTD, RBD, or NTD-RBD fusion) linked to a Golgi trafficking signal. Non-limiting examples of such signals include macrophage markers, such as CD86 and/or CD11b, which are highly expressed and the intracellular region may control efficient export from the Golgi apparatus to the cell surface. Other cell trafficking signals (sequences) may be used herein, for example, the VSV-G cytosolic tail (VSVGct). More efficient trafficking of encoded proteins to the cell surface is expected to increase antigen availability for B cell recognition and therefore promote the generation of antibodies to the encoded SARS-CoV-2 S protein domains. Non-limiting examples of SARS-CoV-2 antigens linked to a trafficking signal and the mRNA encoding them are provided in Tables 10A and 10B below.

TABLE 10A
Domain Fusion Antigens Linked to a Trafficking Signal

| | SEQ ID NO: | |
|---|---|---|
| Name | mRNA ORF | Protein |
| SARS-CoV-2 NTD-RBD Linked to | 94 | 95 |
| Transmembrane Domain and huCD86 | | |
| (NTD-RBD-TM-CD86) | | |
| SARS-CoV-2 NTD-RBD Linked to | 97 | 98 |
| Transmembrane Domain and huCD11B | | |
| (NTD-RBD-TM-CD11B) | | |
| SARS-CoV-2 NTD-RBD Linked to VSVGct | 109 | 110 |
| (NTD-RBD-VSVGct) | | |

TABLE 10B
Domain Fusion Antigens Linked to a Trafficking Signal
SARS-CoV-2 NTD-RBD Linked to Transmembrane Domain and huCD86 (NTD-RBD-TM-CD86)

| SEQ ID NO: 93 consists of from 5′ end to 3′ end: 5′ UTR SEQ ID NO: 2, | | 93 |
|---|---|---|
| mRNA ORF SEQ ID NO: 94 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 94 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUCAAGAAGAAGAAGCGGCCACGGAACUCCUACAAGUGCGG | |
| | CACCAACACCAUGGAGCGGGAGGAGAGCGAGCAGACCAAGAAGCGG | |
| | GAGAAGAUCCACAUUCCUGAACGGUCCGACGAAGCCCAGCGGGUGU | |
| | UCAAGAGCAGCAAGACCAGCAGCUGCGACAAGAGCGACACCUGCUU | |
| | C | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 95 |
| acid sequence | VLHSTQDLFLPFFSNVTWFHAIHVSGTNGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVFNATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | stvasslvllvslgaisfKKKKRPRNSYKCGTNTMEREESEQTKKR | |
| | EKIHIPERSDEAQRVFKSSKTSSCDKSDTCF | |
| PolyA tail | 100 nt | |

SARS-CoV-2 NTD-RBD Linked to Transmembrane
Domain and huCD11B (NTD-RBD-TM-CD11B)

| SEQ ID NO: 96 consists of from 5′ end to 3′ end: 5′ UTR SEQ ID NO: 2, | | 96 |
|---|---|---|
| mRNA ORF SEQ ID NO: 97 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 97 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUCAAGCGGCAGUACAAGGACAUGAUGAGCGAGGGAGGACC | |
| | ACCUGGCGCUGAGCCACAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 98 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | stvasslvllvslgaisfKRQYKDMMSEGGPPGAEPQ | |
| PolyA tail | 100 nt | |

SARS-CoV-2 NTD-RBD Linked to VSVGct (NTD-RBD-VSVGct)

| SEQ ID NO: 108 consists of from 5′ end to 3′ end: 5′ UTR SEQ ID NO: 2, | | 108 |
|---|---|---|
| mRNA ORF SEQ ID NO: 109 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 109 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAGCAGCAUCGCCAGC | |
| | UUCUUCUUCAUCAUCGGGCUGAUCAUCGGCCUCUUCCUGGUGCUGC | |
| | GGGUGGGCAUCCACCUGUGCAUCAAGCUGAAGCACACCAAGAAGAG | |
| | ACAGAUCUACACCGACAUCGAGAUGAACCGGCUGGGCAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 110 |
| acid sequence | VLHSTQDLFLPFFSNVTWFHAIHVSGTNGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVFNATRFASVYAWN | |
| | RKRISNCVADYSVLYNSASFSTFKCYGVSPTKLNDLCFINVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsSSIAS | |
| | FFFIIGLIIGLFLVLRVGIHLCIKLKHTKKRQIYTDIEMNRLGK | |
| PolyA tail | 100 nt | |

### Domain Fusion C-Terminal Truncations

In other embodiments, an mRNA provided herein encodes a SARS-CoV-2 NTD-RBD fusion protein in which some portion of the C-terminal domain has been truncated/deleted. In one embodiment, 13 (or at least 13) amino acids have been deleted from the C-terminal domain of the NTD-RBD fusion protein. Deletion of these amino acids is expected to increase exposure of epitopes to antibodies, thereby stimulating a more robust immune response to protective epitopes present on the NTD and RBD domains.

A non-limiting example of SARS-CoV-2 domain fusion antigen having a C-terminal truncation and the mRNA encoding it is provided in Tables 11A and 11B below.

TABLE 11A
Domain Fusion C-Terminal Truncation

| | SEQ ID NO: | |
|---|---|---|
| Name | mRNA ORF | Protein |
| SARS-CoV-2 NTD-RBD with C-terminal | 106 | 107 |
| Truncation of 13 Amino Acids (NTD-RBD-Δ13) | | |

TABLE 11B
Domain Fusion C-Terminal Truncation
SARS-CoV-2 NTD-RBD with C-terminal Truncation of 13 Amino Acids (NTD-RBD-A13)

| SEQ ID NO: 105 consists of from 5′ end to 3′ end: 5′ UTR SEQ ID NO: 2, | | 105 |
|---|---|---|
| mRNA ORF SEQ ID NO: 106 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 106 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCCUGGGCUUCAUCGCCGGCCUGAUCGCCAUCGUGAUGGUGACCA | |
| | UCAUGCUGUGCUGCAUGACCAGCUGCUGCAGCUGCCUGAAGGGCUG | |
| | UUGCAGCUGCGGCAGCUGCUGCAAGUUCGACGAGGACGAC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 107 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFINVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | SLGFIAGLIAIVMVTIMLCCMTSCCSCLKGCCSCGSCCKFDEDD | |
| PolyA tail | 100 nt | |

### Domain Extensions

SARS-CoV-2 S protein domain antigens, in some embodiments, include “extended” regions that include sequences adjacent to and/or flanking what is understood in the art to be the NTD domain or the RBD domain. The RBD_EXT series encompasses the SD1 (subdomain 1). The NTD_EXT series encompasses a C-terminal helix in the NTD. Some B cells and antibodies recognize conformational epitopes found only in properly folded, but not denatured, forms of the SARS-CoV-2 S protein NTD and RBD. Inclusion of sequences adjacent to and/or flanking the NTD and RBD domains not only can provide additional B-cell epitopes to the antigen, but may potentially result in more optimal folding of those domains and stimulate B cells with antibodies specific to epitopes that may be found on the edge of either domain. Furthermore, the inclusion of these extension sequences may thus increase the distance between the NID or RBD and the expressing cell membrane, increasing exposure of both domains to antibodies that may bind less efficiently if the expressed protein was too close to the cell surface. Finally, the inclusion of extension sequences increases the pool of peptides that could potentially be presented to CD4⁺ T cells by B cells that have recognized an NTD or RBD epitope, then processed the entire protein for antigen presentation, thereby increasing the chance that an NTD or RBD-specific B cell receives sufficient T cell help. Non-limiting example of SARS-CoV-2 domain extensions and the mRNA encoding them are provided in Tables 12A and 12B below.

TABLE 12
Domain Extensions

| | SEQ ID NO: | |
|---|---|---|
| Name | mRNA ORF | Protein |
| SARS-CoV-2 NTD DS Extended Linked to | 52 | 53 |
| Transmembrane Domain (NTD-EXT-F43C-TM) | | |
| SARS-CoV-2 NTD DS Extended Linked to | 55 | 56 |
| Transmembrane Domain (NTD-F43C-EXT-TM) | | |
| SARS-CoV-2 NTD Extended Linked to | 58 | 59 |
| Transmembrane Domain (NTD-EXT-TM) | | |
| SARS-CoV-2 RBD Extended Linked to | 85 | 86 |
| Transmembrane Domain (RBD-EXT-TM) | | |
| SARS-CoV-2 RBD DS Extended Linked to | 88 | 89 |
| Transmembrane Domain (RBD-Q563D-EXT-TM) | | |
| SARS-CoV-2 NTD-RBD Extended Linked to | 115 | 116 |
| Transmembrane Domain (NTD-RBD-EXT-TM) | | |
| SARS-CoV-2 NTD Extended-RBD Linked to | 118 | 119 |
| Transmembrane Domain (NTD-EXT-RBD-TM) | | |
| SARS-CoV-2 NTD Extended-RBD-Extended | 121 | 122 |
| Linked to Transmembrane Domain | | |
| (NTD-EXT-RBD-EXT-TM) | | |

TABLE 12B
Domain Extensions
SARS-CoV-2 NTD DS Extended Linked to Transmembrane Domain (NTD-EXT-F43C-TM)

| SEQ ID NO: 51 consists of from 5′ end to 3′ end: 5′ UTR SEQ ID NO: 2, | | 51 |
|---|---|---|
| mRNA ORF SEQ ID NO: 52 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 52 |
| Construct | UGAACCUGACCACCCGGACCCAGCUGCCACCAGCCUACACCAACAG | |
| (excluding the stop | CUUCACCCGGGGCGUCUACUACCCCGACAAGGUGUGCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUCUG | |
| | GCGGAGGCAGCAUCCUGGCCAUCUACAGCACCGUGGCCAGCAGCCU | |
| | GGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTOLPPAYTNSFTRGVYYPDKVCRSS | 53 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKOG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDsgggsilaiystvasslvllvslgaisf | |
| PolyA tail | 100 nt | |

SARS-CoV-2 NTD DS Extended Linked to Transmembrane Domain (NTD-F43C-EXT-TM)

| SEQ ID NO: 54 consists of from 5′ end to 3′ end: 5′ UTR SEQ ID NO: 2, | | 54 |
|---|---|---|
| mRNA ORF SEQ ID NO: 55 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 55 |
| Construct | UGAACCUGACCACCCGGACCCAGCUGCCACCAGCCUACACCAACAG | |
| (excluding the stop | CUUCACCCGGGGCGUCUACUACCCCGACAAGGUGUGCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CUCUGGCGGAGGCAGCAUCCUGGCCAUCUACAGCACCGUGGCCAGC | |
| | AGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVCRSS | 56 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTsgggsilaiystvas | |
| | slvllvslgaisf | |

SARS-CoV-2 NTD Extended Linked to Transmembrane Domain (NTD-EXT-TM)

| SEQ ID NO: 57 consists of from 5′ end to 3′ end: 5′ UTR SEQ ID NO: 2, | | 57 |
|---|---|---|
| mRNA ORF SEQ ID NO: 58 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 58 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CUCUGGCGGAGGCAGCAUCCUGGCCAUCUACAGCACCGUGGCCAGC | |
| | AGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 59 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTsgggsilaiystvas | |
| | slvllvslgaisf | |
| PolyA tail | 100 nt | |

SARS-CoV-2 RBD Extended Linked to Transmembrane Domain (RBD-EXT-TM)

| SEQ ID NO: 84 consists of from 5′ end to 3′ end: 5′ UTR SEQ ID NO: 2, | | 84 |
|---|---|---|
| mRNA ORF SEQ ID NO: 85 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 85 |
| Construct | UGCUGGUGAACAGCCAGCGGGUGCAGCCCACCGAGAGCAUCGUGCG | |
| (excluding the stop | GUUCCCCAACAUCACCAACCUGUGCCCCUUCGGCGAGGUGUUCAAC | |
| codon) | GCCACCCGGUUCGCCAGCGUGUACGCCUGGAACCGGAAGCGGAUCA | |
| | GCAACUGCGUGGCCGACUACAGCGUGCUGUACAACAGCGCCAGCUU | |
| | CAGCACCUUCAAGUGCUACGGCGUGAGCCCCACCAAGCUGAACGAC | |
| | CUGUGCUUCACCAACGUGUACGCCGACAGCUUCGUGAUCCGUGGCG | |
| | ACGAGGUGCGGCAGAUCGCACCCGGCCAGACAGGCAAGAUCGCCGA | |
| | CUACAACUACAAGCUGCCCGACGACUUCACCGGCUGCGUGAUCGCC | |
| | UGGAACAGCAACAACCUCGACAGCAAGGUGGGCGGCAACUACAACU | |
| | ACCUGUACCGGCUGUUCCGGAAGAGCAACCUGAAGCCCUUCGAGCG | |
| | GGACAUCAGCACCGAGAUCUACCAAGCCGGCUCCACCCCUUGCAAC | |
| | GGCGUGGAGGGCUUCAACUGCUACUUCCCUCUGCAGAGCUACGGCU | |
| | UCCAGCCCACCAACGGCGUGGGCUACCAGCCCUACCGGGUGGUGGU | |
| | GCUGAGCUUCGAGCUGCUGCACGCCCCAGCCACCGUGUGUGGCCCC | |
| | AAGAAGAGCACCAACCUGGUGAAGAACAAGUGCGUGAACUUCAACU | |
| | UCAACGGCCUUACCGGCACCGGCGUGCUGACCGAGAGCAACAAGAA | |
| | AUUCCUGCCCUUUCAGCAGUUCGGCCGGGACAUCGCCGACACCACC | |
| | GACGCUGUGCGGGAUCCCCAGACCCUGGAGAUCCUGGACAUCACCC | |
| | CUUGCAGCUCUGGCGGAGGCAGCAUCCUGGCCAUCUACAGCACCGU | |
| | GGCCAGCAGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | mysmqlascvtltlvllvnsQRVQPTESIVRFPNITNLCPFGEVEN | 86 |
| acid sequence | ATRFASVYAWNRKRISNCVADYSVLYNSASFSTFKCYGVSPTKLND | |
| | LCFTNVYADSFVIRGDEVRQIAPGQTGKIADYNYKLPDDFTGCVIA | |
| | WNSNNLDSKVGGNYNYLYRLFRKSNLKPFERDISTEIYQAGSTPCN | |
| | GVEGFNCYFPLQSYGFQPTNGVGYQPYRVVVLSFELLHAPATVCGP | |
| | KKSTNLVKNKCVNFNFNGLTGTGVLTESNKKFLPFQQFGRDIADTT | |
| | DAVRDPQTLEILDITPCSsgggsilaiystvasslvllvslgaisf | |
| PolyA tail | 100 nt | |

SARS-CoV-2 RBD DS Extended Linked to Transmembrane Domain (RBD-Q563D-EXT-TM)

| SEQ ID NO: 87 consists of from 5′ end to 3′ end: 5′ UTR SEQ ID NO: 2, | | 87 |
|---|---|---|
| mRNA ORF SEQ ID NO: 88 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 88 |
| Construct | UGCUGGUGAACAGCCAGCGGGUGCAGCCCACCGAGAGCAUCGUGCG | |
| (excluding the stop | GUUCCCCAACAUCACCAACCUGUGCCCCUUCGGCGAGGUGUUCAAC | |
| codon) | GCCACCCGGUUCGCCAGCGUGUACGCCUGGAACCGGAAGCGGAUCA | |
| | GCAACUGCGUGGCCGACUACAGCGUGCUGUACAACAGCGCCAGCUU | |
| | CAGCACCUUCAAGUGCUACGGCGUGAGCCCCACCAAGCUGAACGAC | |
| | CUGUGCUUCACCAACGUGUACGCCGACAGCUUCGUGAUCCGUGGCG | |
| | ACGAGGUGCGGCAGAUCGCACCCGGCCAGACAGGCAAGAUCGCCGA | |
| | CUACAACUACAAGCUGCCCGACGACUUCACCGGCUGCGUGAUCGCC | |
| | UGGAACAGCAACAACCUCGACAGCAAGGUGGGCGGCAACUACAACU | |
| | ACCUGUACCGGCUGUUCCGGAAGAGCAACCUGAAGCCCUUCGAGCG | |
| | GGACAUCAGCACCGAGAUCUACCAAGCCGGCUCCACCCCUUGCAAC | |
| | GGCGUGGAGGGCUUCAACUGCUACUUCCCUCUGCAGAGCUACGGCU | |
| | UCCAGCCCACCAACGGCGUGGGCUACCAGCCCUACCGGGUGGUGGU | |
| | GCUGAGCUUCGAGCUGCUGCACGCCCCAGCCACCGUGUGUGGCCCC | |
| | AAGAAGAGCACCAACCUGGUGAAGAACAAGUGCGUGAACUUCAACU | |
| | UCAACGGCCUUACCGGCACCGGCGUGCUGACCGAGAGCAACAAGAA | |
| | AUUCCUGCCCUUUUGCCAGUUCGGCCGGGACAUCGCCGACACCACC | |
| | GACGCUGUGCGGGAUCCCCAGACCCUGGAGAUCCUGGACAUCACCC | |
| | CUUGCAGCUCUGGCGGAGGCAGCAUCCUGGCCAUCUACAGCACCGU | |
| | GGCCAGCAGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | mysmqlascvtltlvllvnsQRVQPTESIVRFPNITNLCPFGEVEN | 89 |
| acid sequence | ATRFASVYAWNRKRISNCVADYSVLYNSASFSTFKCYGVSPTKIND | |
| | LCFTNVYADSFVIRGDEVRQIAPGQTGKIADYNYKLPDDFTGCVIA | |
| | WNSNNLDSKVGGNYNYLYRLFRKSNLKPFERDISTEIYQAGSTPCN | |
| | GVEGFNCYFPLQSYGFQPTNGVGYQPYRVVVLSFELLHAPATVCGP | |
| | KKSTNLVKNKCVNFNFNGLIGTGVLTESNKKFLPFCQFGRDIADTT | |
| | DAVRDPQTLEILDITPCSsgggsilaiystvasslvllvslgaisf | |
| PolyA tail | 100 nt | |

SARS-CoV-2 NTD-RBD Extended Linked to Transmembrane Domain (NTD-RBD-EXT-TM)

| SEQ ID NO: 114 consists of from 5′ end to 3′ end: 5′ UTR SEQ ID NO: 2, | | 114 |
|---|---|---|
| mRNA ORF SEQ ID NO: 115 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 115 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACAGCGGGUGCAGCCCACCGAGAGCAUCGU | |
| | GCGGUUCCCCAACAUCACCAACCUGUGCCCCUUCGGCGAGGUGUUC | |
| | AACGCCACCCGGUUCGCCAGCGUGUACGCCUGGAACCGGAAGCGGA | |
| | UCAGCAACUGCGUGGCCGACUACAGCGUGCUGUACAACAGCGCCAG | |
| | CUUCAGCACCUUCAAGUGCUACGGCGUGAGCCCCACCAAGCUGAAC | |
| | GACCUGUGCUUCACCAACGUGUACGCCGACAGCUUCGUGAUCCGUG | |
| | GCGACGAGGUGCGGCAGAUCGCACCCGGCCAGACAGGCAAGAUCGC | |
| | CGACUACAACUACAAGCUGCCCGACGACUUCACCGGCUGCGUGAUC | |
| | GCCUGGAACAGCAACAACCUCGACAGCAAGGUGGGCGGCAACUACA | |
| | ACUACCUGUACCGGCUGUUCCGGAAGAGCAACCUGAAGCCCUUCGA | |
| | GCGGGACAUCAGCACCGAGAUCUACCAAGCCGGCUCCACCCCUUGC | |
| | AACGGCGUGGAGGGCUUCAACUGCUACUUCCCUCUGCAGAGCUACG | |
| | GCUUCCAGCCCACCAACGGCGUGGGCUACCAGCCCUACCGGGUGGU | |
| | GGUGCUGAGCUUCGAGCUGCUGCACGCCCCAGCCACCGUGUGUGGC | |
| | CCCAAGAAGAGCACCAACCUGGUGAAGAACAAGUGCGUGAACUUCA | |
| | ACUUCAACGGCCUUACCGGCACCGGCGUGCUGACCGAGAGCAACAA | |
| | GAAAUUCCUGCCCUUUCAGCAGUUCGGCCGGGACAUCGCCGACACC | |
| | ACCGACGCUGUGCGGGAUCCCCAGACCCUGGAGAUCCUGGACAUCA | |
| | CCCCUUGCAGCUCUGGCGGAGGCAGCAUCCUGGCCAUCUACAGCAC | |
| | CGUGGCCAGCAGCCUGGUGCUGCUGGUGAGCCUGGGCGCCAUCAGC | |
| | UUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 116 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggQRVQPTESIVRFPNITNLCPFGEVE | |
| | NATRFASVYAWNRKRISNCVADYSVLYNSASFSTFKCYGVSPTKLN | |
| | DLCFTNVYADSFVIRGDEVRQIAPGQTGKIADYNYKLPDDFTGCVI | |
| | AWNSNNLDSKVGGNYNYLYRLFRKSNLKPFERDISTEIYQAGSTPC | |
| | NGVEGENCYFPLQSYGFQPTNGVGYQPYRVVVLSFELLHAPATVCG | |
| | PKKSTNLVKNKCVNFNFNGLTGTGVLTESNKKFLPFQQFGRDIADT | |
| | TDAVRDPQTLEILDITPCSsgggsilaiystvasslvllvslgais | |
| | f | |
| PolyA tail | 100 nt | |

SARS-CoV-2 NTD Extended-RBD Linked to Transmembrane Domain (NTD-EXT-RBD-TM)

| SEQ ID NO: 117 consists of from 5′ end to 3′ end: 5′ UTR SEQ ID NO: 2, | | 117 |
|---|---|---|
| mRNA ORF SEQ ID NO: 118 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 118 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGGAGGCGGAUCGGGAGGCGGACCCAACAUCACCAACCUGUGCCCC | |
| | UUCGGCGAGGUGUUCAACGCCACCCGGUUCGCCAGCGUGUACGCCU | |
| | GGAACCGGAAGCGGAUCAGCAACUGCGUGGCCGACUACAGCGUGCU | |
| | GUACAACAGCGCCAGCUUCAGCACCUUCAAGUGCUACGGCGUGAGC | |
| | CCCACCAAGCUGAACGACCUGUGCUUCACCAACGUGUACGCCGACA | |
| | GCUUCGUGAUCCGUGGCGACGAGGUGCGGCAGAUCGCACCCGGCCA | |
| | GACAGGCAAGAUCGCCGACUACAACUACAAGCUGCCCGACGACUUC | |
| | ACCGGCUGCGUGAUCGCCUGGAACAGCAACAACCUCGACAGCAAGG | |
| | UGGGCGGCAACUACAACUACCUGUACCGGCUGUUCCGGAAGAGCAA | |
| | CCUGAAGCCCUUCGAGCGGGACAUCAGCACCGAGAUCUACCAAGCC | |
| | GGCUCCACCCCUUGCAACGGCGUGGAGGGCUUCAACUGCUACUUCC | |
| | CUCUGCAGAGCUACGGCUUCCAGCCCACCAACGGCGUGGGCUACCA | |
| | GCCCUACCGGGUGGUGGUGCUGAGCUUCGAGCUGCUGCACGCCCCA | |
| | GCCACCGUGUGUGGCCCCAAGUCUGGCGGAGGCAGCAUCCUGGCCA | |
| | UCUACAGCACCGUGGCCAGCAGCCUGGUGCUGCUGGUGAGCCUGGG | |
| | CGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVFRSS | 119 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTgggsgggPNITNLCP | |
| | FGEVFNATRFASVYAWNRKRISNCVADYSVLYNSASFSTFKCYGVS | |
| | PTKLNDLCFTNVYADSFVIRGDEVRQIAPGQTGKIADYNYKLPDDF | |
| | TGCVIAWNSNNLDSKVGGNYNYLYRLFRKSNLKPFERDISTEIYQA | |
| | GSTPCNGVEGENCYFPLQSYGFQPTNGVGYQPYRVVVLSFELLHAP | |
| | ATVCGPKsgggsilaiystvasslvllvslgaisf | |
| PolyA tail | 100 nt | |

SARS-CoV-2 NTD Extended-RBD-Extended Linked to Transmembrane Domain
(NTD-EXT-RBD-EXT-TM)

| SEQ ID NO: 120 consists of from 5′ end to 3′ end: 5′ UTR SEQ ID NO: 2, | | 120 |
|---|---|---|
| mRNA ORF SEQ ID NO: 121 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 121 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGGAGGCGGAUCGGGAGGCGGACAGCGGGUGCAGCCCACCGAGAGC | |
| | AUCGUGCGGUUCCCCAACAUCACCAACCUGUGCCCCUUCGGCGAGG | |
| | UGUUCAACGCCACCCGGUUCGCCAGCGUGUACGCCUGGAACCGGAA | |
| | GCGGAUCAGCAACUGCGUGGCCGACUACAGCGUGCUGUACAACAGC | |
| | GCCAGCUUCAGCACCUUCAAGUGCUACGGCGUGAGCCCCACCAAGC | |
| | UGAACGACCUGUGCUUCACCAACGUGUACGCCGACAGCUUCGUGAU | |
| | CCGUGGCGACGAGGUGCGGCAGAUCGCACCCGGCCAGACAGGCAAG | |
| | AUCGCCGACUACAACUACAAGCUGCCCGACGACUUCACCGGCUGCG | |
| | UGAUCGCCUGGAACAGCAACAACCUCGACAGCAAGGUGGGCGGCAA | |
| | CUACAACUACCUGUACCGGCUGUUCCGGAAGAGCAACCUGAAGCCC | |
| | UUCGAGCGGGACAUCAGCACCGAGAUCUACCAAGCCGGCUCCACCC | |
| | CUUGCAACGGCGUGGAGGGCUUCAACUGCUACUUCCCUCUGCAGAG | |
| | CUACGGCUUCCAGCCCACCAACGGCGUGGGCUACCAGCCCUACCGG | |
| | GUGGUGGUGCUGAGCUUCGAGCUGCUGCACGCCCCAGCCACCGUGU | |
| | GUGGCCCCAAGAAGAGCACCAACCUGGUGAAGAACAAGUGCGUGAA | |
| | CUUCAACUUCAACGGCCUUACCGGCACCGGCGUGCUGACCGAGAGC | |
| | AACAAGAAAUUCCUGCCCUUUCAGCAGUUCGGCCGGGACAUCGCCG | |
| | ACACCACCGACGCUGUGCGGGAUCCCCAGACCCUGGAGAUCCUGGA | |
| | CAUCACCCCUUGCAGCUCUGGCGGAGGCAGCAUCCUGGCCAUCUAC | |
| | AGCACCGUGGCCAGCAGCCUGGUGCUGCUGGUGAGCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 122 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTgggsgggQRVQPTES | |
| | IVRFPNITNLCPFGEVENATRFASVYAWNRKRISNCVADYSVLYNS | |
| | ASFSTFKCYGVSPTKLNDLCFTNVYADSFVIRGDEVRQIAPGQTGK | |
| | IADYNYKLPDDFTGCVIAWNSNNLDSKVGGNYNYLYRLFRKSNLKP | |
| | FERDISTEIYQAGSTPCNGVEGENCYFPLQSYGFQPINGVGYQPYR | |
| | VVVLSFELLHAPATVCGPKKSTNLVKNKCVNFNFNGLTGTGVLTES | |
| | NKKFLPFQQFGRDIADTTDAVRDPQTLEILDITPCSsgggsilaiy | |
| | stvasslvllvslgaisf | |
| PolyA tail | 100 nt | |

### Domain Mixtures

The present disclosure provides, in some aspects, compositions that comprise a mixture of mRNAs encoding SARS-CoV-2 S protein subdomains. In one example, a composition comprises a mixture of an mRNA encoding an NTD (with or without SD1, SD2, and/or a transmembrane domain) and an mRNA encoding an RBD (with or without SD1, SD2, and/or a transmembrane domain). In some embodiments, a composition comprises an mRNA (e.g., SEQ ID NO: 45 or 46) encoding an NTD linked to a transmembrane domain (e.g., SEQ ID NO: 47) and an mRNA (e.g., SEQ ID NO: 75 or 76 encoding an RBD linked to a transmembrane domain (e.g., SEQ ID NO: 77).

The ratio of the concentration of one mRNA to another in a composition may be 1:1 (50:50), 1:2, 1:3, 1:4, or 1:5. In some embodiments, the ratio is 1:1. For example, a composition may comprise a 1:1 ratio of an mRNA (e.g., SEQ ID NO: 45 or 46) encoding an NTD linked to a transmembrane domain (e.g., SEQ ID NO: 47) to an mRNA (e.g., SEQ ID NO: 75 or 76 encoding an RBD linked to a transmembrane domain (e.g., SEQ ID NO: 77). In some embodiments, the ratio is 1:2. For example, a composition may comprise a 1:2 ratio of an mRNA (e.g., SEQ ID NO: 45 or 46) encoding an NTD linked to a transmembrane domain (e.g., SEQ ID NO: 47) to an mRNA (e.g., SEQ ID NO: 75 or 76) encoding an RBD linked to a transmembrane domain (e.g., SEQ ID NO: 77). Another example, a composition may comprise a 1:2 ratio of an mRNA (e.g., SEQ ID NO: 75 or 76) encoding an RBD linked to a transmembrane domain (e.g., SEQ ID NO: 77) to an mRNA (e.g., SEQ ID NO: 45 or 46) encoding an NTD linked to a transmembrane domain (e.g., SEQ ID NO: 47). Different mRNAs encoding different antigens may stimulate immune responses of varying strength (Magini D et al. PLoS ONE. 2016; 11:e0161193), and administration of an equimolar ratio of two mRNAs encoding two different antigens may result in an immune response to one but not the other (John S et al. Vaccine. 2018; 36:1689-1699). Manipulation of the ratio of co-delivered mRNAs may be useful for eliciting broad immune responses that target desired antigens with equal potency.

### Encoded Nanoparticle Antigens

The mRNA vaccines provided herein, in some embodiments, encode fusion proteins that comprise coronavirus antigens linked to a scaffold domain. In some embodiments, a scaffold domain imparts desired properties to an antigen encoded by an mRNA of the disclosure. For example, scaffold domain may improve the immunogenicity of an antigen, e.g., by altering the structure of the antigen, altering the uptake and processing of the antigen, and/or causing the antigen to bind to another molecule. In some embodiments, a scaffold domain linked to antigen facilitates self-assembly of the antigen into a viral nanoparticle or a larger protein-folded immunogen. Non-limiting examples of scaffold domains that may be used as provide herein include, ferritin domains, lumazine synthetase domains, foldon domains, and encapsulin domains. Other scaffold domains may be used.

### Ferritin

In some embodiments, a ferritin domain is used as a scaffold domain. Ferritin is a protein, the main function of which is intracellular iron storage. Ferritin is comprised of twenty-four (24) subunits, each composed of a four-alpha-helix bundle that self-assemble into a quaternary structure with octahedral symmetry (Cho K. J. et al. J Mol Biol. 2009; 390: 83-98; (Granier T. et al. J Biol Inorg Chem. 2003; 8: 105-111; and Lawson D. M. et al. Nature. 1991; 349: 541-544). Ferritin self-assembles into nanoparticles with robust thermal and chemical stability. Enclosing antigens within ferritin nanoparticles in this manner is expected to both delay degradation of the antigen and aggregate individual antigens, with each nanoparticle containing twenty-four (24) antigen subunits. Aggregation of multiple copies of the same antigen enhances both antigen uptake and migration by dendritic cells, as well as more robust CD4⁺ and CD8⁺ T cell responses (Kastenmüller K et al. J Clin Invest. 2011; 121(5):1782-96). Thus, the ferritin nanoparticle is a well-suited platform for antigen presentation and vaccine development.

An mRNA provided herein, in some embodiments, encodes an RBD linked to a ferritin domain, for example, through a glycine (e.g., GGG) linker domain. Other linkers may be used.

In other embodiments, an mRNA provided herein encodes an S1 domain of an S protein linked to a ferritin domain, for example, through a glycine (e.g., GGG) linker. As indicated elsewhere herein, other linkers may be used.

Non-limiting examples of SARS-CoV-2 antigens linked to a ferritin domain and the mRNA encoding them are provided in Tables 13A and 13B below.

TABLE 13A
Antigens Linked to a Ferritin Domain

| | SEQ ID NO: | |
|---|---|---|
| Name | mRNA ORF | Protein |
| SARS-CoV-2 S1 Subunit Linked to Ferritin | 7 | 8 |
| (S1-Ferritin) | | |
| SARS-CoV-2 RBD Linked to Ferritin | 64 | 65 |
| (RBD-Ferritin) | | |

TABLE 13B
Antigens Linked to a Ferritin Domain
SARS-CoV-2 S1 Subunit Linked to Ferritin (S1-Ferritin)

| SEQ ID NO: 6 consists of from 5′ end to 3′ end: 5′ UTR SEQ ID NO: 2, | | 6 |
|---|---|---|
| mRNA ORF SEQ ID NO: 7 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 7 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCAGGAGGAGGCAGCGGCGGCGAUAUCAUCAAG | |
| | CUUCUGAACGAGCAAGUUAACAAGGAAAUGCAGAGCAGUAAUCUCU | |
| | ACAUGAGCAUGAGCAGCUGGUGCUACACCCACUCCCUGGACGGAGC | |
| | AGGCCUCUUCCUGUUCGACCACGCAGCCGAGGAGUACGAGCACGCU | |
| | AAGAAGUUGAUCAUUUUCUUGAACGAGAACAACGUGCCCGUGCAGC | |
| | UAACGUCAAUCAGCGCACCUGAGCACAAGUUCGAGGGCCUGACCCA | |
| | GAUCUUCCAGAAGGCCUACGAACACGAACAGCACAUCUCCGAGAGC | |
| | AUCAACAAUAUUGUGGAUCACGCUAUCAAGUCCAAGGACCACGCUA | |
| | CCUUCAACUUCCUGCAGUGGUACGUGGCCGAGCAACAUGAGGAGGA | |
| | GGUGCUGUUCAAGGACAUCCUGGACAAGAUCGAGCUGAUCGGUAAU | |
| | GAGAAUCACGGCCUGUACCUGGCCGACCAGUACGUGAAGGGCAUCG | |
| | CCAAGAGCCGGAAGUCAGGCUCA | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 8 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNSgggSGGDIIK | |
| | LLNEQVNKEMQSSNLYMSMSSWCYTHSLDGAGLFLFDHAAEEYEHA | |
| | KKLIIFLNENNVPVQLTSISAPEHKFEGLTQIFQKAYEHEQHISES | |
| | INNIVDHAIKSKDHATFNFLQWYVAEQHEEEVLFKDILDKIELIGN | |
| | ENHGLYLADQYVKGIAKSRKSGS | |
| PolyA tail | 100 nt | |

SARS-CoV-2 RBD Linked to Ferritin (RBD-Ferritin)

| SEQ ID NO: 63 consists of from 5′ end to 3′ end: 5′ UTR SEQ ID NO: 2, | | 63 |
|---|---|---|
| mRNA ORF SEQ ID NO: 64 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 64 |
| Construct | UGCUGGUGAACAGCCAGCCCAACAUCACCAACCUGUGCCCCUUCGG | |
| (excluding the stop | CGAGGUGUUCAACGCCACCCGGUUCGCCAGCGUGUACGCCUGGAAC | |
| codon) | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGGGAGGAGGCAGCGGCGGCGAUAUCAUCAAG | |
| | CUUCUGAACGAGCAAGUUAACAAGGAAAUGCAGAGCAGUAAUCUCU | |
| | ACAUGAGCAUGAGCAGCUGGUGCUACACCCACUCCCUGGACGGAGC | |
| | AGGCCUCUUCCUGUUCGACCACGCAGCCGAGGAGUACGAGCACGCU | |
| | AAGAAGUUGAUCAUUUUCUUGAACGAGAACAACGUGCCCGUGCAGC | |
| | UAACGUCAAUCAGCGCACCUGAGCACAAGUUCGAGGGCCUGACCCA | |
| | GAUCUUCCAGAAGGCCUACGAACACGAACAGCACAUCUCCGAGAGC | |
| | AUCAACAAUAUUGUGGAUCACGCUAUCAAGUCCAAGGACCACGCUA | |
| | CCUUCAACUUCCUGCAGUGGUACGUGGCCGAGCAACAUGAGGAGGA | |
| | GGUGCUGUUCAAGGACAUCCUGGACAAGAUCGAGCUGAUCGGUAAU | |
| | GAGAAUCACGGCCUGUACCUGGCCGACCAGUACGUGAAGGGCAUCG | |
| | CCAAGAGCCGGAAGUCAGGCUCA | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MYSMQLASCVTLTLVLLVNSQPNITNLCPFGEVENATRFASVYAWN | 65 |
| acid sequence | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKGGGSGGDIIK | |
| | LLNEQVNKEMQSSNLYMSMSSWCYTHSLDGAGLFLFDHAAEEYEHA | |
| | KKLIIFLNENNVPVQLTSISAPEHKFEGLTQIFQKAYEHEQHISES | |
| | INNIVDHAIKSKDHATFNFLQWYVAEQHEEEVLFKDILDKIELIGN | |
| | ENHGLYLADQYVKGIAKSRKSGS | |
| PolyA tail | 100 nt | |

### Lumazine Synthetase

In some embodiments, a lumazine synthetase domain is used as a scaffold domain. Lumazine synthetase is an enzyme responsible for the penultimate catalytic step in the biosynthesis of riboflavin in a variety of organisms, including archaea, bacteria, fungi, plants, and eubacteria. Lumazine synthetase is composed of homooligomers, which vary in size and subunit number, including pentamers, decamers, and icosahedral sixty-mers, depending on its species of origin. The lumazine synthetase monomer is 150 amino acids long and includes beta-sheets with flanking, tandem alpha-helices. Different quaternary structures have been reported for lumazine synthetase, illustrating its morphological versatility: from homopentamers up to symmetrical assemblies of twelve (12) pentamers forming capsids of 150 Å diameter. Presentation of antigens on the surface of lumazine synthetase results in a high local concentration of antigens displayed in an ordered array. Such repetitive structures enable the cross-linking of B-cell receptors and result in strong immune responses through an avidity effect.

An mRNA provided herein, in some embodiments, encodes an RBD linked to a lumazine synthetase domain, for example, through a glycine-serine (e.g., GGS). Other linkers may be used.

In other embodiments, an mRNA provided herein encodes an S1 domain of an S protein linked to a lumazine synthetase domain, for example, through a glycine-serine (e.g., GGS) linker. As indicated elsewhere herein, other linkers may be used.

Non-limiting examples of SARS-CoV-2 antigens linked to a foldon domain and the mRNA encoding them are provided in Tables 14A and 14B below.

TABLE 14A
Antigens Linked to a Lumazine Synthetase Domain

| | |
|---|---|
| Name | SEQ ID NO: |

| | | |
|---|---|---|
| SARS-CoV-2 Soluble S1 Linked to Lumazine | 10 | 11 |
| Synthetase C-terminus (LS-S1) | | |
| SARS-CoV-2 Soluble S1 Linked to Lumazine | 13 | 14 |
| Synthetase N-Terminus (S1-LS) | | |
| SARS-CoV-2 RBD Linked to Lumazine | 67 | 68 |
| Synthetase C Terminus (LS-RBD) | | |
| SARS-CoV-2 RBD Linked to Lumazine | 70 | 71 |
| Synthetase N Terminus (RBD-LS) | | |

TABLE 14B
Antigens Linked to a Lumazine Synthetase Domain
SARS-CoV-2 Soluble S1 Linked to Lumazine Synthetase C-terminus (LS-S1)

| SEQ ID NO: 9 consists of from 5′ end to 3′ end: 5′ UTR SEQ ID NO: 2, | | 9 |
|---|---|---|
| mRNA ORF SEQ ID NO: 10 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGGGCAUCCUGCCCAGCCCUGGCAUGCCCGCUCUGCUGAGCCUGG | 10 |
| Construct | UGAGCCUGCUGAGCGUGCUGCUGAUGGGCUGCGUGGCUGAGACCGG | |
| (excluding | CAUGCAGAUCUACGAGGGCAAGCUGACCGCAGAGGGCCUGCGGUUC | |
| the stop | GGCAUCGUGGCCAGCCGCGCCAACCACGCUCUGGUGGACCGGCUUG | |
| codon) | UGGAGGGCGCUAUCGACGCCAUCGUGAGACACGGCGGCCGGGAAGA | |
| | GGACAUCACCCUGGUGCGGGUGUGCGGCAGCUGGGAGAUUCCCGUC | |
| | GCCGCCGGAGAACUGGCCCGGAAGGAGGACAUCGACGCCGUGAUCG | |
| | CCAUCGGCGUGCUGUGCAGAGGCGCCACGCCCAGCUUCGACUACAU | |
| | CGCCAGCGAGGUGAGCAAGGGCCUGGCCGACCUGAGCCUGGAGCUG | |
| | CGGAAGCCCAUCACCUUCGGCGUGAUCACCGCCGACACCCUGGAGC | |
| | AGGCCAUCGAGGCCGCAGGCACCUGCCACGGCAACAAGGGCUGGGA | |
| | AGCCGCCCUGUGCGCCAUCGAGAUGGCCAACCUGUUCAAGAGCCUG | |
| | CGGGGCGGAAGUGGAGGCUCUGGUGGCAGCGGAGGAUCUGGCGGCG | |
| | GCACCACCCGGACCCAGCUGCCACCAGCCUACACCAACAGCUUCAC | |
| | CCGGGGCGUCUACUACCCCGACAAGGUGUUCCGGAGCAGCGUCCUG | |
| | CACAGCACCCAGGACCUGUUCCUGCCCUUCUUCAGCAACGUGACCU | |
| | GGUUCCACGCCAUCCACGUGAGCGGCACCAACGGCACCAAGCGGUU | |
| | CGACAACCCCGUGCUGCCCUUCAACGACGGCGUGUACUUCGCCAGC | |
| | ACCGAGAAGAGCAACAUCAUCCGGGGCUGGAUCUUCGGCACCACCC | |
| | UGGACAGCAAGACCCAGAGCCUGCUGAUCGUGAAUAACGCCACCAA | |
| | CGUGGUGAUCAAGGUGUGCGAGUUCCAGUUCUGCAACGACCCCUUC | |
| | CUGGGCGUGUACUACCACAAGAACAACAAGAGCUGGAUGGAGAGCG | |
| | AGUUCCGGGUGUACAGCAGCGCCAACAACUGCACCUUCGAGUACGU | |
| | GAGCCAGCCCUUCCUGAUGGACCUGGAGGGCAAGCAGGGCAACUUC | |
| | AAGAACCUGCGGGAGUUCGUGUUCAAGAACAUCGACGGCUACUUCA | |
| | AGAUCUACAGCAAGCACACCCCAAUCAACCUGGUGCGGGAUCUGCC | |
| | CCAGGGCUUCUCAGCCCUGGAGCCCCUGGUGGACCUGCCCAUCGGC | |
| | AUCAACAUCACCCGGUUCCAGACCCUGCUGGCCCUGCACCGGAGCU | |
| | ACCUGACCCCAGGCGACAGCAGCAGCGGGUGGACAGCAGGCGCGGC | |
| | UGCUUACUACGUGGGCUACCUGCAGCCCCGGACCUUCCUGCUGAAG | |
| | UACAACGAGAACGGCACCAUCACCGACGCCGUGGACUGCGCCCUGG | |
| | ACCCUCUGAGCGAGACCAAGUGCACCCUGAAGAGCUUCACCGUGGA | |
| | GAAGGGCAUCUACCAGACCAGCAACUUCCGGGUGCAGCCCACCGAG | |
| | AGCAUCGUGCGGUUCCCCAACAUCACCAACCUGUGCCCCUUCGGCG | |
| | AGGUGUUCAACGCCACCCGGUUCGCCAGCGUGUACGCCUGGAACCG | |
| | GAAGCGGAUCAGCAACUGCGUGGCCGACUACAGCGUGCUGUACAAC | |
| | AGCGCCAGCUUCAGCACCUUCAAGUGCUACGGCGUGAGCCCCACCA | |
| | AGCUGAACGACCUGUGCUUCACCAACGUGUACGCCGACAGCUUCGU | |
| | GAUCCGUGGCGACGAGGUGCGGCAGAUCGCACCCGGCCAGACAGGC | |
| | AAGAUCGCCGACUACAACUACAAGCUGCCCGACGACUUCACCGGCU | |
| | GCGUGAUCGCCUGGAACAGCAACAACCUCGACAGCAAGGUGGGCGG | |
| | CAACUACAACUACCUGUACCGGCUGUUCCGGAAGAGCAACCUGAAG | |
| | CCCUUCGAGCGGGACAUCAGCACCGAGAUCUACCAAGCCGGCUCCA | |
| | CCCCUUGCAACGGCGUGGAGGGCUUCAACUGCUACUUCCCUCUGCA | |
| | GAGCUACGGCUUCCAGCCCACCAACGGCGUGGGCUACCAGCCCUAC | |
| | CGGGUGGUGGUGCUGAGCUUCGAGCUGCUGCACGCCCCAGCCACCG | |
| | UGUGUGGCCCCAAGAAGAGCACCAACCUGGUGAAGAACAAGUGCGU | |
| | GAACUUCAACUUCAACGGCCUUACCGGCACCGGCGUGCUGACCGAG | |
| | AGCAACAAGAAAUUCCUGCCCUUUCAGCAGUUCGGCCGGGACAUCG | |
| | CCGACACCACCGACGCUGUGCGGGAUCCCCAGACCCUGGAGAUCCU | |
| | GGACAUCACCCCUUGCAGCUUCGGCGGCGUGAGCGUGAUCACCCCA | |
| | GGCACCAACACCAGCAACCAGGUGGCCGUGCUGUACCAGGACGUGA | |
| | ACUGCACCGAGGUGCCCGUGGCCAUCCACGCCGACCAGCUGACACC | |
| | CACCUGGCGGGUCUACAGCACCGGCAGCAACGUGUUCCAGACCCGG | |
| | GCCGGUUGCCUGAUCGGCGCCGAGCACGUGAACAACAGCUACGAGU | |
| | GCGACAUCCCCAUCGGCGCCGGCAUCUGUGCCAGCUACCAGACCCA | |
| | GACCAAUUCA | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding | MGILPSPGMPALLSLVSLLSVLLMGCVAETGMQIYEGKLTAEGLRF | 11 |
| amino | GIVASRANHALVDRLVEGAIDAIVRHGGREEDITLVRVCGSWEIPV | |
| acid sequence | AAGELARKEDIDAVIAIGVLCRGATPSFDYIASEVSKGLADLSLEL | |
| | RKPITFGVITADTLEQAIEAAGTCHGNKGWEAALCAIEMANLFKSL | |
| | RGGSGGSGGSGGSGGGTTRTQLPPAYTNSFTRGVYYPDKVFRSSVL | |
| | HSTQDLFLPFFSNVTWFHAIHVSGTNGTKRFDNPVLPENDGVYFAS | |
| | TEKSNIIRGWIFGTTLDSKTQSLLIVNNATNVVIKVCEFQFCNDPF | |
| | LGVYYHKNNKSWMESEFRVYSSANNCTFEYVSQPFLMDLEGKQGNF | |
| | KNLREFVFKNIDGYFKIYSKHTPINLVRDLPQGFSALEPLVDLPIG | |
| | INITRFQTLLALHRSYLTPGDSSSGWTAGAAAYYVGYLQPRTELLK | |
| | YNENGTITDAVDCALDPLSETKCTLKSFTVEKGIYQTSNERVQPTE | |
| | SIVRFPNITNLCPFGEVENATRFASVYAWNRKRISNCVADYSVLYN | |
| | SASFSTFKCYGVSPTKLNDLCFTNVYADSFVIRGDEVRQIAPGQTG | |
| | KIADYNYKLPDDFTGCVIAWNSNNLDSKVGGNYNYLYRLFRKSNLK | |
| | PFERDISTEIYQAGSTPCNGVEGENCYFPLQSYGFQPINGVGYQPY | |
| | RVVVLSFELLHAPATVCGPKKSTNLVKNKCVNFNFNGLIGTGVLTE | |
| | SNKKFLPFQQFGRDIADTTDAVRDPQTLEILDITPCSFGGVSVITP | |
| | GTNTSNQVAVLYQDVNCTEVPVAIHADQLTPTWRVYSTGSNVFQTR | |
| | AGCLIGAEHVNNSYECDIPIGAGICASYQTQTNS | |
| PolyA tail | 100 nt | |

SARS-CoV-2 Soluble S1 Linked to Lumazine Synthetase N-Terminus (S1-LS)

| SEQ ID NO: 12 consists of from 5′ end to 3′ end: 5′ UTR SEQ ID NO: 2, | | 12 |
|---|---|---|
| mRNA ORF SEQ ID NO: 13 and 3′ UTR SEQ ID NO: 4. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 13 |
| Construct | UGAACCUGACCACCCGGACCCAGCUGCCACCAGCCUACACCAACAG | |
| (excluding | CUUCACCCGGGGCGUCUACUACCCCGACAAGGUGUUCCGGAGCAGC | |
| the stop | GUCCUGCACAGCACCCAGGACCUGUUCCUGCCCUUCUUCAGCAACG | |
| codon) | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCAGGAGGAGGCUCCGGAGGCGGUAGCGCUGAG | |
| | ACCGGCAUGCAGAUCUACGAGGGCAAGCUGACCGCAGAGGGCCUGC | |
| | GGUUCGGCAUCGUGGCCAGCCGCGCCAACCACGCUCUGGUGGACCG | |
| | GCUUGUGGAGGGCGCUAUCGACGCCAUCGUGAGACACGGCGGCCGG | |
| | GAAGAGGACAUCACCCUGGUGCGGGUGUGCGGCAGCUGGGAGAUUC | |
| | CCGUCGCCGCCGGAGAACUGGCCCGGAAGGAGGACAUCGACGCCGU | |
| | GAUCGCCAUCGGCGUGCUGUGCAGAGGCGCCACGCCCAGCUUCGAC | |
| | UACAUCGCCAGCGAGGUGAGCAAGGGCCUGGCCGACCUGAGCCUGG | |
| | AGCUGCGGAAGCCCAUCACCUUCGGCGUGAUCACCGCCGACACCCU | |
| | GGAGCAGGCCAUCGAGGCCGCAGGCACCUGCCACGGCAACAAGGGC | |
| | UGGGAAGCCGCCCUGUGCGCCAUCGAGAUGGCCAACCUGUUCAAGA | |
| | GCCUGCGG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding | MFVFLVLLPLVSSQCVNLTTRTQLPPAYTNSFTRGVYYPDKVERSS | 14 |
| amino | VLHSTQDLFLPFFSNVTWFHAIHVSGTNGTKRFDNPVLPFNDGVYF | |
| acid sequence | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNSgggsgggsAE | |
| | TGMQIYEGKLTAEGLRFGIVASRANHALVDRLVEGAIDAIVRHGGR | |
| | EEDITLVRVCGSWEIPVAAGELARKEDIDAVIAIGVLCRGATPSFD | |
| | YIASEVSKGLADLSLELRKPITFGVITADTLEQAIEAAGTCHGNKG | |
| | WEAALCAIEMANLFKSLR | |

SARS-CoV-2 RBD Linked to Lumazine Synthetase C Terminus (LS-RBD)

| SEQ ID NO: 66 consists of from 5′ end to 3′ end: 5′ UTR SEQ ID NO: 2, | | 66 |
|---|---|---|
| mRNA ORF SEQ ID NO: 67 and 3′ UTR SEQ ID NO: 4. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGGGCAUCCUGCCCAGCCCUGGCAUGCCCGCUCUGCUGAGCCUGG | 67 |
| Construct | UGAGCCUGCUGAGCGUGCUGCUGAUGGGCUGCGUGGCUGAGACCGG | |
| (excluding | CAUGCAGAUCUACGAGGGCAAGCUGACCGCAGAGGGCCUGCGGUUC | |
| the stop | GGCAUCGUGGCCAGCCGCGCCAACCACGCUCUGGUGGACCGGCUUG | |
| codon) | UGGAGGGCGCUAUCGACGCCAUCGUGAGACACGGCGGCCGGGAAGA | |
| | GGACAUCACCCUGGUGCGGGUGUGCGGCAGCUGGGAGAUUCCCGUC | |
| | GCCGCCGGAGAACUGGCCCGGAAGGAGGACAUCGACGCCGUGAUCG | |
| | CCAUCGGCGUGCUGUGCAGAGGCGCCACGCCCAGCUUCGACUACAU | |
| | CGCCAGCGAGGUGAGCAAGGGCCUGGCCGACCUGAGCCUGGAGCUG | |
| | CGGAAGCCCAUCACCUUCGGCGUGAUCACCGCCGACACCCUGGAGC | |
| | AGGCCAUCGAGGCCGCAGGCACCUGCCACGGCAACAAGGGCUGGGA | |
| | AGCCGCCCUGUGCGCCAUCGAGAUGGCCAACCUGUUCAAGAGCCUG | |
| | CGGGGCGGAAGUGGAGGCUCUGGUGGCAGCGGAGGAUCUGGCGGCG | |
| | GCCAGCCCAACAUCACCAACCUGUGCCCCUUCGGCGAGGUGUUCAA | |
| | CGCCACCCGGUUCGCCAGCGUGUACGCCUGGAACCGGAAGCGGAUC | |
| | AGCAACUGCGUGGCCGACUACAGCGUGCUGUACAACAGCGCCAGCU | |
| | UCAGCACCUUCAAGUGCUACGGCGUGAGCCCCACCAAGCUGAACGA | |
| | CCUGUGCUUCACCAACGUGUACGCCGACAGCUUCGUGAUCCGUGGC | |
| | GACGAGGUGCGGCAGAUCGCACCCGGCCAGACAGGCAAGAUCGCCG | |
| | ACUACAACUACAAGCUGCCCGACGACUUCACCGGCUGCGUGAUCGC | |
| | CUGGAACAGCAACAACCUCGACAGCAAGGUGGGCGGCAACUACAAC | |
| | UACCUGUACCGGCUGUUCCGGAAGAGCAACCUGAAGCCCUUCGAGC | |
| | GGGACAUCAGCACCGAGAUCUACCAAGCCGGCUCCACCCCUUGCAA | |
| | CGGCGUGGAGGGCUUCAACUGCUACUUCCCUCUGCAGAGCUACGGC | |
| | UUCCAGCCCACCAACGGCGUGGGCUACCAGCCCUACCGGGUGGUGG | |
| | UGCUGAGCUUCGAGCUGCUGCACGCCCCAGCCACCGUGUGUGGCCC | |
| | CAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding | MGILPSPGMPALLSLVSLLSVLLMGCVAETGMQIYEGKLTAEGLRF | 68 |
| amino acid | GIVASRANHALVDRLVEGAIDAIVRHGGREEDITLVRVCGSWEIPV | |
| sequence | AAGELARKEDIDAVIAIGVLCRGATPSFDYIASEVSKGLADLSLEL | |
| | RKPITFGVITADTLEQAIEAAGTCHGNKGWEAALCAIEMANLFKSL | |
| | RGGSGGSGGSGGSGGGQPNITNLCPFGEVENATRFASVYAWNRKRI | |
| | SNCVADYSVLYNSASFSTFKCYGVSPTKLNDLCFINVYADSFVIRG | |
| | DEVRQIAPGQTGKIADYNYKLPDDFTGCVIAWNSNNLDSKVGGNYN | |
| | YLYRLFRKSNLKPFERDISTEIYQAGSTPCNGVEGENCYFPLQSYG | |
| | FQPTNGVGYQPYRVVVLSFELLHAPATVCGPK | |
| PolyA tail | 100 nt | |

SARS-CoV-2 RBD Linked to Lumazine Synthetase N Terminus (RBD-LS)

| SEQ ID NO: 69 consists of from 5′ end to 3′ end: 5′ UTR SEQ ID NO: 2, | | 69 |
|---|---|---|
| mRNA ORF SEQ ID NO: 70 and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 70 |
| Construct | UGCUGGUGAACAGCCAGCCCAACAUCACCAACCUGUGCCCCUUCGG | |
| (excluding | CGAGGUGUUCAACGCCACCCGGUUCGCCAGCGUGUACGCCUGGAAC | |
| the stop | CGGAAGCGGAUCAGCAACUGCGUGGCCGACUACAGCGUGCUGUACA | |
| codon) | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGGGAGGAGGCUCCGGAGGCGGUAGCGCUGAG | |
| | ACCGGCAUGCAGAUCUACGAGGGCAAGCUGACCGCAGAGGGCCUGC | |
| | GGUUCGGCAUCGUGGCCAGCCGCGCCAACCACGCUCUGGUGGACCG | |
| | GCUUGUGGAGGGCGCUAUCGACGCCAUCGUGAGACACGGCGGCCGG | |
| | GAAGAGGACAUCACCCUGGUGCGGGUGUGCGGCAGCUGGGAGAUUC | |
| | CCGUCGCCGCCGGAGAACUGGCCCGGAAGGAGGACAUCGACGCCGU | |
| | GAUCGCCAUCGGCGUGCUGUGCAGAGGCGCCACGCCCAGCUUCGAC | |
| | UACAUCGCCAGCGAGGUGAGCAAGGGCCUGGCCGACCUGAGCCUGG | |
| | AGCUGCGGAAGCCCAUCACCUUCGGCGUGAUCACCGCCGACACCCU | |
| | GGAGCAGGCCAUCGAGGCCGCAGGCACCUGCCACGGCAACAAGGGC | |
| | UGGGAAGCCGCCCUGUGCGCCAUCGAGAUGGCCAACCUGUUCAAGA | |
| | GCCUGCGG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MYSMQLASCVTLTLVLLVNSQPNITNLCPFGEVENATRFASVYAWN | 71 |
| acid sequence | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKgggsgggsAE | |
| | TGMQIYEGKLTAEGLRFGIVASRANHALVDRLVEGAIDAIVRHGGR | |
| | EEDITLVRVCGSWEIPVAAGELARKEDIDAVIAIGVLCRGATPSFD | |
| | YIASEVSKGLADLSLELRKPITFGVITADTLEQAIEAAGTCHGNKG | |
| | WEAALCAIEMANLFKSLR | |
| PolyA tail | 100 nt | |

### Foldon

In some embodiments, a foldon domain is used as a scaffold domain. The C-terminal domain of T4 fibritin (foldon) is obligatory for the formation of the fibritin trimer structure and can be used as an artificial trimerization domain (see, e.g., Meier S. et al. Journal of Molecular Biology 2004 Dec. 3; 344(4): 1051-1069; Tao Y et al. Structure 1997 Jun. 15; 5(6):789-98). When fused to the S protein ectodomain, a foldon domain promotes correct trimerization of the S protein, thus avoiding misfolding of the protein. Such a process resulting in production of the prefusion conformation of the S protein results in increased expression, conformational homogeneity, and elicitation of potent neutralizing antibody responses.

Without being bound by theory, it is thought that this configuration would result in the foldon being largely immunogenically silent on the intracellular region of the protein. Non-limiting examples of SARS-CoV-2 antigens linked to a foldon domain and the mRNA encoding them are provided in Tables 15A and 15B below.

TABLE 15A
Antigens Linked to a Foldon Domain

| | SEQ ID NO: | |
|---|---|---|
| Name | mRNA ORF | Protein |
| SARS-CoV-2 NTD Linked to Foldon Domain | 43 | 44 |
| SARS-CoV-2 NTD Linked to Transmembrane | 49 | 50 |
| Domain and Foldon Domain | | |
| SARS-CoV-2 RBD Linked to Foldon Domain | 73 | 74 |
| SARS-CoV-2 RBD Linked to Foldon Domain | 79 | 80 |
| and Transmembrane Domain (RBD-FD-TM) | | |
| SARS-CoV-2 RBD Linked to Transmembrane | 82 | 83 |
| Domain and Foldon Domain (RBD-TM-FD) | | |
| SARS-CoV-2 NTD-RBD Linked to Foldon | 100 | 101 |
| Domain and Transmembrane Domain | | |
| (NTD-RBD-FD-TM) | | |
| SARS-CoV-2 NTD-RBD Linked to | 103 | 104 |
| Transmembrane Domain and Foldon Domain | | |
| (NTD-RBD-TM-FD) | | |
| SARS-CoV-2 NTD-RBD Linked to Foldon | 112 | 113 |
| Domain | | |

TABLE 15B
Antigens Linked to a Foldon Domain
SARS-CoV-2 NTD Linked to Foldon Domain

| | |
|---|---|
| SEQ ID NO: 42 consists of from 5′ end to 3′ end: 5′ UTR SEQ ID NO: 2, | 42 |
| mRNA ORF SEQ ID NO: 43 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 43 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUCUG | |
| | GCGGAGGCGGCAGCGCCAUCGGCGGCUACAUCCCCGAGGCCCCUAG | |
| | AGACGGCCAGGCCUACGUGCGGAAGGACGGCGAGUGGGUGCUGCUG | |
| | AGCACCUUCCUGGGC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGGGC | |
| Corresponding amino | MFVFLVLLPLVSSQCVNLITRTOLPPAYTNSFTRGVYYPDKVFRSS | 44 |
| acid sequence | VLHSTQDLFLPFFSNVTWFHAIHVSGINGTKRFDNPVLPENDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKOG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDsggggSAIGGYIPEAPRDGQAYVRKDGEWVLL | |
| | STFLG | |
| PolyA tail | 100 nt | |

SARS-CoV-2 NTD Linked to Transmembrane Domain and Foldon Domain

| | |
|---|---|
| SEQ ID NO: 48 consists of from 5′ end to 3′ end: 5′ UTR SEQ ID NO: 2, | 48 |
| mRNA ORF SEQ ID NO: 49 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 49 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUCUG | |
| | GCGGAGGCAGCAUCCUGGCCAUCUACAGCACCGUGGCCAGCAGCCU | |
| | GGUGCUGCUGGUGAGCCUGGGCGCCAUCAGCUUCGGCGGAGGCAGC | |
| | GCCAUCGGCGGCUACAUCCCCGAGGCCCCUAGAGACGGCCAGGCCU | |
| | ACGUGCGGAAGGACGGCGAGUGGGUGCUGCUGAGCACCUUCCUGGG | |
| | CAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGGGC | |
| Corresponding amino | MFVFLVLLPLVSSQCVNLITRTQLPPAYTNSFTRGVYYPDKVFRSS | 50 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDsgggsilaiystvasslvllvslgaisfgggS | |
| | AIGGYIPEAPRDGQAYVRKDGEWVLLSTFLGK | |
| PolyA tail | 100 nt | |

SARS-CoV-2 RBD Linked to Foldon Domain

| | |
|---|---|
| SEQ ID NO: 72 consists of from 5′ end to 3′ end: 5′ UTR SEQ ID NO: 2, | 72 |
| mRNA ORF SEQ ID NO: 73 and 3′ UTR SEQ ID NO: 4. | |

| | | |
|---|---|---|
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 73 |
| Construct | UGCUGGUGAACAGCCAGCCCAACAUCACCAACCUGUGCCCCUUCGG | |
| | CGAGGUGUUCAACGCCACCCGGUUCGCCAGCGUGUACGCCUGGAAC | |
| (excluding | CGGAAGCGGAUCAGCAACUGCGUGGCCGACUACAGCGUGCUGUACA | |
| the stop | ACAGCGCCAGCUUCAGCACCUUCAAGUGCUACGGCGUGAGCCCCAC | |
| codon) | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCGGCAGCGCCAUCGGCGGC | |
| | UACAUCCCCGAGGCCCCUAGAGACGGCCAGGCCUACGUGCGGAAGG | |
| | ACGGCGAGUGGGUGCUGCUGAGCACCUUCCUGGGC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | mysmqlascvtltlvllvnsQPNITNLCPFGEVENATRFASVYAWN | 74 |
| acid sequence | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsggggSAIGG | |
| | YIPEAPRDGQAYVRKDGEWVLLSTFLG | |
| PolyA tail | 100 nt | |

SARS-CoV-2 RBD Linked to Foldon Domain and Transmembrane Domain (RBD-FD-TM)

| | |
|---|---|
| SEQ ID NO: 78 consists of from 5′ end to 3′ end: 5′ UTR SEQ ID NO: 2, | 78 |
| mRNA ORF SEQ ID NO: 79 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 79 |
| Construct | UGCUGGUGAACAGCCAGCCCAACAUCACCAACCUGUGCCCCUUCGG | |
| (excluding the stop | CGAGGUGUUCAACGCCACCCGGUUCGCCAGCGUGUACGCCUGGAAC | |
| codon) | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCGGCAGCGCCAUCGGCGGC | |
| | UACAUCCCCGAGGCCCCUAGAGACGGCCAGGCCUACGUGCGGAAGG | |
| | ACGGCGAGUGGGUGCUGCUGAGCACCUUCCUGGGCGGAGGCAGCAU | |
| | CCUGGCCAUCUACAGCACCGUGGCCAGCAGCCUGGUGCUGCUGGUG | |
| | AGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | mysmqlascvtltlvllvnsQPNITNLCPFGEVENATRFASVYAWN | 80 |
| acid sequence | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsggggSAIGG | |
| | YIPEAPRDGQAYVRKDGEWVLLSTFLGggsilaiystvasslvllv | |
| | slgaisf | |
| PolyA tail | 100 nt | |

SARS-CoV-2 RBD Linked to Transmembrane Domain and Foldon Domain (RBD-TM-FD)

| | |
|---|---|
| SEQ ID NO: 81 consists of from 5′ end to 3′ end: 5′ UTR SEQ ID NO: 2, | 81 |
| mRNA ORF SEQ ID NO: 82 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUACAGCAUGCAGCUGGCUAGCUGCGUGACCCUGACCCUGGUGC | 82 |
| Construct | UGCUGGUGAACAGCCAGCCCAACAUCACCAACCUGUGCCCCUUCGG | |
| (excluding the stop | CGAGGUGUUCAACGCCACCCGGUUCGCCAGCGUGUACGCCUGGAAC | |
| codon) | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUCGGCGGAGGCAGCGCCAUCGGCGGCUACAUCCCCGAGGC | |
| | CCCUAGAGACGGCCAGGCCUACGUGCGGAAGGACGGCGAGUGGGUG | |
| | CUGCUGAGCACCUUCCUGGGCAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGGGC | |
| Corresponding amino | mysmqlascvtltlvllvnsQPNITNLCPFGEVENATRFASVYAWN | 83 |
| acid sequence | RKRISNCVADYSVLYNSASFSTFKCYGVSPTKLNDLCFINVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | stvasslvllvslgaisfgggSAIGGYIPEAPRDGQAYVRKDGEWV | |
| | LLSTFLGk | |
| PolyA tail | 100 nt | |

SARS-CoV-2 NTD-RBD Linked to Foldon Domain and Transmembrane Domain (NTD-RBD-FD-TM)

| | |
|---|---|
| SEQ ID NO: 99 consists of from 5′ end to 3′ end: 5′ UTR SEQ ID NO: 2, | 99 |
| mRNA ORF SEQ ID NO: 100 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 100 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCGGCAGCGCCAUCGGCGGC | |
| | UACAUCCCCGAGGCCCCUAGAGACGGCCAGGCCUACGUGCGGAAGG | |
| | ACGGCGAGUGGGUGCUGCUGAGCACCUUCCUGGGCGGAGGCAGCAU | |
| | CCUGGCCAUCUACAGCACCGUGGCCAGCAGCCUGGUGCUGCUGGUG | |
| | AGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 101 |
| acid sequence | VLHSTQDLFLPFFSNVTWFHAIHVSGTNGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsggggSAIGG | |
| | YIPEAPRDGQAYVRKDGEWVLLSTFLGggsilaiystvasslvllv | |
| | slgaisf | |
| PolyA tail | 100 nt | |

SARS-CoV-2 NTD-RBD Linked to Transmembrane Domain and Foldon Domain (NTD-RBD-TM-FD)

| | |
|---|---|
| SEQ ID NO: 102 consists of from 5′ end to 3′ end: 5′ UTR SEQ ID NO: 2, | 102 |
| mRNA ORF SEQ ID NO: 103 and 3′ UTR SEQ ID NO: 4. | |

| | | |
|---|---|---|
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 103 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUCGGCGGAGGCAGCGCCAUCGGCGGCUACAUCCCCGAGGC | |
| | CCCUAGAGACGGCCAGGCCUACGUGCGGAAGGACGGCGAGUGGGUG | |
| | CUGCUGAGCACCUUCCUGGGCAAG | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 104 |
| acid sequence | VLHSTQDLFLPFFSNVTWFHAIHVSGTNGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsgggsilaiy | |
| | stvasslvllvslgaisfgggSAIGGYIPEAPRDGQAYVRKDGEWV | |
| | LLSTFLGk | |
| PolyA tail | 100 nt | |

SARS-CoV-2 NTD-RBD Linked to Foldon Domain

| | |
|---|---|
| SEQ ID NO: 111 consists of from 5′ end to 3′ end: 5′ UTR SEQ ID NO: 2, | 111 |
| mRNA ORF SEQ ID NO: 112 and 3′ UTR SEQ ID NO: 4. | |

| | | |
|---|---|---|
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 112 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACCUGUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGGAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCGGCAGCGCCAUCGGCGGC | |
| | UACAUCCCCGAGGCCCCUAGAGACGGCCAGGCCUACGUGCGGAAGG | |
| | ACGGCGAGUGGGUGCUGCUGAGCACCUUCCUGGGC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLITRTQLPPAYTNSFTRGVYYPDKVERSS | 113 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYLYRLFRKSNLKPFERDISTEIYQAGSTPCNGVEGENCYFPL | |
| | QSYGFQPTNGVGYQPYRVVVLSFELLHAPATVCGPKsggggSAIGG | |
| | YIPEAPRDGQAYVRKDGEWVLLSTFLG | |
| PolyA tail | 100 nt | |

### Encapsulin

In some embodiments, an encapsulin domain is used as a scaffold domain. Encapsulin is a protein cage nanoparticle isolated from the thermophile Thermotoga maritima. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008; 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses 30 (Rahmanpour R. et A. FEBS J. 2013; 280: 2097-2104). The use of encapsulin for nanoparticle construction enables both the display of protein antigen on the surface of the nanoparticle, and the enclosure of cargo such as mRNA within the nanoparticle itself. Previous encapsulin nanoparticle-based vaccines have elicited strong immune responses to both surface displayed antigen and cargo protein itself (Lagoutte P. et al. Vaccine. 2018; 36(25): 3622-3628).

An mRNA provided herein, in some embodiments, encodes an S protein domain (e.g., S1, S2, RBD, and/or NTD) linked to an encapsulin domain.

### Fusion Proteins

In some embodiments, a composition of the present disclosure includes an mRNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

In some embodiments, a fusion protein comprises a receptor binding domain from a SARS-CoV-2 Spike protein.

In some embodiments, a fusion protein comprises an N-terminal domain from a SARS-CoV-2 Spike protein

In some embodiments, a fusion protein comprises a transmembrane domain. The transmembrane domain may, in some embodiments, be from a virus that is not SARS-CoV-2. For example, the transmembrane domain may be from an influenza hemagglutinin transmembrane domain, which has been demonstrated to effectively anchor proteins at the cell surface.

### Variants

In some embodiments, the compositions of the present disclosure include RNA that encodes a coronavirus antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an mRNA or an mRNA ORF that comprises a nucleotide sequence of any one of the sequences provided herein (see, e.g., Sequence Listing), or comprises a nucleotide sequence at least 80%, at least 85%, at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, particularly the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an mRNA having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally-occurring or wild-type sequence (e.g., a naturally-occurring or wild-type mRNA sequence encoding a coronavirus antigen). In some embodiments, a codon optimized sequence shares less than 85% sequence identity to a naturally-occurring or wild-type sequence (e.g., a naturally-occurring or wild-type mRNA sequence encoding a coronavirus antigen). In some embodiments, a codon optimized sequence shares less than 80% sequence identity to a naturally-occurring or wild-type sequence (e.g., a naturally-occurring or wild-type mRNA sequence encoding a coronavirus antigen). In some embodiments, a codon optimized sequence shares less than 75% sequence identity to a naturally-occurring or wild-type sequence (e.g., a naturally-occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a coronavirus antigen). In some embodiments, a codon optimized sequence shares between 65% and 75% or about 80% sequence identity to a naturally-occurring or wild-type sequence (e.g., a naturally-occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than mRNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an mRNA having an open reading frame encoding a coronavirus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an mRNA of the disclosure comprises 1-methyl-pseudouridine (m1ψ) substitutions at one or more or all uridine positions of the nucleic acid.

In some embodiments, an mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, an mRNA of the disclosure comprises pseudouridine (ψ) substitutions at one or more or all uridine positions of the nucleic acid.

In some embodiments, an mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, an mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 128), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 129) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 131 and SEQ ID NO: 2.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 130) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

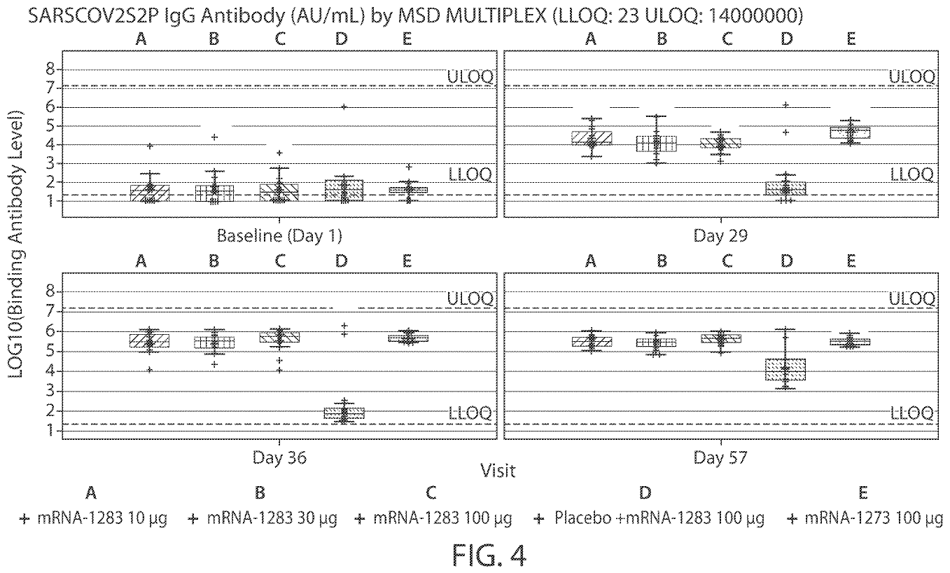

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). Addtionally, a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit p globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG. 4 (WO2015101415), and human albumin 7 (WO2015101415).

In some embodiments, a 3′ UTR of the present disclosure comprises a sequence selected from SEQ ID NO: 132 and SEQ ID NO: 4.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

In vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an mRNA, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle (LNP). Lipid nanoparticles typically comprise ionizable amino lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %.

In some embodiments, the lipid nanoparticle comprises 5-15 mol % helper lipid, optionally 10-12 mol %, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol %.

In some embodiments, the lipid nanoparticle comprises 1-5% PEG lipid, optionally 1-3 mol %, for example 1.5 to 2.5 mol %, 1-2 mol %, 2-3 mol %, 3-4 mol %, or 4-5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, an ionizable amino lipid of the disclosure comprises a compound of Formula (I):

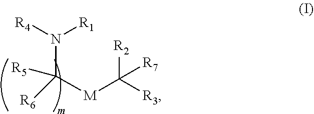

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR,—CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,—CHQR,—CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁_₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃_₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,—CHQR,—CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)n CHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃_₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂_s alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)·CHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

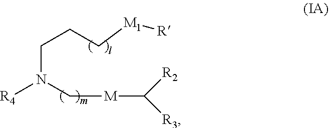

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)n Q, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

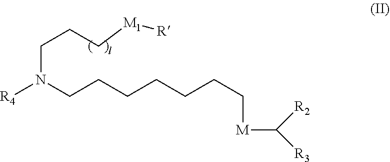

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

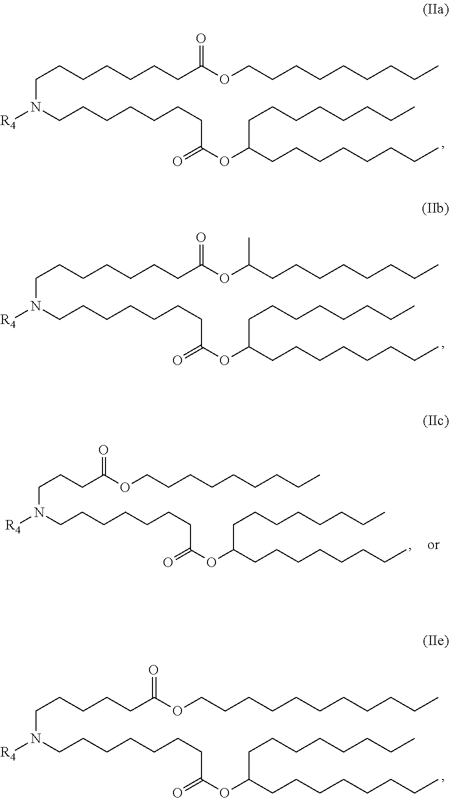

or a salt or isomer thereof, wherein R₄ is as described herein.

In some embodiments, a subset of compounds of Formula (I) includes those of Formula (IId):

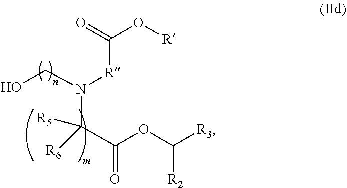

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

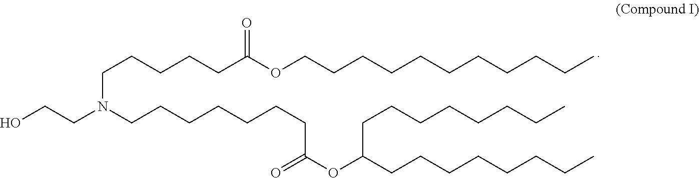 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

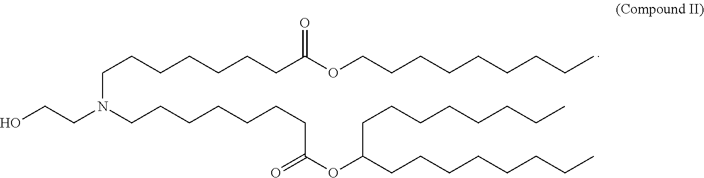 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

In some embodiments, the lipid nanoparticle comprises 1-2 mol %, 1-3 mol %, 1-4 mol %, or 1-5 mol % DMG-PEG. For example, the lipid nanoparticle may comprise 1, 1.5, 2, 2.5, 3, or 3.5 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG.

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, an LNP of the disclosure comprises an N:P ratio of from about 2:1 to about 30:1.

In some embodiments, an LNP of the disclosure comprises an N:P ratio of about 6:1.

In some embodiments, an LNP of the disclosure comprises an N:P ratio of about 3:1.

In some embodiments, an LNP of the disclosure comprises a wt/wt ratio of the ionizable amino lipid component to the RNA of from about 10:1 to about 100:1.

In some embodiments, an LNP of the disclosure comprises a wt/wt ratio of the ionizable amino lipid component to the RNA of about 20:1.

In some embodiments, an LNP of the disclosure comprises a wt/wt ratio of the ionizable amino lipid component to the RNA of about 10:1.

In some embodiments, an LNP of the disclosure has a mean diameter from about 50 nm to about 150 nm.

In some embodiments, an LNP of the disclosure has a mean diameter from about 70 nm to about 120 nm.

### Multivalent Vaccines

The compositions, as provided herein, may include RNA or multiple RNAs encoding two or more antigens of the same or different virus species. In some embodiments, composition includes an mRNA or multiple mRNAs encoding two or more coronavirus antigens. In some embodiments, the RNA may encode 1, 2, 3, 4, 5, 6, 7, 8, 9, 10, 11, 12, or more coronavirus antigens.

In some embodiments, two or more different mRNA encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The compositions, as provided herein, may include an mRNA or multiple RNAs encoding two or more antigens of the same or different viral strains. Also provided herein are combination vaccines that include RNA encoding one or more coronavirus and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus.

### Pharmaceutical Formulations

Provided herein are compositions (e.g., pharmaceutical compositions), methods, kits and reagents for prevention or treatment of coronavirus in humans and other mammals, for example. The compositions provided herein can be used as therapeutic or prophylactic agents. They may be used in medicine to prevent and/or treat a coronavirus infection.

In some embodiments, the coronavirus vaccine containing RNA as described herein can be administered to a subject (e.g., a mammalian subject, such as a human subject), and the mRNAs are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing mRNA having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a coronavirus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. In some embodiments, the subject is administered at least one additional booster dose, such that the subject is administered 1, 2, 3, 4, 5, 6, 7, 8, 9, 10, or more booster doses. The time of administration between the initial administration of the prophylactic composition and the booster (or between booster doses) may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In exemplary embodiments, the time of administration between the initial administration of the prophylactic composition and the booster may be, but is not limited to, 1 week, 2 weeks, 3 weeks, 1 month, 2 months, 3 months, 6 months or 1 year. In some embodiments, the booster dose is administered at least 28 days after a first dose of the composition is administered to the subject and within one year of the first dose.

In some embodiments, a composition may be administered intramuscularly, intranasally or intradermally, similarly to the administration of inactivated vaccines known in the art. In some embodiments, the composition is administered intramuscularly, for example, to a subject's deltoid muscle.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, a composition may comprise other components including, but not limited to, adjuvants.

In some embodiments, a composition does not include an adjuvant (they are adjuvant free).

An RNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, a composition is administered to humans, human patients or subjects. For the purposes of the present disclosure, the phrase “active ingredient” generally refers to the RNA vaccines or the polynucleotides contained therein, for example, mRNA encoding antigens.

Formulations of the vaccine compositions described herein may be prepared by any method known or hereafter developed in the art of pharmacology. In general, such preparatory methods include the step of bringing the active ingredient (e.g., mRNA) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an mRNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are compositions (e.g., RNA vaccines), methods, kits and reagents for prevention and/or treatment of coronavirus infection in humans and other mammals. Immunizing compositions can be used as therapeutic or prophylactic agents. In some embodiments, compositions are used to provide prophylactic protection from coronavirus infection. In some embodiments, compositions are used to treat a coronavirus infection. In some embodiments, embodiments, compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, a composition (e.g., RNA a vaccine) is administered to a subject (e.g., a mammalian subject, such as a human subject) in an effective amount to induce an antigen-specific immune response. The RNA encoding the coronavirus antigen is expressed and translated in vivo to produce the antigen, which then stimulates an immune response in the subject.

Prophylactic protection from a coronavirus can be achieved following administration of a composition of the present disclosure. Immunizing compositions can be administered once, twice, three times, four times or more but it is likely sufficient to administer the vaccine once (optionally followed by a single booster). It is possible, although less desirable, to administer a composition to an infected individual to achieve a therapeutic response. Dosing may need to be adjusted accordingly.

A method of eliciting an immune response in a subject against a coronavirus antigen (or multiple antigens) is provided in aspects of the present disclosure. In some embodiments, a method involves administering to the subject a composition comprising a mRNA having an open reading frame encoding a coronavirus antigen, thereby inducing in the subject an immune response specific to the coronavirus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the coronavirus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the coronavirus or an unvaccinated subject.

A method of eliciting an immune response in a subject against a coronavirus is provided in other aspects of the disclosure. The method involves administering to the subject a composition comprising an mRNA comprising an open reading frame encoding a coronavirus antigen, thereby inducing in the subject an immune response specific to the coronavirus, wherein the immune response in the subject is equivalent to an immune response in a subject vaccinated with a traditional vaccine against the coronavirus at 2 times to 100 times the dosage level relative to the composition.

In some embodiments, the immune response in the subject is equivalent to an immune response in a subject vaccinated with a traditional vaccine at twice the dosage level relative to a composition of the present disclosure. In some embodiments, the immune response in the subject is equivalent to an immune response in a subject vaccinated with a traditional vaccine at three times the dosage level relative to a composition of the present disclosure. In some embodiments, the immune response in the subject is equivalent to an immune response in a subject vaccinated with a traditional vaccine at 4 times, 5 times, 10 times, 50 times, or 100 times the dosage level relative to a composition of the present disclosure. In some embodiments, the immune response in the subject is equivalent to an immune response in a subject vaccinated with a traditional vaccine at 10 times to 1000 times the dosage level relative to a composition of the present disclosure. In some embodiments, the immune response in the subject is equivalent to an immune response in a subject vaccinated with a traditional vaccine at 100 times to 1000 times the dosage level relative to a composition of the present disclosure.

In other embodiments, the immune response is assessed by determining [protein] antibody titer in the subject. In other embodiments, the ability of serum or antibody from an immunized subject is tested for its ability to neutralize the virus. In other embodiments, the ability to promote a robust T cell response(s) is measured using art recognized techniques.

Other aspects the disclosure provide methods of eliciting an immune response in a subject against a coronavirus by administering to the subject composition comprising an mRNA having an open reading frame encoding a coronavirus antigen, thereby inducing in the subject an immune response specific to the coronavirus antigen, wherein the immune response in the subject is induced 2 days to 10 weeks earlier relative to an immune response induced in a subject vaccinated with a prophylactically effective dose of a traditional vaccine against the coronavirus. In some embodiments, the immune response in the subject is induced in a subject vaccinated with a prophylactically effective dose of a traditional vaccine at 2 times to 100 times the dosage level relative to a composition of the present disclosure.

Also provided herein are methods of eliciting an immune response in a subject against a coronavirus by administering to the subject an mRNA having an open reading frame encoding a first antigen, wherein the RNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount (e.g., therapeutic dose) of the RNA, as provided herein, may be as low as 20 μg, administered for example as a single dose or as two 10 μg doses. In some embodiments, the effective amount (e.g., therapeutic dose) is a total dose of 10 μg. In some embodiments, the effective amount (e.g., therapeutic dose) is a total dose of 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the therapeutic dose (e.g., effective amount) is at least 10 μg and less than 25 μg of the composition. In some embodiments, the therapeutic dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the compositions (e.g., RNA vaccines), wherein the RNA is formulated in an effective amount to produce an antigen specific immune response in a subject (e.g., production of antibodies specific to a coronavirus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) coronavirus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4⁺ helper and/or CD8⁺ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-coronavirus antigen antibody titer produced in a subject administered a composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, SAR-CoV-2 virus or SAR-CoV-2 viral antigen, e.g., SAR-CoV-2 spike or S protein, of domain thereof. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection.

The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding SARS-CoV-2 neutralizing antibodies, the “seroprotective” threshold titer remains unknown; but a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by a composition (e.g., RNA vaccine).

In some embodiments, an anti-coronavirus antigen antibody titer produced in a subject is increased by at least 1 log relative to a control. For example, anti-coronavirus antigen antibody titer produced in a subject may be increased by at least 1.5, at least 2, at least 2.5, or at least 3 log relative to a control. In some embodiments, the anti-coronavirus antigen antibody titer produced in the subject is increased by 1, 1.5, 2, 2.5 or 3 log relative to a control. In some embodiments, the anti-coronavirus antigen antibody titer produced in the subject is increased by 1-3 log relative to a control. For example, the anti-coronavirus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-coronavirus antigen antibody titer produced in a subject is increased at least 2 times relative to a control. For example, the anti-coronavirus antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-coronavirus antigen antibody titer produced in the subject is increased 2, 3, 4, 5, 6, 7, 8, 9, or 10 times relative to a control. In some embodiments, the anti-coronavirus antigen antibody titer produced in a subject is increased 2-10 times relative to a control. For example, the anti-coronavirus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to coronavirus. A geometric mean titer (GMT) is the average antibody titer for a group of subjects calculated by multiplying all values and taking the nth root of the number, where n is the number of subjects with available data.

A control, in some embodiments, is an anti-coronavirus antigen antibody titer produced in a subject who has not been administered a composition (e.g., RNA vaccine). In some embodiments, a control is an anti-coronavirus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of a composition (e.g., RNA vaccine) to be effective is measured in a murine model. For example, a composition may be administered to a murine model and the murine model assayed for induction of neutralizing antibody titers. Viral challenge studies may also be used to assess the efficacy of a vaccine of the present disclosure. For example, a composition may be administered to a murine model, the murine model challenged with virus, and the murine model assayed for survival and/or immune response (e.g., neutralizing antibody response, T cell response (e.g., cytokine response)).

In some embodiments, an effective amount of a composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent coronavirus infection or a related condition, while following the standard of care guideline for treating or preventing coronavirus infection or a related condition.

In some embodiments, the anti-coronavirus antigen antibody titer produced in a subject administered an effective amount of a composition is equivalent to an anti-coronavirus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$\begin{matrix}Efficacy = ( ARU - ARV ) / ARU \times 100 ; and \\ Efficacy = ( 1 - RR ) \times 100.\end{matrix}$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, efficacy of the composition (e.g., RNA vaccine) is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition of the present disclosure is sufficient to provide sterilizing immunity in the subject for at least 1 year. For example, the effective amount of a composition of the present disclosure is sufficient to provide sterilizing immunity in the subject for at least 2 years, at least 3 years, at least 4 years, or at least 5 years. In some embodiments, the effective amount of a composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition of the present disclosure is sufficient to produce detectable levels of coronavirus antigen as measured in serum of the subject at 1-72 hours post administration.

Titer. An antibody titer is a measurement of the number of antibodies within a subject, for example, antibodies that are specific to a particular antigen (e.g., an anti-coronavirus antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a composition of the present disclosure is sufficient to produce a 1,000-10,000 neutralizing antibody titer produced by neutralizing antibody against the coronavirus antigen as measured in serum of the subject at 1-72 hours post administration. In some embodiments, the effective amount is sufficient to produce a 1,000-5,000 neutralizing antibody titer produced by neutralizing antibody against the coronavirus antigen as measured in serum of the subject at 1-72 hours post administration. In some embodiments, the effective amount is sufficient to produce a 5,000-10,000 neutralizing antibody titer produced by neutralizing antibody against the coronavirus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-coronavirus antigen antibody titer produced in the subject is increased by at least 1 log relative to a control. For example, an anti-coronavirus antigen antibody titer produced in the subject may be increased by at least 2, 3, 4, 5, 6, 7, 8, 9 or 10 log relative to a control.

In some embodiments, an anti-coronavirus antigen antibody titer produced in the subject is increased at least 2 times relative to a control. For example, an anti-coronavirus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### EXAMPLES

### Example 1. Phase I Clinical Trial

### Study Design and Methodology

The study is a Phase 1, randomized, observer-blind study in healthy adult participants 18 to 55 years of age, who are in good health and meet all eligibility criteria. The study is used to assess the safety, reactogenicity, and immunogenicity of a SARS-CoV-2 mRNA vaccine (SEQ ID NO: 90; ORF SEQ ID NO: 91; encoding SEQ ID NO: 92; referred to herein as “mRNA vaccine”). All participants participate in a Screening Period, Treatment Period, and Follow up Period.

Three dose levels (10 μg, 30 μg, and 100 μg) of the mRNA vaccine (Arms 1 through 3) and 1 dose level (100 μg) of the mRNA vaccine (Arm 5) will each be evaluated in a 2-dose regimen, with the doses administered 28 days apart. One dose level (100 μg) of the mRNA vaccine will be evaluated in a single-dose regimen (Arm 4). Approximately 125 participants will be randomized in a 1:1:1:1:1 ratio to receive an investigational product (IP), with approximately 25 participants randomized to each study arm. All study arms will be enrolled in parallel. Participants receive 2 doses of IP by 0.5 mL intramuscular (IM) injection on Day 1 and/or Day 29, with the exception of participants in Arm 4 (single-dose administration of the mRNA vaccine), who will receive a placebo injection on Day 1 and IP on Day 29. Each injection has a volume of 0.5 mL and is injected intramuscularly into the deltoid muscle. The administration schedule is shown in the table below.

| | Drug | | | | |
|---|---|---|---|---|---|
| Arm No. | Administered | Initial Dose | Interval | Boost Dose | N |
| 1 | mRNA vaccine | 10 μg | 28 days | 10 μg | 25 |
| 2 | mRNA vaccine | 30 μg | 28 days | 30 μg | 25 |
| 3 | mRNA vaccine | 100 μg | 28 days | 100 μg | 25 |
| 4 | mRNA vaccine | Placebo | 28 days | 100 μg | 25 |
| 5 | mRNA vaccine | 100 μg | 28 days | 100 μg | 25 |

Nonclinical studies in Balb/c mice assessed immunogenicity by evaluating binding antibody (bAb) and neutralizing antibody (nAb) responses as well as Th1-directed CD4+ and CD8+ responses elicited by the mRNA vaccine. The mRNA vaccine was shown to be immunogenic in Balb/c mice, demonstrating a bAb response and neutralization activity. The mRNA vaccine elicited CD4+ T cells re-stimulated with S1 or S2 peptide pools exhibited a Th1 dominant response (production of interferon-γ, interleukin-2, tumor necrosis factor-α), particularly at higher immunogen doses. Spike 1 and S2 in combination represent a peptide pool covering the entire SARS-CoV-2 S protein; the peptides were split into 2 pools to increase stability. Furthermore, the mRNA vaccine induced a robust CD8+ T cell response to the S1 peptide pool. These results show a cytokine expression profile which induced CD8+ T cells and Th1-directed CD4+ T cell responses. These data support advancement of the mRNA vaccine clinically.

### Vaccine, Dosage, and Route of Administration

The SARS-CoV-2 mRNA vaccine (SEQ ID NO: 90; ORF SEQ ID NO: 91; encoding SEQ ID NO: 92) is a lipid nanoparticle (LNP) dispersion of an mRNA in LNPs composed of four (4) lipids (50 mol % ionizable lipid heptadecan-9-yl 8 ((2 hydroxyethyl)(6 oxo 6-(undecyloxy)hexyl)amino)octanoate (Compound 1); 10 mol % 1,2 distearoyl sn glycero-3 phosphocholine (DSPC); 38.5 mol % cholesterol; and 1.5 mol % 1-monomethoxypolyethyleneglycol-2,3-dimyristylglycerol with polyethylene glycol of average molecular weight 2000 (PEG2000 DMG)). The SARS-CoV-2 mRNA vaccine is provided as a sterile liquid for injection, white to off white dispersion in appearance, at a concentration of 0.5 mg/mL in 20 mM trometamol (Tris) buffer containing 87 mg/mL sucrose and 10.7 mM sodium acetate, at pH 7.5.

The diluent is 0.9% sodium chloride (normal saline) injection, United States Pharmacopeia (USP). The USP grade 0.9% NaCl or normal saline for injection is a sterile, nonpyrogenic, isotonic solution; each mL contains NaCl 9 mg. It contains no bacteriostatic agent, antimicrobial agent, preservatives, or added buffer and is supplied only in single-dose containers. The solution may contain hydrochloric acid and/or sodium hydroxide for pH adjustment (pH 5.3, range 4.5-7.0). This product should be used to dilute the vaccine to the desired concentration.

The SARS-CoV-2 mRNA vaccine is administered as an intramuscular injection into the deltoid muscle on a 2-dose vaccination schedule on Day 1 and Day 29, with at least a 28-day interval between doses. Each vaccination contains 0.5 mL of the SARS-CoV-2 mRNA vaccine diluted in 0.9% NaCl for injection, USP, to obtain 10 μg, 30 μg, or 100 μg of the mRNA vaccine in each 0.5 mL dose.

### Objectives

The primary objective is to evaluate the safety and reactogenicity of a 2-dose vaccination schedule of the mRNA vaccine (SEQ ID NO: 90; ORF SEQ ID NO: 91; encoding SEQ ID NO: 92), given 28 days apart, across 3 dosages (10 μg, 30 μg, or 100 μg); and of one high-dose level (100 μg) of the mRNA vaccine administered as a single dose in healthy adults.

The secondary objectives are to evaluate the immunogenicity of the mRNA vaccine at the three different administration schedules by assessing the titer or level of neutralizing antibody and binding antibody.

The exploratory objectives are (1) to assess SARS-CoV-2 S-specific cell-mediated immune responses; (2) to further characterize humoral immune responses; and (3) to characterize the immune response of participants infected by SARS-CoV-2 during the study.

The full study comprises 8 scheduled study site visits: Screening, Day 1, Day 8, Day 29 (Month 1), Day 36, Day 57 (Month 2), Day 209 (Month 7), and Day 394 (Month 13). To test for the presence of severe acute respiratory syndrome coronavirus 2 (SARS-CoV-2) virus, swab samples are collected on Day 1, Day 29, Day 57, Day 209, and Day 394. Participants have serum collected at Screening and Day 1, Day 29, Day 57, and Day 394 for subsequent testing for anti-nucleocapsid antibodies detected by immunoassay. All participants are followed for safety and reactogenicity and will provide pre- and post-injection blood specimens for immunogenicity through 12 months after the last dose of IP.

Immunogenicity is measured by measuring serum binding antibody level against SARS-CoV-2 with an enzyme-linked immunosorbent assay (ELISA) specific to the SARS CoV 2 S protein and receptor binding domain (RBD). In addition, serum neutralizing antibody levels against SARS-CoV-2 are measured by pseudovirus and/or live virus neutralization assays. Further, testing for serologic markers for SARS-CoV-2 infection is performed by measuring anti-nucleocapsid antibodies detected by immunoassay.

Participants have nasopharyngeal (NP) samples collected for SARS-CoV-2 testing on Days 1, 29, 57, 209, and 394. A study illness visit or a consultation is arranged within 24 hours or as soon as possible to collect an NP or nasal swab sample to ascertain the presence of SARS-CoV-2 via reverse transcriptase PCR if a participant experiences any of the following: signs or symptoms of SARS-CoV-2 infection as defined by the CDC; exposure to an individual confirmed to be infected with SARS-CoV-2; medically-attended adverse event (MAAE) suggesting a SARS-CoV-2 infection. Additionally, clinical information is collected to evaluate the severity of the clinical case.

### Example 2. Interim Analysis Results

Interim analysis was conducted on 104 randomized participants. Per-protocol immunogenicity analyses included 86 participants who received 2 doses of study vaccine and completed the Day 57 visit, were anti-NP antibody-negative and RT-PCR negative at baseline, without major protocol deviations. A safety follow-up was performed after dose 2 at a median of 127 days (>4 months). No SAEs or AESIs have been reported to date and no study pause rules have been met. Day 57 immunogenicity results indicate that all dose levels (10, 30, and 100 μg) of mRNA-1283 administered as two doses, 28 days apart, induce similar neutralizing and binding antibody titers or levels compared to mRNA-1273 for prototype SARS-CoV-2 (D614G) and similar pseudovirus neutralizing antibody titers to the B.1.351 (beta) variant. Systemic solicited adverse events, specifically for fever and chills, were observed more frequently at higher dose levels of mRNA-1283 dose compared to mRNA-1273. However, mRNA-1283 at 10 μg may be better tolerated than mRNA-1273. These results indicate that mRNA-1283 is more potent than mRNA-1273 and similar effectiveness may be achieved at a lower dose level. Results are summarized in Tables 16 and 17, below.

TABLE 16
Summary of Pseudovirus nAb ID50 titers against prototype (Per-
Protocol Set for Immunogenicity; LLOQ: 18.5, ULOQ: 45118)

| | | | | |
|---|---|---|---|---|
| 1283 | 1283 | 1283 | 1283 | 1273 |
| 10 ug | 30 ug | 100 ug | Placebo + 100 ug | 100 ug |
| (Cohort 1) | (Cohort 2) | (Cohort 3) | (Cohort 4) | (Cohort 5) |
| (N = 16) | (N = 18) | (N = 20) | (N = 14) | (N = 18) |

Day 57

| | | | | | |
|---|---|---|---|---|---|
| GMT | 1370 | 1091 | 1679 | 59 | 1190 |
| 95% CI | 888, 2112 | 645, 1846 | 1133, 2487 | 17, 206 | 826, 1713 |
| GMR | 1.15 | 0.92 | 1.41 | 0.05 | N/A |
| (1283 vs 1273) | | | | | |
| 95% CI | 0.671, 1.975 | 0.495, 1.699 | 0.838, 2.377 | 0.014, 0.179 | N/A |
| GMFR | 148 | 90 | 182 | 4 | 129 |
| 95% CI | 96, 228 | 48, 170 | 123, 269 | 2, 11 | 89, 185 |

Seroresponse

| | | | | | |
|---|---|---|---|---|---|
| n (%) | 16 (100) | 18 (100) | 20 (100) | 4 (28.6) | 18 (100) |
| 95% CI | 79, 100 | 82, 100 | 83, 100 | 8, 58 | 82, 100 |

TABLE 17
Summary of Pseudovirus nAb ID50 titers against B.1.351(Per-
Protocol Set for Immunogenicity; LLOQ: 19.5, ULOQ: 385.7)

| | | | | |
|---|---|---|---|---|
| 1283 | 1283 | 1283 | 1283 | 1273 |
| 10 ug | 30 ug | 100 ug | Placebo + 100 ug | 100 ug |
| (Cohort 1) | (Cohort 2) | (Cohort 3) | (Cohort 4) | (Cohort 5) |
| (N = 16) | (N = 18) | (N = 20) | (N = 14) | (N = 18) |

Day 57

| GMT | 138.0 | 113.0 | 144.6 | 25.2 | 107.7 |
|---|---|---|---|---|---|
| 95% CI | 83.6, 227.9 | 61.4, 208.1 | 89.6, 233.5 | 8.3, 75.9 | 78.4, 148.0 |
| GMR | 1.28 | 1.05 | 1.34 | 0.23 | N/A |
| (1283 vs 1273) | | | | | |
| 95% CI | 0.735, 2.235 | 0.537, 2.053 | 0.761, 2.372 | 0.075, 0.726 | N/A |
| GMFR | 14.15 | 8.59 | 14.84 | 1.80 | 11.05 |
| 95% CI | 8.571, 23.371 | 4.231, 17.419 | 9.192, 23.946 | 0.671, 4.807 | 8.041, 15.175 |

Seroresponse

| | | | | | |
|---|---|---|---|---|---|
| n (%) | 12 (75.0) | 11 (61.1) | 17 (85.0) | 2 (14.3) | 12 (66.7) |
| 95% CI | 48, 93 | 36, 83 | 62, 97 | 2, 43 | 41, 87 |

### Additional Sequences

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 131)
GGGAAAUAAGAGAGAAAAGAAGAGUAAGAAGAAAUAUAAGAGCCA
CC
5′ UTR:
(SEQ ID NO: 2)
GGGAAAUAAGAGAGAAAAGAAGAGUAAGAAGAAAUAUAAGACCCC
GGCGCCGCCACC
3′ UTR:
(SEQ ID NO: 132)
UGAUAAUAGGCUGGAGCCUCGGUGGCCAUGCUUCUUGCCCCUUGG
GCCUCCCCCCAGCCCCUCCUCCCCUUCCUGCACCCGUACCCCCGU
GGUCUUUGAAUAAAGUCUGAGUGGGCGGC
3′ UTR:
(SEQ ID NO: 4)
UGAUAAUAGGCUGGAGCCUCGGUGGCCUAGCUUCUUGCCCCUUGG
GCCUCCCCCCAGCCCCUCCUCCCCUUCCUGCACCCGUACCCCCGU
GGUCUUUGAAUAAAGUCUGAGUGGGCGGC

SARS-CoV-2 Wild Type Spike (S) Protein

| SEQ ID NO: 123 consists of from 5′ end to 3′ end: 5′ UTR SEQ ID NO: 2, | | 123 |
|---|---|---|
| mRNA ORF SEQ ID NO: 124, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 124 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCACCCCGGAGGGCAAGGAGCGUGGCCAGCCAG | |
| | AGCAUCAUCGCCUACACCAUGAGCCUGGGCGCCGAGAACAGCGUGG | |
| | CCUACAGCAACAACAGCAUCGCCAUCCCCACCAACUUCACCAUCAG | |
| | CGUGACCACCGAGAUUCUGCCCGUGAGCAUGACCAAGACCAGCGUG | |
| | GACUGCACCAUGUACAUCUGCGGCGACAGCACCGAGUGCAGCAACC | |
| | UGCUGCUGCAGUACGGCAGCUUCUGCACCCAGCUGAACCGGGCCCU | |
| | GACCGGCAUCGCCGUGGAGCAGGACAAGAACACCCAGGAGGUGUUC | |
| | GCCCAGGUGAAGCAGAUCUACAAGACCCCUCCCAUCAAGGACUUCG | |
| | GCGGCUUCAACUUCAGCCAGAUCCUGCCCGACCCCAGCAAGCCCAG | |
| | CAAGCGGAGCUUCAUCGAGGACCUGCUGUUCAACAAGGUGACCCUA | |
| | GCCGACGCCGGCUUCAUCAAGCAGUACGGCGACUGCCUCGGCGACA | |
| | UAGCCGCCCGGGACCUGAUCUGCGCCCAGAAGUUCAACGGCCUGAC | |
| | CGUGCUGCCUCCCCUGCUGACCGACGAGAUGAUCGCCCAGUACACC | |
| | AGCGCCCUGUUAGCCGGAACCAUCACCAGCGGCUGGACUUUCGGCG | |
| | CUGGAGCCGCUCUGCAGAUCCCCUUCGCCAUGCAGAUGGCCUACCG | |
| | GUUCAACGGCAUCGGCGUGACCCAGAACGUGCUGUACGAGAACCAG | |
| | AAGCUGAUCGCCAACCAGUUCAACAGCGCCAUCGGCAAGAUCCAGG | |
| | ACAGCCUGAGCAGCACCGCUAGCGCCCUGGGCAAGCUGCAGGACGU | |
| | GGUGAACCAGAACGCCCAGGCCCUGAACACCCUGGUGAAGCAGCUG | |
| | AGCAGCAACUUCGGCGCCAUCAGCAGCGUGCUGAACGACAUCCUGA | |
| | GCCGGCUGGACAAGGUGGAGGCCGAGGUGCAGAUCGACCGGCUGAU | |
| | CACUGGCCGGCUGCAGAGCCUGCAGACCUACGUGACCCAGCAGCUG | |
| | AUCCGGGCCGCCGAGAUUCGGGCCAGCGCCAACCUGGCCGCCACCA | |
| | AGAUGAGCGAGUGCGUGCUGGGCCAGAGCAAGCGGGUGGACUUCUG | |
| | CGGCAAGGGCUACCACCUGAUGAGCUUUCCCCAGAGCGCACCCCAC | |
| | GGAGUGGUGUUCCUGCACGUGACCUACGUGCCCGCCCAGGAGAAGA | |
| | ACUUCACCACCGCCCCAGCCAUCUGCCACGACGGCAAGGCCCACUU | |
| | UCCCCGGGAGGGCGUGUUCGUGAGCAACGGCACCCACUGGUUCGUG | |
| | ACCCAGCGGAACUUCUACGAGCCCCAGAUCAUCACCACCGACAACA | |
| | CCUUCGUGAGCGGCAACUGCGACGUGGUGAUCGGCAUCGUGAACAA | |
| | CACCGUGUACGAUCCCCUGCAGCCCGAGCUGGACAGCUUCAAGGAG | |
| | GAGCUGGACAAGUACUUCAAGAAUCACACCAGCCCCGACGUGGACC | |
| | UGGGCGACAUCAGCGGCAUCAACGCCAGCGUGGUGAACAUCCAGAA | |
| | GGAGAUCGAUCGGCUGAACGAGGUGGCCAAGAACCUGAACGAGAGC | |
| | CUGAUCGACCUGCAGGAGCUGGGCAAGUACGAGCAGUACAUCAAGU | |
| | GGCCCUGGUACAUCUGGCUGGGCUUCAUCGCCGGCCUGAUCGCCAU | |
| | CGUGAUGGUGACCAUCAUGCUGUGCUGCAUGACCAGCUGCUGCAGC | |
| | UGCCUGAAGGGCUGUUGCAGCUGCGGCAGCUGCUGCAAGUUCGACG | |
| | AGGACGACAGCGAGCCCGUGCUGAAGGGCGUGAAGCUGCACUACAC | |
| | C | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVERSS | 125 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNERVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNSPRRARSVASQ | |
| | SIIAYTMSLGAENSVAYSNNSIAIPTNFTISVTTEILPVSMTKTSV | |
| | DCTMYICGDSTECSNLLLQYGSFCTQLNRALTGIAVEQDKNTQEVE | |
| | AQVKQIYKTPPIKDFGGFNFSQILPDPSKPSKRSFIEDLLFNKVTL | |
| | ADAGFIKQYGDCLGDIAARDLICAQKENGLTVLPPLLTDEMIAQYT | |
| | SALLAGTITSGWTFGAGAALQIPFAMQMAYRENGIGVTQNVLYENQ | |
| | KLIANQFNSAIGKIQDSLSSTASALGKLQDVVNQNAQALNTLVKQL | |
| | SSNFGAISSVLNDILSRLDKVEAEVQIDRLITGRLQSLQTYVTQQL | |
| | IRAAEIRASANLAATKMSECVLGQSKRVDFCGKGYHLMSFPQSAPH | |
| | GVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKE | |
| | ELDKYFKNHTSPDVDLGDISGINASVVNIQKEIDRLNEVAKNLNES | |
| | LIDLQELGKYEQYIKWPWYIWLGFIAGLIAIVMVTIMLCCMTSCCS | |
| | CLKGCCSCGSCCKFDEDDSEPVLKGVKLHYT | |
| PolyA tail | 100 nt | |

It should also be understood that any one of the open reading frames and/or corresponding amino acid sequences described herein may include or exclude a signal sequence.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

What is claimed is:

1. A method comprising administering to a human subject a therapeutic dose of a composition comprising a messenger ribonucleic acid (mRNA) comprising an open reading frame (ORF) that encodes a fusion protein comprising at least two domains of a SARS-CoV-2 Spike (S) protein, and less than the full length spike protein, wherein the mRNA is in a lipid nanoparticle.

2. The method of claim 1, wherein the therapeutic dose is 30 μg of the composition.

3. The method of claim 1, wherein the therapeutic dose is a 10 μg dose of the composition.

4. The method of claim 1, wherein the therapeutic dose is 50 μg of the composition.

5. The method of claim 1, wherein the therapeutic dose is 100 μg of the composition.

6. The method of claim 1, wherein the therapeutic dose is 2.5 μg of the composition.

7. The method of claim 1, wherein the therapeutic dose is at least 10 μg and less than 25 μg of the composition.

8. The method of claim 1, wherein the therapeutic dose is at least 5 μg and less than 30 μg of the composition.

9. The method of claim 1, wherein the therapeutic dose is at least 5 μg and less than 25 μg of the composition.

10. The method of any one of claims 3-9, wherein the method comprises administering to the subject at least two doses of the composition.

11. The method of claim 10, wherein a second dose of the composition is administered to the subject at least 28 days after a first dose of the composition is administered to the subject and within one year of the first dose.

12. The method of any one of the preceding claims, wherein the composition further comprises Tris buffer.

13. The method of claim 12, wherein the composition with Tris buffer further comprises sucrose and sodium acetate.

14. The method of claim 13, wherein the composition comprises 10 mM-30 mM Tris buffer comprising 75 mg/mL-95 mg/mL sucrose, and 5 mM-15 mM sodium acetate, optionally wherein the composition has a pH of 6-8.

15. The method of claim 14, wherein the composition comprises about 20 mM Tris buffer comprising 87 mg/mL sucrose, and 10.7 mM sodium acetate, optionally wherein the composition has a pH of 7.5.

16. The method of any one of claims 1-15, wherein the composition comprises about 0.5 mg/mL of the mRNA.

17. The method of any one of the preceding claims, wherein the composition is administered intramuscularly, optionally into a deltoid muscle of the subject's arm.

18. The method of any one of the preceding claims, wherein the fusion protein comprises an amino acid sequence having at least 90%, at least 95%, or at least 98% identity to the sequence of SEQ ID NO: 92.

19. The method of claim 18, wherein the fusion protein comprises the amino acid sequence of SEQ ID NO: 92.

20. The method of any one of the preceding claims, wherein the ORF comprises a nucleotide sequence having at least 90%, at least 95%, or at least 98% identity to the sequence of SEQ ID NO: 91.

21. The method of claim 20, wherein the ORF comprises the nucleotide sequence of SEQ ID NO: 91.

22. The method of any one of the preceding claims, wherein the mRNA comprises a nucleotide sequence having at least 90%, at least 95%, or at least 98% identity to the sequence of SEQ ID NO: 90.

23. The method of claim 22, wherein the mRNA comprises the nucleotide sequence of SEQ ID NO: 90.

24. The method of any one of the preceding claims, wherein the lipid nanoparticle comprises:
ionizable amino lipid; neutral lipid; sterol; and PEG-modified lipid.

25. The method of claim 24, wherein the lipid nanoparticle comprises: 40-55 mol % ionizable amino lipid; 5-15 mol % neutral lipid; 35-45 mol % sterol; and 1-5 mol % PEG-modified lipid.

26. The method of claim 25, wherein the lipid nanoparticle comprises:
47 mol % ionizable amino lipid; 11.5 mol % neutral lipid; 38.5 mol % sterol; and 3.0 mol % PEG-modified lipid;
48 mol % ionizable amino lipid; 11 mol % neutral lipid; 38.5 mol % sterol; and 2.5 mol % PEG-modified lipid;
49 mol % ionizable amino lipid; 10.5 mol % neutral lipid; 38.5 mol % sterol; and 2.0 mol % PEG-modified lipid;
50 mol % ionizable amino lipid; 10 mol % neutral lipid; 38.5 mol % sterol; and 1.5 mol % PEG-modified lipid; or
51 mol % ionizable amino lipid; 9.5 mol % neutral lipid; 38.5 mol % sterol; and 1.0 mol % PEG-modified lipid.

27. The method of any one of claims 24-26, wherein the ionizable amino lipid is heptadecan-9-yl 8 ((2 hydroxyethyl)(6 oxo 6-(undecyloxy)hexyl)amino)octanoate (Compound 1).

28. The method of any one of claims 24-26, wherein the neutral lipid is 1,2 distearoyl sn glycero-3 phosphocholine (DSPC).

29. The method of any one of claims 24-26, wherein the sterol is cholesterol.

30. The method of any one of claims 24-26, wherein the PEG-modified lipid is 1-monomethoxypolyethyleneglycol-2,3-dimyristylglycerol with polyethylene glycol of average molecular weight 2000 (PEG2000 DMG).

31. The method of any one of the preceding claims, wherein the age of the subject is 18 to 54 years or 55 years or older.

32. The method of any one of the preceding claims, wherein the subject is immunocompromised.

33. The method of any one of the preceding claims, wherein the subject has a chronic pulmonary disease, such as chronic obstructive pulmonary disease (COPD) or asthma.

34. The method of any one of the preceding claims, wherein the subject has an underlying comorbid condition, optionally selected from obesity, heart disease, diabetes, and lung disease.

35. A composition comprising a dose of an mRNA encoding a domain of a SARS-CoV-2 Spike protein in a lipid nanoparticle, wherein the dose is at least 5 μg and less than 20 μg.

36. The composition of claim 35, wherein the dose is 10 μg.

37. A composition comprising a 30 μg dose of an mRNA encoding a domain of a SARS-CoV-2 Spike protein in a lipid nanoparticle.

38. The composition of any one of claims 35-37, wherein the domain comprises an amino (N)-terminal domain of a SARS-CoV-2 Spike protein.

39. The composition of any one of claims 35-37, wherein the domain comprises a receptor binding domain of a SARS-CoV-2 Spike protein.

40. A method comprising administering to a human subject a therapeutic dose of the composition of any one of claims 35-39 in an effective amount to produce an immune response against the domain of the SARS-CoV-2 Spike protein.

41. The method of any one of claims 1-34, wherein the geometric mean titer (GMT) of neutralizing antibody titers induced against SARS-CoV-2 (D614G) in the subject at least 45 days post administration of two doses, optionally 45 to 100 days post-administration of two doses, is at least 1,000.

42. The method of any one of claims 1-34, wherein the GMT of neutralizing antibody titers induced against SARS-CoV-2 (B.1.351) in the subject at least 45 days post administration of two doses, optionally 45 to 100 days post-administration of two doses, is at least 110.

43. The method of any one of claims 1-34, wherein the geometric mean ratio (GMR) of neutralizing antibody titers induced against SARS-CoV-2 (B.1.351) in the subject at least 45 days post administration of two doses, optionally 45 to 100 days post-administration of two doses, relative to a neutralizing antibody titer induced against SARS-CoV-2 (B.1.351) in a second subject administered mRNA encoding a SARS-CoV-2 spike protein comprising a double proline stabilizing mutation is at least 1.05.

44. The method of any one of claims 1-34, wherein the geometric mean fold rise (GMFR) of neutralizing antibody titers induced against SARS-CoV-2 (D614G) in the subject at least 45 days post administration of two doses, optionally 45 to 100 days post-administration of two doses, relative to a neutralizing antibody titer induced against SARS-CoV-2 (D614G) in the subject prior to administration of the composition is at least 90.

45. The method of any one of claims 1-34, wherein the GMFR of neutralizing antibody titers induced against SARS-CoV-2 (B.1.351) in the subject at least 45 days post administration of two doses, optionally 45 to 100 days post-administration of two doses, relative to a neutralizing antibody titer induced against SARS-CoV-2 (B.1.351) in the subject prior to administration of the composition is at least 8.
